SEQUENCE LISTING (2 sequences)

SEQ ID NO: 1 - DNA, 22 bp, Artificial Sequence
  primer
cggggatcaattcgagctcgcc

SEQ ID NO: 2 - DNA, 23 bp, Artificial Sequence
  Primer
cactgacgcagcgccagcatctt

(19) United States
(12) Patent Application Publication
(10) Pub. No.: US 2023/0263787 A1
(43) Pub. Date: Aug. 24, 2023

(54) ENHANCED MITOCHONDRIAL RESPONSE WITH THIOZOLIDINEDIONE, PIOGLITAZONE OR ROSIGLITAZONE

(71) Applicant: THE UNIVERSITY COURT OF THE UNIVERSITY OF EDINBURGH, Edinburgh (GB)

(72) Inventors: Simon LICHT-MAYER, Edinburgh, Lothian (GB); Don MAHAD, Edinburgh, Lothian (GB); Graham CAMPBELL, Edinburgh, Lothian (GB); Siddharthan CHANDRAN, Edinburgh, Lothian (GB)

(73) Assignee: THE UNIVERSITY COURT OF THE UNIVERSITY OF EDINBURGH, Edinburgh (GB)

(21) Appl. No.: 18/010,440

(22) PCT Filed: Jun. 14, 2021
(86) PCT No.: PCT/GB2021/051478

§ 371 Date: Dec. 14, 2022

(30) Foreign Application Priority Data
Jun. 15, 2020 (GB) 2009098.1

(51) Int. Cl. A61K 31/4439; A61K 31/155; A61K 31/40; A61K 31/385; A61P 25/28; G01N 33/50

(52) U.S. Cl. CPC A61K 31/4439; A61K 31/40; A61K 31/155; A61K 31/385; A61P 25/28; G01N 33/5058; G01N 33/5079; G01N 2333/70567

(57) ABSTRACT

There is provided a method of targeting mitochondrial biogenesis and mitochondrial transport from the cell body to axon to protect acutely demyelinated axons from degeneration. The method may comprise increasing mobilization of mitochondria from the neuronal cell body to the demyelinated axon to treat demyelinating disorders and provide a novel neuroprotective strategy for vulnerable acutely demyelinated axons. The method may comprise mitochondrial biogenesis in the demyelinated axon to treat demyelinating disorders and provide a novel neuroprotective strategy for vulnerable acutely demyelinated axons. The method comprises a variety of compounds and strategies for increasing mitochondrial transport and biogenesis to the demyelinated axon in both the peripheral and central nervous systems.

### FIELD OF THE INVENTION

The inventors have considered neuronal and axonal biology in relation to homeostatic axonal response of mitochondria to demyelination. Based on the axonal response of mitochondria to demyelination (ARMD) there is provided compositions and methods for neuroprotective treatments in demyelinating conditions and methods of determining the same.

### BACKGROUND

Studies performed in multiple sclerosis (MS) patients have shown that the demyelinated regions contain transected axons. In this most common inflammatory demyelinating disorder of the CNS, axon transection is most prominent in acutely demyelinated regions and evident to a lesser extent in those axons that survived the acute myelin attack and had become chronically demyelinated. Animal models of the early stage of MS (relapsing MS), experimental autoimmune encephalomyelitis, show inflammation mediated damage to myelin, mitochondria and axons, irrespective of the myelin status. Furthermore, adaptive immunity can directly transect axons, through cytotoxic T cells, presumably without involving mitochondria. These studies reflect the multiple mechanisms of axon injury in MS and highlight the major role played by inflammation at the early stage of the disease. Whilst the therapeutic goal is to to modulate inflammation, in particular the adaptive immune response, and prevent the formation of new demyelinating lesions in MS has largely been achieved, such disease modifying therapy (DMT) in relapsing MS neither prevents the transition to progressive MS (late stage) nor curtails the worsening of progressive MS. Despite the DMT, acute demyelination continues at the slowly expanding edges of established or chronic MS lesions, where acutely demyelinated axons are transected.

Demyelination contributes to the loss of axons and the progression of neurological disability in demyelinating disorders, including MS, where axons are transected during inflammatory-mediated early demyelination of white matter and later axons degenerate due to chronic demyelination.

The absence of effective treatments for progressive MS is an urgent unmet need. Whilst immune-modulatory treatments for the inflammatory demyelinating early phase of MS have had some success, the consequence of demyelination on neuronal and axonal biology remains poorly understood.

### SUMMARY OF THE INVENTION

Notably, there are no neuroprotective strategies to preserve the acutely demyelinated axon, highlighting the urgent unmet clinical need. One underlying contributor to the axonal degeneration is an imbalance between the increased energy demands of nerve conduction and the generation of ATP in the demyelinated axon.

In demyelinating disorders, such as MS, HIV neuropathy and diabetic peripheral neuropathy, perturbation of mitochondrial respiratory chain in neurons constitutes an additional contributor to the energy failure state of demyelinated axons. For example, in progressive MS, perturbation of the mitochondrial respiratory chain, including complex IV deficiency, is a robust finding in cortical neurons. Mitochondrial respiratory chain complex IV or cytochrome c oxidase (COX), the terminal complex of the electron transport chain, can be deficient in multiple neuronal subtypes. Complex IV deficiency in neurons impairs anterograde mitochondrial transport, depletes axonal mitochondria and causes degeneration of myelinated axons [21]. As the loss of complex IV deficient neurons is a late phenomenon, demyelination of these neurons will further compromise the energy failure state.

As discussed, an underlying contributor to the axonal degeneration is an imbalance between the increased energy demands of nerve conduction and the generation of ATP in the demyelinated axon. To help compensate for this imbalance, axonal mitochondrial content is consistently increased following demyelination.

Myelination is known to conserve neuronal energy by clustering voltage-gated Na channels to the nodes of Ranvier, enabling saltatory conduction of action potentials. This energy efficiency decreases the reliance on mitochondria, resulting to changes in axonal mitochondrial distribution, content and activity. Thus, the influence of myelination on axonal energy demand is akin to a brake on mitochondrial dynamics and content in the axon. In contrast, the demyelinated axon is bioenergetically challenged, given the increased reliance on the Na⁺K⁺-ATPase to maintain the resting membrane potential and axonal integrity due to the redistribution of ion channels. Consequently, mitochondria gather again in abundance in the demyelinated axons, and interrupting this exacerbates axonal degeneration.

The present inventors have shown that the mitochondrial homeostatic response to demyelination alone is insufficient to protect the axon from degeneration. Suitably, targeting mitochondrial biogenesis and mitochondrial transport from the cell body to axon, protects acutely demyelinated axons from degeneration. Crucially, enhancing mitochondrial dynamics can protect the vulnerable, acute demyelinated axons, irrespective of the presence or absence of aberrations in mitochondrial bioenergetic function. Consequently, increased mobilisation of mitochondria from the neuronal cell body to the axon is a novel neuroprotective strategy for the vulnerable, acutely demyelinated axon. The inventors propose that promoting ARMD is a crucial preceding step for implementing potential regenerative strategies for demyelinating disorders. Accordingly, there is provided a neuroprotective strategy, targeting both the early and later phases of clinical demyelinating diseases, that addresses the key substrate of neurological disability - the vulnerable axon. In addition, the inventors have discovered the importance of axonal mitochondrial respiratory chain complex IV (COX) activity in demyelinated axons. Accordingly, the invention also includes the regulation and preservation of axonal mitochondrial respiratory chain complex IV in demyelinated axons.

According to a first aspect of the present invention there is provided a method of increasing mobilization of mitochondria from the neuronal cell body to the demyelinated axon to treat demyelinating disorders and provide a novel neuroprotective strategy for vulnerable acutely demyelinated axons. It is considered promoting ARMD will, alone or in combination, provide regenerative strategies in demyelinating disorders.

Suitably the mobilisation may be provided by increasing the synthesis or production of new mitochondria (mitochondrial biogenesis) in neurons and / or over expression of proteins that are involved with the forward movement or anterograde transport of mitochondria such as kinesin family of proteins. Suitably this provides a method to protect demyelinated axons and thus be suitable to treat demyelinating disorders. Suitably the mobilisation may be provided by over expression of peroxisome proliferator-activated receptor gamma (PPAR-y) coactivator 1-alpha (PGC1a) or a down stream factor thereof, which increases the mobilisation of mitochondria in neurons. Suitably a down stream factor may be selected from a list comprising transcription factor A mitochondria (TFAM) and PPARS to increase the mobilisation of mitochondria in neurons. Suitably mobilisation may be induced by PGC1a overexpression or provision of a thiazolidinedione which increase the mobilisation of mitochondria in neurons, for example pioglitazone or rosiglitazone to a subject.

Suitably a neuroprotective strategy is provided to demyelinating diseases which is distinguished from classical treatments of neurodegenerative disorders and primary mitochondrial diseases.

Suitably the neuroprotective strategy is particularly advantageous to treat demyelinating disorders. Suitably, the demyelinating disorder may be any disorder in which axonal demyelination occurs. Suitably, the demyelinating disorder may be a disorder that causes demyelination of neurons in the central nervous system. Suitably, the demyelinating disorder may be a disorder that causes demyelination of neurons in the peripheral nervous system. Suitably, the demyelinating disorder may be a disorder that causes demyelination of neurons in both the central nervous system and peripheral nervous system. Suitably, the demyelinating disorder may be any disorder such as MS, HIV and diabetic neuropathy, chronic inflammatory demyelinating polyradiculoneuropathy (ClDP), autoimmune encephalitis, acute disseminated encephalomyelitis, transverse myelitis, Guillan-Barre Syndrome, Neuromyelitis Optica, Charcot-Marie-Tooth Disease, HTLV-l Associated Myelopathy, Balo’s disease or Schilder’s disease.

Suitably the neuroprotective strategy is particularly advantageous to treat experimentally induced demyelination or an animal model which is genetically predisposed to axonal demyelination. Suitably, the demyelination may be caused by experimental autoimmune encephalitis. Suitably, the demyelination may be caused by a T-cell receptor experimental autoimmune encephalitis transgenic mouse model. Suitably, the demyelination may be caused by Theiler’s murine encephalomyelitis virus. Suitably, the demyelination may be induced by lysolecithin. Suitably, the demyelination may be induced by lipopolysaccharide. Suitably, the demyelination may be induced by cuprizone.

Targeting mitochondrial biogenesis and mitochondrial dynamics to boost the energy producing capacity of neurons is advantageously realised by the inventors to counter demyelination which rapidly leads to an energy deficient state in axons and leads to long term damage in demyelinating disorders.

Suitably mobilisation may be provided by pharmacological application of thiazolidinediones which increase the mobilisation of mitochondria in neurons, for example pioglitazone or rosiglitazone and / or the use of small molecules and drugs that increase the anterograde transport of mitochondria in neurons can also be used, following a phenotypic screening assay of compounds and drugs.

It is considered that treatment as discussed herein would be provided as an early initiation step and until both the damage to myelin is halted and the repair of myelin or remyelination is completed. It is considered that long term treatment may be required as demyelination is ongoing in some conditions such as MS.

It is considered that the proposed neuroprotective strategy of enhancing ARMD to be effective, will advantageously be combined with effective immunomodulatory therapies and with those that restore the myelin sheath to axons (remyelination therapy) [18]. The mechanisms of axon degeneration, such as by cytotoxic T cells mediated axonal transection and by free radical mediated damage to mitochondria in both myelinated and demyelinated axons require the inflammatory response in MS to be effectively controlled [23, 30, 49, 65]. The energy imbalance in chronically demyelinated axons can be partially restored by remyelination, which decreases the axonal mitochondrial content to a level that approaches that found in myelinated axons [81]. Remyelination addresses the long term protection of axons that have survived the acute destruction of their myelin sheath (chronically demyelinated axons). In contrast, the present neuroprotection strategy allows more axons to be saved during acute demyelination so that remyelination may restore the metabolic neuronal-glial cross talk in the long term [1, 18, 20]. Thus, the present neuroprotective model serves to bridge the crucial gap between immune therapy and regenerative therapy.

Suitably, the proposed neuroprotective strategy of enhancing ARMD may be used to combat demyelination in central nervous system neurons. Suitably, the proposed neuroprotective strategy of enhancing ARMD may be used to combat demyelination in peripheral nervous system neurons. Suitably, the proposed neuroprotective strategy of enhancing ARMD may be used to combat demyelination in central and peripheral nervous system neurons.

According to a second aspect of the present invention there is provided a method of increasing mobilization of mitochondria from the neuronal cell body to the demyelinated axon to provide a novel neuroprotective strategy for vulnerable acutely demyelinated axons in combination with a method of remyelination of the axon.

Suitably any methods known in the art in relation to remyelination of the axon can be used in combination.

Suitably the present invention provides a modulator of the PGC1a/PPAR-y pathway for use in combination with a remyelination causing agent. Suitably a modulator of the PGC1a/PPAR-y pathway may be selected from thiazolidinediones which increase the mobilisation of mitochondria in neurons, for example Pioglitazone and Rosiglitazone and / or the use of small molecules and drugs that increase the anterograde transport of mitochondria in neurons. Suitably remyelination agents may be selected from Metformin, Clemestine and Lipoic acid. Suitably any agent that enhances ARMD or mitochondrial mobility can be used in combination with remyelainting agents as known in the art.

Suitably, methods of controlling inflammatory response in MS or other demyelinating diseases can be utilised in combination with enhanced ARMD as discussed herein. This may also provide a separate aspect of the invention.

Suitably methods of increasing the supply of metabolic substrate to the axon, that is necessary for mitochondrial respiratory chain function in acutely and/or chronically demyelinated axons can be utilised in combination with enhanced ARMD as discussed herein. This may also provide a separate aspect of the invention.

According to a third aspect of the invention there is provided Pioglitazone or Rosiglitazone for use in the treatment of MS, HIV neuropathy or diabetic neuropathy. In particular for use in the treatment of MS, HIV neuropathy or diabetic neuropathy in those subjects in which nerve protection is required or advantageous.

According to a fourth aspect of the present invention there is provided Pioglitazone or Rosiglitazone in combination with a remyelination agent for use in the treatment of MS, HIV neuropathy or diabetic neuropathy. In particular for use in the treatment of MS, HIV neuropathy or diabetic neuropathy in those subjects in which nerve protection is required or advantageous. Suitably, remyelination agents may be selected from Metformin, Clemestine and Lipoic acid.

Suitably in the aspects of the invention there is provided the provision of metabolic substrate and / or anti-inflammatory agent to the demyelinated axons to enhance the ARMD strategy. Metabolic substrate and / or anti-inflammatory agent may be provided with remyelination therapy.

According to a fifth aspect of the present invention, there is provided an assay method to determine modulators of the PGC1a/PPAR-y pathway comprising the steps
Providing an neuronal cell,
Applying at least a first test agent to the neuronal cell,
Determining whether the at least first test agent promotes increased mobilization of mitochondria from the neuronal cell body to the demyelinated axon,
Wherein when there is increased mobilization of mitochondria from the neuronal cell body to the demyelinated axon it is indicative that the first test agent is a modulator of the PGC1a/PPAR-y pathway.

Suitably other phenotypic screening assays which show increased mobilization of mitochondria from the neuronal cell body to the demyelinated axon could be utilised.

According to a sixth aspect of the present invention, there is provided a method of preserving or increasing the activity of axonal mitochondrial respiratory chain complex IV. Complex IV is essential for the optimal functioning of the electron transport chain and, thus, mitochondrial respiration. Complex IV is deficient within a subset of neurons in a number of demyelinating disorders such as MS, HIV and diabetes, due to decreased expression of complex IV proteins (because of mitochondrial DNA mutations). Increasing mitochondrial biogenesis in complex IV deficient neurons in complex IV knockout mice (inducible knockout), where complex IV expression is decreased, indicated a neuroprotective affect upon demyelination. In addition to this complex IV deficiency due to decreased expression of complex IV proteins, inflammatory response to demyelination frequently generate reactive oxygen species which damage mitochondria and remaining complex IV proteins via post-translational modifications. This is also evident in human diseases such as MS, and is the key driver of complex IV deficiency in experimental animal models with inflammatory demyelination. The inventors have determined that an increase in ARMD in demyelinated axons was not always accompanied with an increase in axonal mitochondrial respiratory chain complex IV (COX) activity. Furthermore, the inventors have discovered an inverse correlation between complex IV activity in demyelinated axons and axonal transection in experimental disease models. The results indicate inflammation induced post-translational modification of axonal mitochondrial proteins plays a crucial role in axonal degeneration witnessed in inflammatory conditions.

Suitably, the method may be used in the treatment of demyelinating disorders and to provide a novel neuroprotective strategy for vulnerable acutely demyelinated axons. Suitably, the method may be used in conjunction with any method to promote ARMD. Suitably, the method could be used in combination with the first to fourth aspects of the invention.

Suitably, the method could be used to preserve or enhance the activity of mitochondria which have been transported to the axon as a result of the increase in ARMD. Suitably, the method could be used to treat axonal demyelination in neurons which show a deficit in COX activity. Suitably, the method could be used to treat axonal demyelination in neurons which show a deficit in COX expression. Suitably, the method of preserving or increasing COX activity could be used in the treatment of conditions where complex IV activity is deficient, such as MS, HIV, Diabetes and rare cases of primary or inherited mitochondrial disease. Suitably, the method of preserving or increasing COX activity may be achieved by limiting damage to COX. Suitably, limiting damage to COX may be achieved through immunomodulatory therapy.

Suitably, the method of preserving or up-regulating COX activity may include the prevention of post-translational modification of COX proteins, by targeting the inflammatory response. Suitably, the method of preserving or up-regulating COX activity may include increasing mitochondrial biogenesis. Suitably, the method of up-regulating COX activity may include up-regulating the expression of the gene(s) responsible for encoding COX protein. Suitably the method may include treating a subject or neuron with Pioglitazone or Rosiglitazone.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the present invention will now be described, by way of example only, with reference to the accompanying drawings in which:

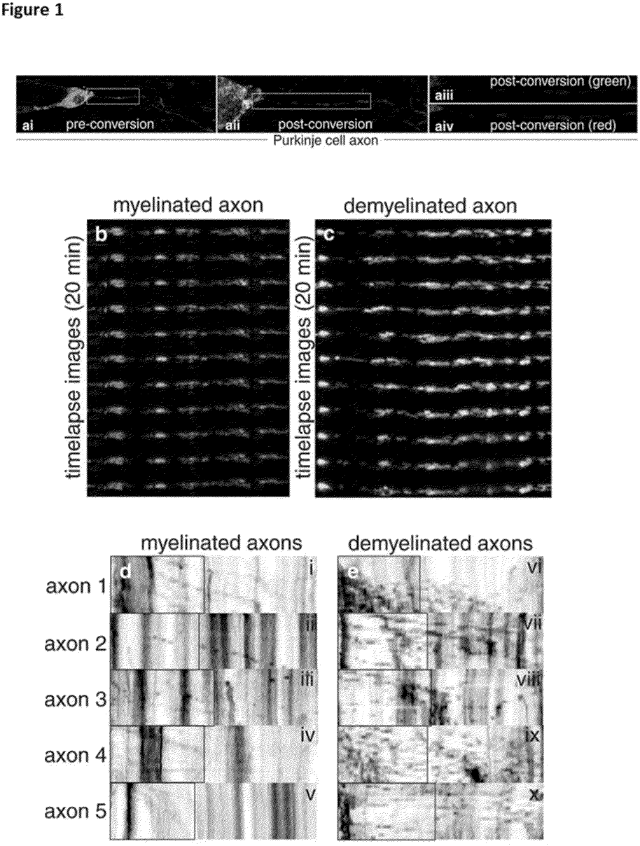

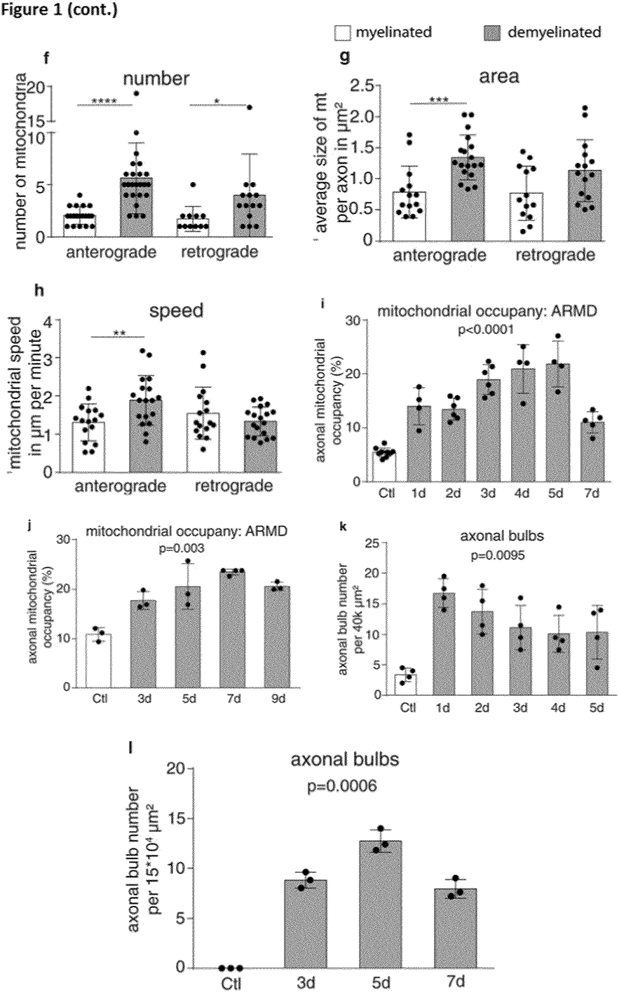

FIG. 1 illustrates demyelination mobilises mitochondria from the neuronal cell body to the axon and gradually increases in the axonal mitochondrial content.

a: Mitochondria within Purkinje cells are labelled in live cerebellar slice cultures, using lentivirus-mitochondria-targeted mEOS2 (ai, green). Photoconversion of mitochondria within the proximal axon segment adjacent to the Purkinje cell body [green to red conversion in the region of interest (ROI) shown in aii-aiv], enables the tracking of mitochondria from the cell body to the axon (anterograde, left to right).

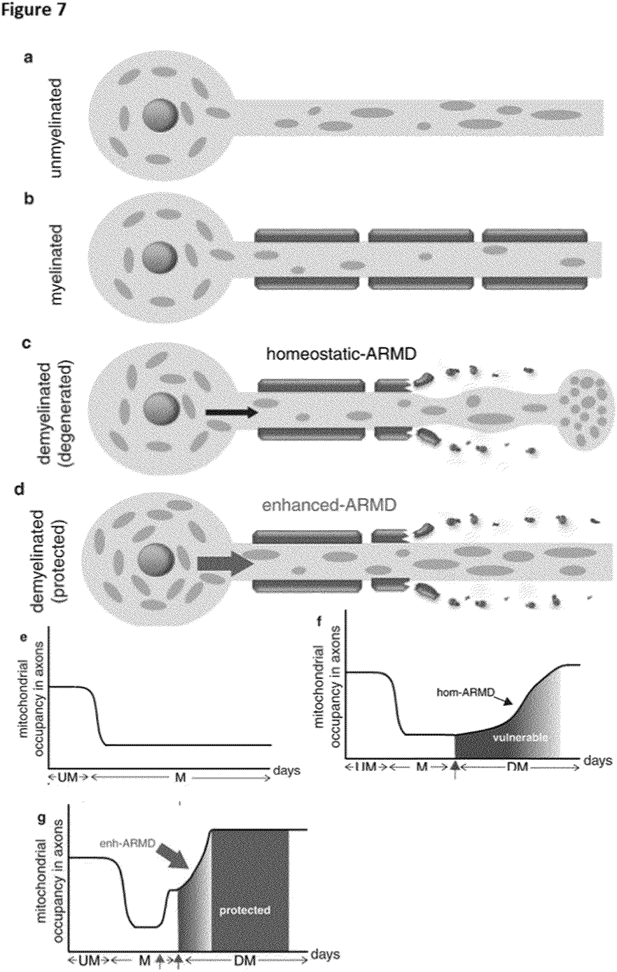

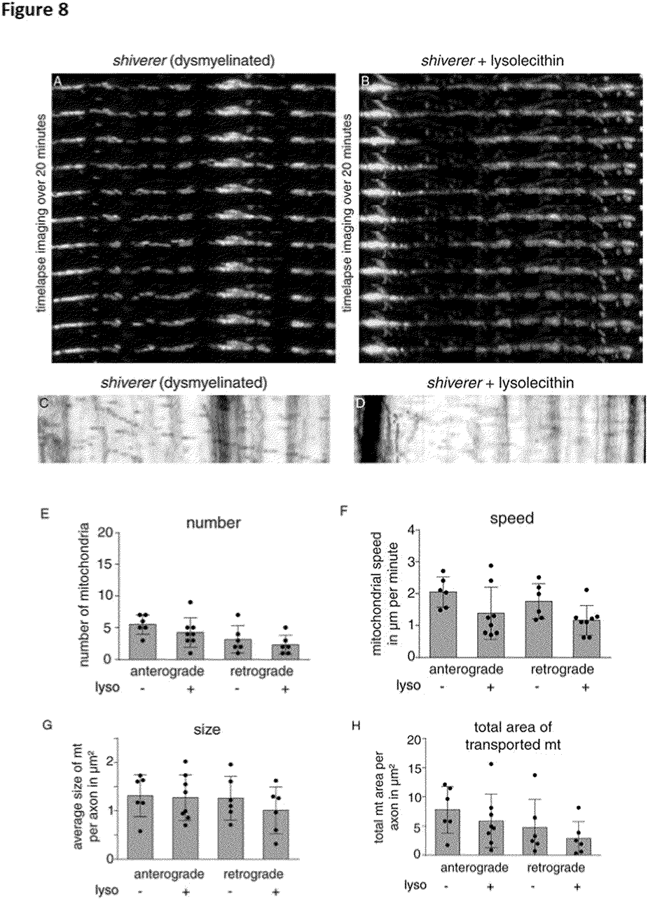

b-c: Following photoconversion, time lapse imaging of the ROI over 20 minutes shows newly transported mitochondria (green) amongst the photoconverted mitochondria (red) in a myelinated axon (b) and a demyelinated axon (c). SCoRe was used to determine the myelination status of the mEOS2 labeled axons (FIG. 8).

The majority of the photoconverted mitochondria remained stationary (b-c, red), while the newly transported mitochondria to the axon (b-c, green) continued to move (see videos for confirmation, online resource) and sometimes co-localised with the stationary mitochondria (presumably fused).

d-e: Kymographs of the green fluorescence channel show an abundance of mitochondria moving from the cell body to the ROI (left to right, anterograde transport) in demyelinated axons (e) compared with myelinated axons (d). See videos 1-5, online resource, for myelinated axons and videos 6-10, online resource, for demyelinated axons.

f-h: Quantitation of newly transported mitochondria (green) from the cell body to the ROI shows a significant increase in the number (f), area (g) and speed (h) of motile mitochondria in the demyelinated axons compared with myelinated axons. The number of retrograde moving mitochondria from the distal demyelinated axon segment to the ROI is also significantly increased upon demyelination (f). Data presented as dot-plot with mean (bar) ± standard deviation (whiskers). *p<0.05, **p<0.01, ***p<0.001, ****P<0.0001 using Mann-Whitney-U test.

i-j: Mitochondrial content of acutely demyelinated and non-transected axons gradually increases and peaks at 5 days in cerebellar slice cultures, following exposure to lysolecithin for 17 hours (i). The same peak is observed at 7 days, in vivo, following focal lysolecithin injection to the spinal cord (dorsal columns) of wild type mice (j). Each data point indicates the mean value of 20 axons from slice preparations or each animal. Statistical significance was determined using Kruskal-Wallis test.

k-l: Axonal injury following demyelination, judged by axon bulbs (transected axons), peaks 2-4 days earlier than the mitochondrial content in demyelinated axons in both cerebellar slices (k) and in vivo (I). Statistical significance was determined using Kruskal-Wallis test. ARMD: axonal mitochondrial response to demyelination.

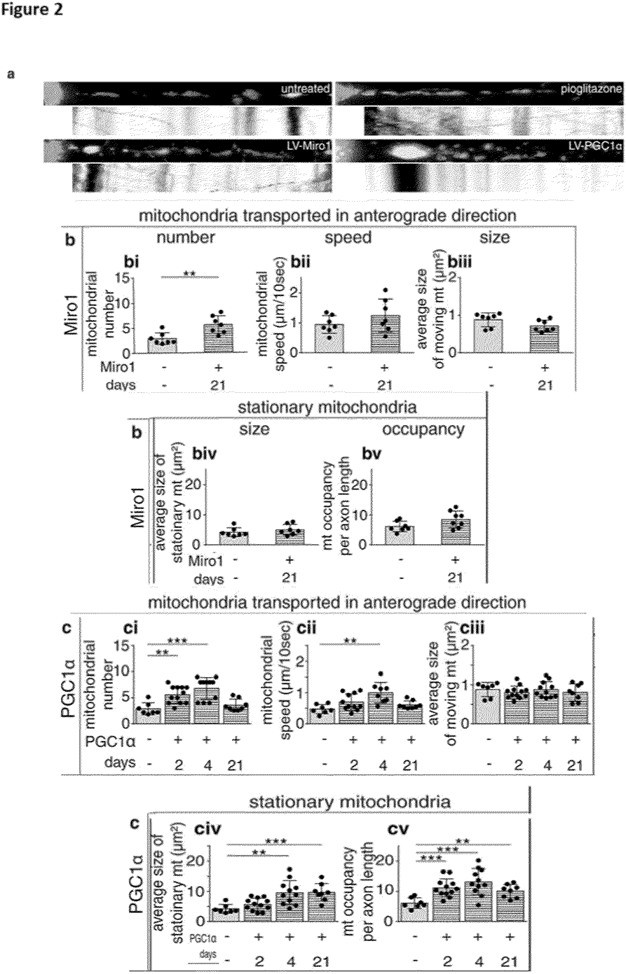

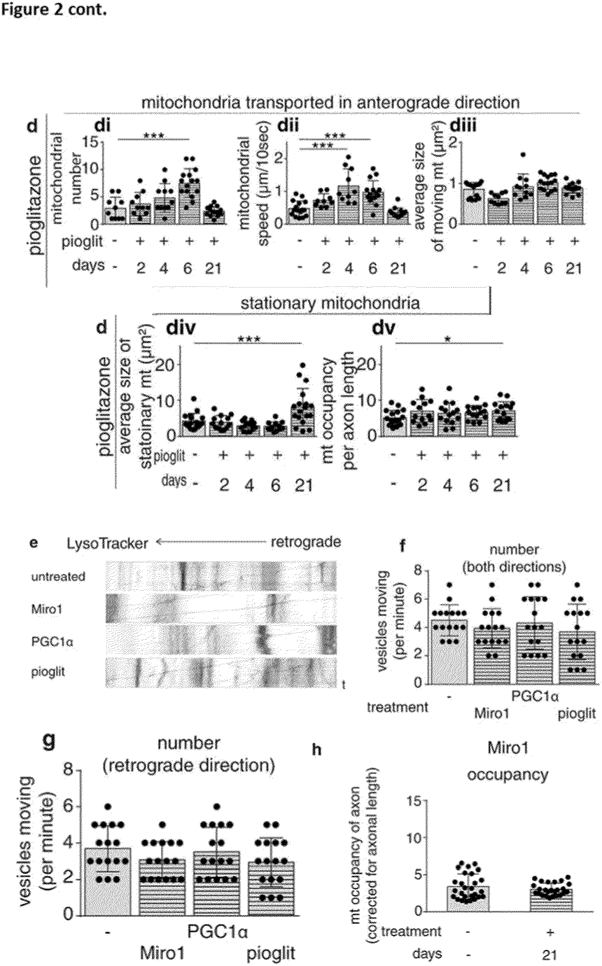

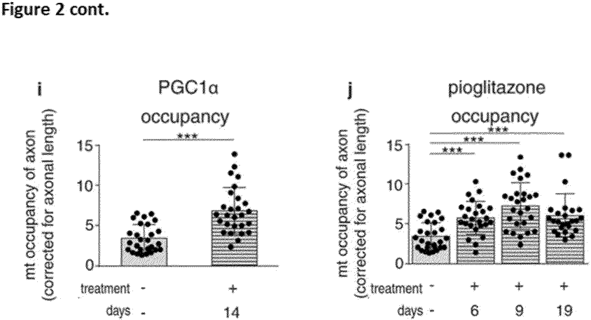

FIG. 2 illustrates enhanced mobilisation of mitochondria from the neuronal cell body to the axon by over-expression of Miro1 and targeting PGC1a/PPAR-y pathway.

a: Following photoconversion of the mEOS2 labeled mitochondria in the proximal axon segment (green to red), time lapse images indicate the anterograde movement of newly transported mitochondria from the unmyelinated DRG neuronal cell body to the proximal axon segment (left to right, see videos, online resource). Over-expression of Mitochondrial Rho GTPase 1 (Miro1) and peroxisome proliferator-activated receptor gamma coactivator 1-alpha (PGC1α) in DRG neurons in culture using lentiviruses as well as exposure of DRG neurons to 2 µM pioglitazone, a thiazolidinedione, enhances anterograde transport of mitochondria, as shown in kymographs and videos, online resource.

b-d: Quantitation of the newly transported mitochondria in the proximal axon segment of unmyelinated axons (green) indicates a significant increase in the number of mitochondria mobilising from the DRG neuronal cell body to the proximal axon segment following over-expression of Miro1 (b) and PGC1α (c) and exposure to pioglitazone (d) compared with untreated DRG neurons (ctl: control). The speed of anterograde moving mitochondria is significantly greater with the over-expression of PGC1α and exposure to pioglitazone. The size of the anterograde moving mitochondria remained unchanged.

Unlike Miro1, both PGC1α over-expression in DRG neurons and exposure to 2 µM pioglitazone significantly increase the size of the stationary mitochondria (red), and the total axonal mitochondrial content.

e-g: PGC1α and Miro1 over-expression as well as the application of pioglitazone to DRG neurons did not significantly alter the anterograde and retrograde transport of lysosomes within unmyelinated axons.

h-j: Similar to unmyelinated axons, Miro1 over-expression does not significantly alter the mitochondrial content within myelinated axons, whilst both PGC1α over-expression and application of pioglitazone significantly increase the axonal mitochondrial content within myelinated axons, in vitro.

Data presented as dot-plot with mean (bar) ± standard deviation (whiskers). *p<0.05, **p<0.01 and ***p<0.001 using Mann-Whitney-U test once Kruskal-Wallis test showed a p<0.05 in multiple subgroup comparisons.

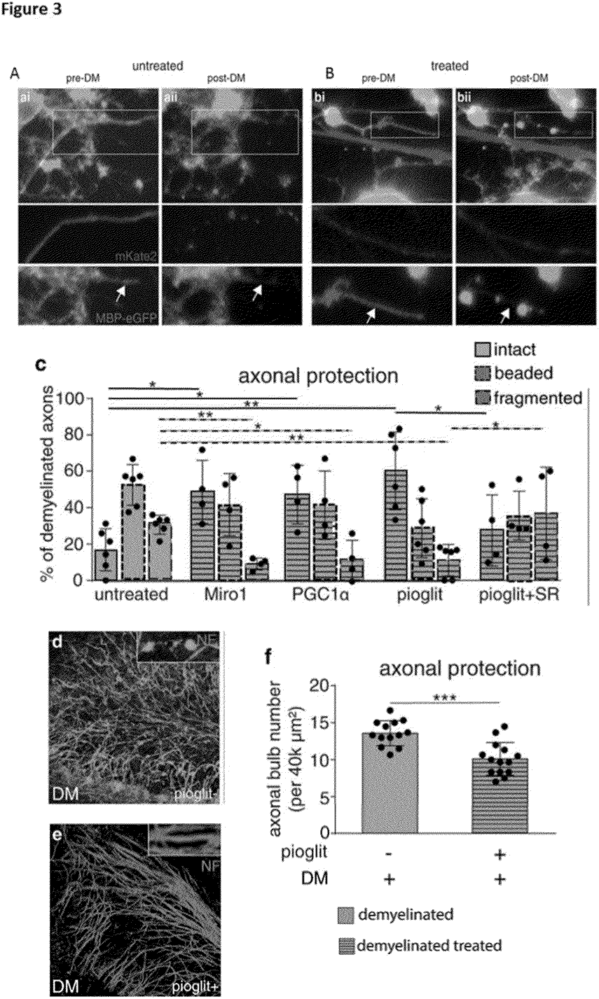

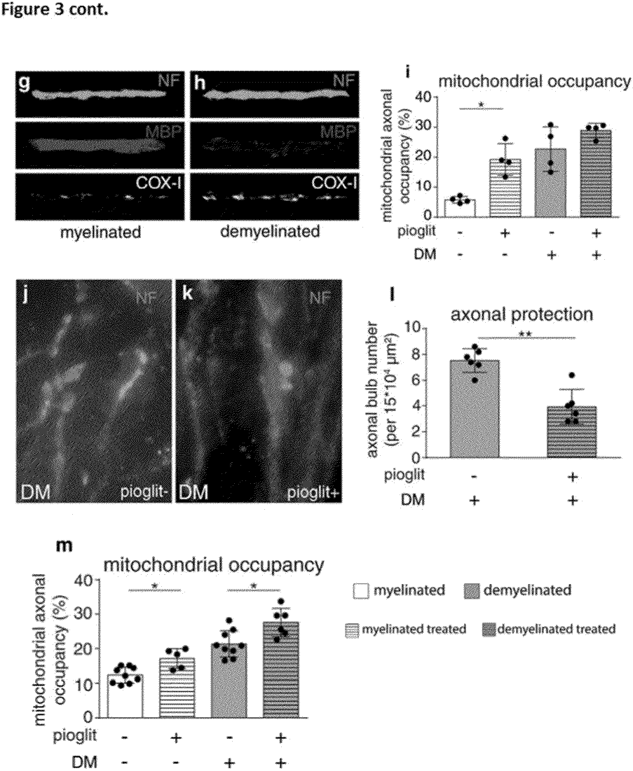

FIG. 3 illustrates enhancement of ARMD in wild type neurons, in vitro and in vivo, protects the acutely demyelinated axons.

a-b: The inventors labelled dorsal root ganglia (DRG) neurons by applying lentivirus-mKate2 (red) to the cell body chamber while MBP produced by oligodendrocytes in the other chamber were labeled using lentivirus M1-M4 (green) (a-b). Prior to demyelination (pre-DM), live images identify myelinated axonal segments in the co-culture chamber (ai and bi, arrows). Following demyelination (post-DM), by exposing to lysolecithin for 2 hours, live imaging shows damaged MBP-positive structures (aii and bii, arrows). The inventors then targeted mitochondria in neurons by applying lentivirus-Miro1, lentivirus-PGC1α and pioglitazone to the neuronal cell body chamber (treated, shown in bi and bii), prior to demyelination. All three manipulations protected the acutely demyelinated axonal segments (b, red) compared with untreated co-culture chambers (a, red).

c: For quantitation, axons were identified as intact (green outlined bar charts), beaded (orange outlined bar charts) and fragmented (red outlined bar charts) based on mKate2 signal, both prior to and following demyelination. Quantitation of axon injury following demyelination revealed a significant decrease in the proportion of demyelinated axons that are fragmented and a significant increase in demyelinated axons that remain intact, when the neuronal cell bodies were exposed to lentivirus-Miro1, lentiviris-PGC1α and pioglitazone (c). PGC1α inhibitor significantly diminished the protective effect of pioglitazone on demyelinated axons. Controls shown were exposed to lentivirus-mKate2. *p<0.05 and **p<0.01 using Mann-Whitney-U test, once Kruskal-Wallis test showed a p<0.05 in multiple subgroup comparisons.

d-i: The inventors detected axon bulbs (d, insert) when cerebellar slice cultures were demyelinated using lysolecithin (0.5 mg/ml) for 17 hours (d). Application of 40 µM pioglitazone to cerebellar slice cultures prior to lysolecithin-induced demyelination significantly decreased the extent of axon bulb formation compared with solvent (DMSO) only controls (f). Axonal mitochondrial content increased upon demyelination of cerebellar slices (g and h), consistent with homeostatic ARMD. The application of pioglitazone significantly increased the mitochondrial content of myelinated axons (i, pioglit+lyso-), compared with untreated cerebellar slices (i, pioglit-lyso-),). ***p<0.001 using Mann-Whitney-U test.

j-m: Demyelination of the spinal cord of wild type mice in vivo, using focal injection of lysolecithin (DM pioglit-) to the dorsal column, increases axon bulb formation (j) compared with non-demyelinated wild type mice (not shown) at 3 days post lesioning. Pioglitazone in diet for 6 weeks (120 mg/Kg diet), prior to focal spinal cord demyelination, significantly decreased axon bulb formation in wild type mice (k, DM pioglit+ and I) compared with controls on chow diet (j, DM pioglit- and I). The inventors did not detect a significant change in DAPI+ cells and Iba1 + cells in lesions with pioglitazone treatment. Mitochondrial content of both myelinated (m, DM-) and demyelinated axons (m, DM+) significantly increased following dietary pioglitazone in wild type mice (m, pioglit+) compared with control (m, pioglit-). Data presented as dot-plot with mean (bar) ± standard deviation (whiskers). *p<0.05 and **p<0.01 using Mann-Whitney-U test. DM: demyelinated. MBP: myelin basic protein. NF: neurofilament. Pioglit: pioglitazone.

FIG. 4 illustrates respiratory deficient neurons are prevalent within dorsal root ganglia in progressive MS and their percentage positively correlates with mitochondrial content, size, number and complex IV deficiency in demyelinated dorsal column axons.

a-b: In progressive MS, dorsal root ganglia (DRG) neurons that lack mitochondrial complex IV and contain complex ll (stained blue by COX/SDH histochemistry, insert in b), termed respiratory deficient, are abundant (b) compared with controls (a). The majority of neurons show intact complex IV in controls (stained brown, insert in a).

c: Quantitation of DRG in progressive MS identified significantly more respiratory deficient neurons in lumbar DRGs compared with controls (p<0.0001). Respiratory deficient neurons tended to be more prevalent in lumbar DRG than cervical DRG [the broken lines (c) indicate data from the same case]. In Parkinson’s disease the mean respiratory deficient neurons in lumbar DRG is 28.77% (SD = 13.66, n=3) and the mean is 33.51% (SD = 13.66, n=2) in motor neuron disease (MND) lumbar DRG (not shown). Data presented as dot-plot with mean (bar) ± standard deviation (whiskers). ****P<0.0001 using Mann-Whitney-U test and Kruskal-Wallis test showed a p<0.0001 in multiple subgroup comparisons.

d: Chronic spinal cord lesions in dorsal columns, at the corresponding dorsal root entry zone, of six progressive MS cases enabled the impact of respiratory deficient neurons on the mitochondrial parameters of demyelinated axons to be assessed.

e: Mitochondrial content in demyelinated dorsal column axons at the root entry zone, as a percentage of axon area, correlated significantly (p=0.004, r²=0.904) and positively with the percentage of respiratory deficient proprioceptive neurons in lumbar DRG in progressive MS (ei).

Average size of mitochondria within demyelinated dorsal column axons correlated significantly (p=0.044, r²=0.677) and positively with the percentage of respiratory deficient proprioceptive neurons in lumbar DRG in progressive MS (eii).

Average number of mitochondria within demyelinated dorsal column axons at the root entry zone correlated significantly (p=0.026, r²=0.747) and positively with the percentage of respiratory deficient proprioceptive neurons in lumbar DRG in progressive MS (eiii).

As expected, the percentage of the axon occupied by mitochondria that lacked complex IV subunit-l correlated significantly (p=0.023, r²=0.763) with the percentage of respiratory deficient proprioceptive neurons in lumbar DRG in progressive MS (eiv).

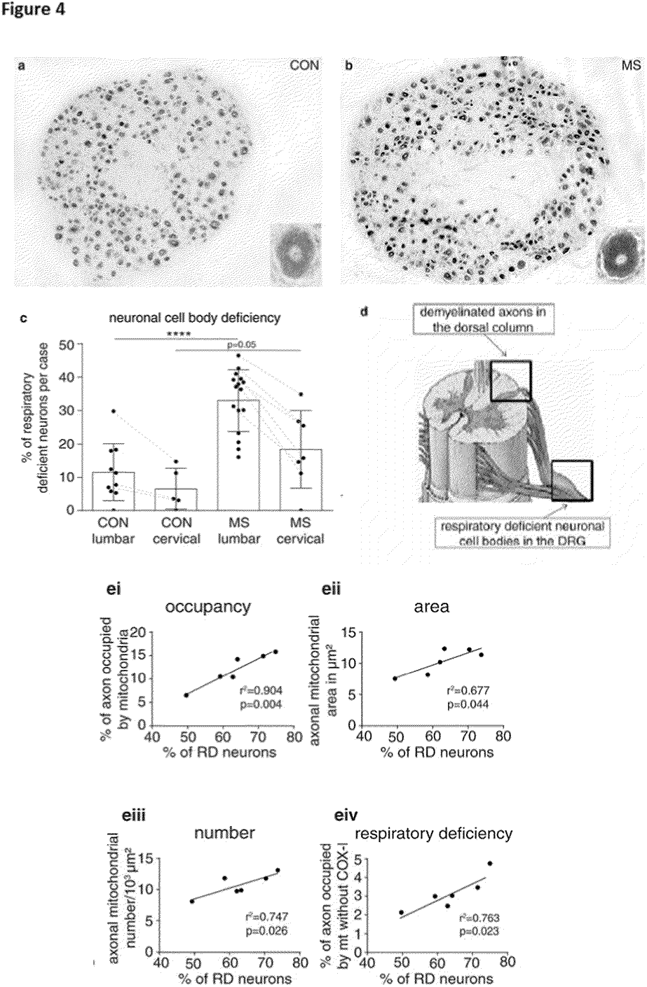

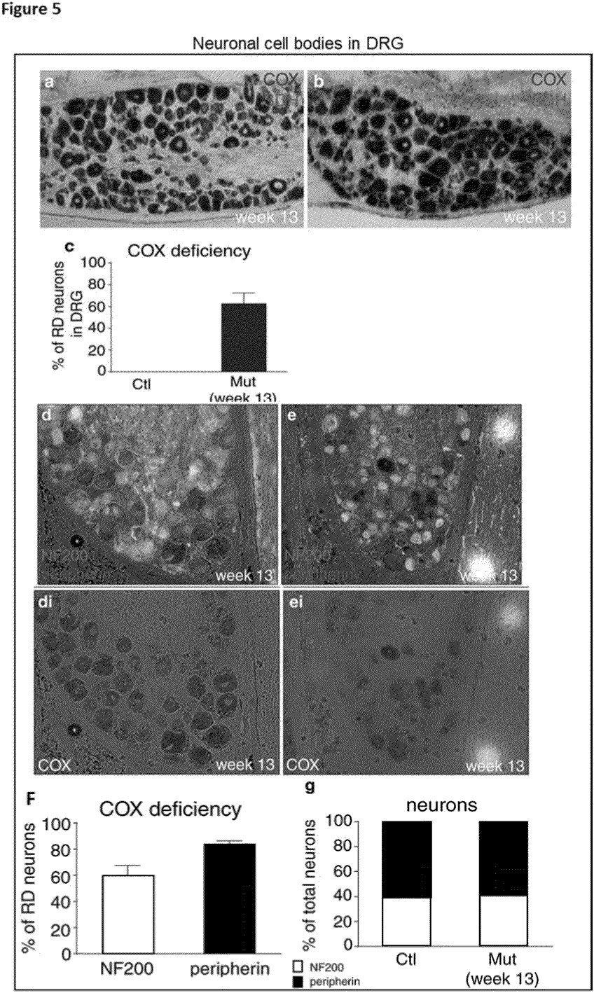

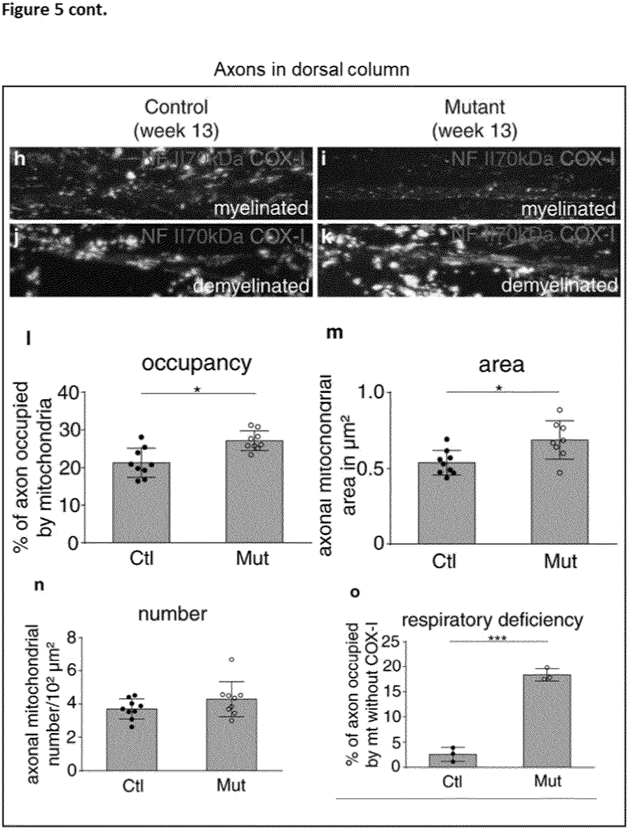

FIG. 5 illustrates modeling the complex IV deficient DRG neurons and recapitulating mitochondrial changes within demyelinated axons in progressive MS, in vivo.

a-c: DRG neuron-specific inducible knockout mice that lack protoheme IX farnesyltransferase [subunit 10 of complex IV (COX10), termed COX10Adv mutants) contain complex IV deficient DRG neurons (b), which are stained blue by the sequential COX/SDH histochemistry assay. DRG neurons with intact complex IV are stained brown in both wild type mice and COX10Adv mutants (a and b). The quantitation identified approximately 59% of the DRG neurons as respiratory deficient in COX10Adv mice (c, n=3) compared with none in controls (c, n=3).

d-f: Sequential COX histochemistry and immunofluorescent labeling method, as previously described, identifies both proprioceptive (NF200+peripherin-, in green) and nociceptive neurons (NF200-peripherin+, in red) in DRG that are respiratory deficient (lack of or decrease in intensity of brown staining following COX histochemistry, shown in grey scale images in di and ei) in COX10Adv mutants (ei), unlike wild type mice (di). The quantitation of complex IV within immunofluorescently labeled proprioceptive and nociceptive neurons, using densitometry, identifies approximately 59.33 +/- 8.14 % and 83.33 +/- 3.05% of proprioceptive and nociceptive neurons, respectively, in COX10Adv mutants to be lacking complex IV (f). Complex IV deficient neurons, judged by densitometric analysis of COX histochemistry, are not present within DRG from wild type mice (a).

g: Quantitation of proprioceptive (NF200-positive in green) and nociceptive (peripherin-positive in red) DRG neurons from 13 week old wild type (n=3) and COX10Adv mutants (n=3) shows similar proportion of proprioceptive and nociceptive neurons in both groups.

h-k: In focal lysolecithin-induced lesions of the dorsal columns, demyelinated axons (NF in blue) contain more mitochondria (j and k) than myelinated axons (h and i), when mitochondria are identified with complex ll 70 KDa subunit (red) labeling, in both wild type (h and j) and COX10Adv mutants (i and k). As expected, complex IV subunit-l (green) is lacking within axonal mitochondria in COX10Adv mutants (i and k).

l-o: Quantitation of mitochondria within demyelinated dorsal column axons from wild type mice and COX10Adv mutants shows a significantly greater mitochondrial occupancy (l) and mitochondrial size (m) as well as extent of mitochondria lacking complex IV subunit-l (o) in experimentally demyelinated COX10Adv mutants compared wild type mice. These findings are concordant with the positive correlations that the inventors observed between the extent of respiratory deficient proprioceptive neurons and the mitochondrial parameters within demyelinated dorsal column axons at the root entry zone in progressive MS (shown in FIGS. 5E-F). *p<0.05 and ***p<0.001 using Mann-Whitney-U test.

Data presented as dot-plot with mean (bar) ± standard deviation (whiskers).

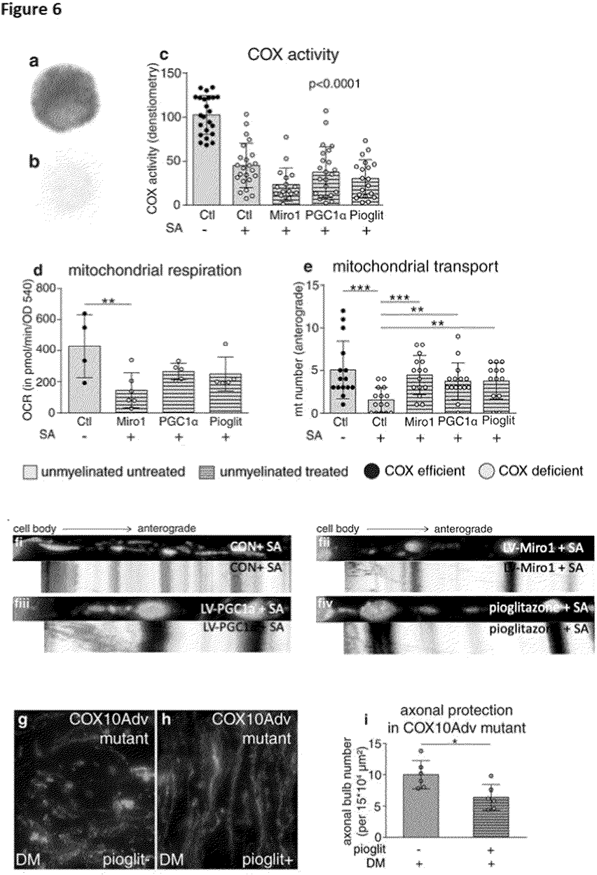

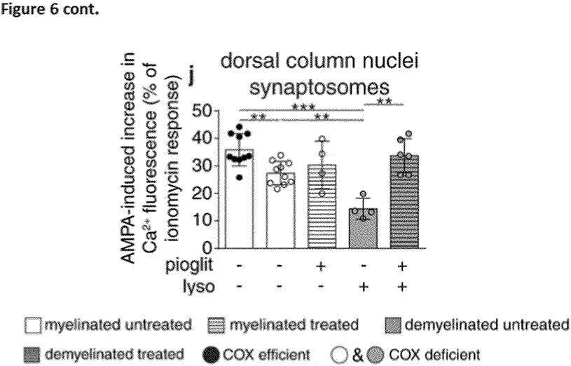

FIG. 6 illustrates enhancement of ARMD in complex IV deficient neurons protects the extremely vulnerable acutely demyelinated axons.

a-c: To model complex IV deficiency in vitro, the inventors pharmacologically inhibited it using sodium azide (SA, at 100 µM for 16 hours), which significantly decreases complex IV activity in wild type DRG neurons (b and c), as expected, compared with controls (a and c). The inhibition of complex IV by SA is similarly effective in DRG neurons, where Miro1 and PGC1α are over-expressed, using lentiviruses, and when exposed to pioglitazone (c). Controls shown were exposed to lentivirus-mEOS2. Solvent (DMSO) only controls for pioglitazone treatment did not show a significant effect compared with lentivirus-mEOS2 controls without DMSO (not shown). ***p<0.001 using Mann-Whitney-U test and Kruskal-Wallis test showed a p<0.05 in multiple subgroup comparisons.

d: Mitochondrial respiration significantly decreases when DRG neurons that are over-expressing Miro1 are exposed to SA (100 µM for 16 hours) (d). Although mitochondrial respiration tends to decrease when DRG neurons that are over-expressing PGC1α and treated with pioglitazone are exposed to SA, the decrease is not statistically significant (d), despite the significant inhibition of complex IV activity (c). **p<0.01 using Mann-Whitney-U test and Kruskal-Wallis test showed a p<0.05 in multiple subgroup comparisons.

e-f: As expected, the number of mitochondria mobilising from the DRG neuronal cell body to the axon significantly decreases following inhibition of complex IV by SA (100 µM for 16 hours) compared with DRG neurons not exposed to SA. In contrast, SA does not significantly decrease the anterograde movement of mitochondria from the cell body to the axon in DRG neurons where Miro1 and PGC1α are over-expressed, using lentiviruses, and when DRG neurons are treated with pioglitazone (e). Kymographs show the improvement in the number of mitochondria mobilising from the complex IV deficient neuronal cell body to the axon, which is mediated by Miro1 (fii), PGC1α (fiii) and pioglitazone (fiv). **p<0.01 and ***p<0.001 using Mann-Whitney-U test and Kruskal-Wallis test showed a p<0.05 in multiple subgroup comparisons.

g-i: When dorsal column axons are demyelinated (g, DM) by focal lysolecithin injections to the dorsal columns of COX10Adv mutant mice with complex IV deficient DRG neurons, there is an abundance of axon bulbs at 3 days post lesioning (g and i). Pioglitazone in diet for 6 weeks (120 mg/Kg diet), prior to focal dorsal column demyelination, significantly decreased the axon bulb formation within the demyelinated area in COX10Adv mutant mice (h, DM pioglit+) compared with untreated COX10Adv mutants (g, DM pioglit-) after focal demyelination of dorsal columns (i). *p<0.05 and **p<0.01 using Mann-Whitney-U test.

j: Ca²⁺ fluorescence responses of dorsal column nuclei (DCN) synaptoneurosomes evoked by AMPA (40 µM) in the presence of cyclothiazide (20 µM) were significantly attenuated in COX10Adv mutant mice compared to wild type and the impairment was exacerbated in mice with dorsal column demyelination 3 days previously. Dietary treatment with pioglitazone for 6 weeks beforehand fully rescued the functional deficits. Responses of non-pioglitazone controls (treated with DMSO vehicle) showed no significant difference from naive controls (**p<0.01, ***p<0.001; One-Way ANOVA with Tukey’s test, n=4-10).

COX10Adv: Inducible and DRG neuron-specific (Adv: advillin) knock out of complex IV subunit 10 (COX10) in mice, with complex IV deficient DRG neurons.

Data presented as dot-plot with mean (bar) ± standard deviation (whiskers). DM: demyelinated. Pioglit: pioglitazone. SA: sodium azide, complex IV inhibitor. *p<0.05, **p<0.01 and ***p<0.001.

FIG. 7 illustrates schematic of the novel neuroprotective strategy to preserve acutely demyelinated axons by increasing the mobilisation of mitochondria from the neuronal cell body to the axon.

a-g: Energy efficiency offered by myelination is reflected by a decrease in mitochondrial content in myelinated axons compared with unmyelinated axons, which is elegantly illustrated by the healthy optic nerve (a-b) [5]. Immediately following myelin loss, the inventors show that mitochondria increasingly mobilise from the neuronal cell body to the acutely demyelinated axons, leading to a gradual increase in the axonal mitochondrial content (c and f), which the inventors term axonal response of mitochondria to demyelination (ARMD). ARMD is a homeostatic phenomenon that attempts to increase the energy producing capacity of the acutely demyelinated axons (hom-ARMD). However, hom-ARMD is not sufficient to protect the acutely demyelinated axon, which undergoes transection within days of myelin loss and where myelin debris is still evident (c). During the time that is required by hom-ARMD to increase the mitochondrial content of the demyelinated axons (days), towards the level of the unmyelinated axons, the acutely demyelinated axons are particularly vulnerable and degenerate (c and f). Increased mobilisation of mitochondria from the neuronal cell body to the axons, which results in an enhanced ARMD (enh-ARMD) in both wild type neurons and those with complex IV deficiency, protects the vulnerable acutely demyelinated axons (d and g). This novel neuroprotective strategy is crucial to protect the acutely demyelinated axons so that, subsequently, neurorestorative therapy like remyelination can be implemented in demyelinating disorders.

ARMD: axonal response of mitochondria to demyelination. DM: demyelinated axon. Enh-ARMD: enhanced ARMD. Hom-ARMD: homeostatic ARMD. M: myelinated axon. UM: unmyelinated axon.

FIG. 8 illustrates Lysolecithin does not impact mitochondrial dynamics within dysmyelinated axon in Shiverer mice wherein A-D: Time-lapse imaging of Purkinje cell mitochondria labeled with photoconvertible mEOS2 (green in unconverted state) in cerebellar slice cultures show the presence of newly transported mitochondria (green) within the dysmyelinated axons in Shiverer mice (A). Kymorgraph shows both anterograde (left to right) and retrograde movement of newly transported mitochondria within the dysmyelinated axons (C). Similar findings were noted when cerebellar slices from Shiverer mice were exposed to lysolecithin (B and D); E-H: Quantitation of the newly transported mitochondria in the chronically dysmyelinated axons shows that lysolecithin does not significantly impact axonal mitochondrial dynamics.

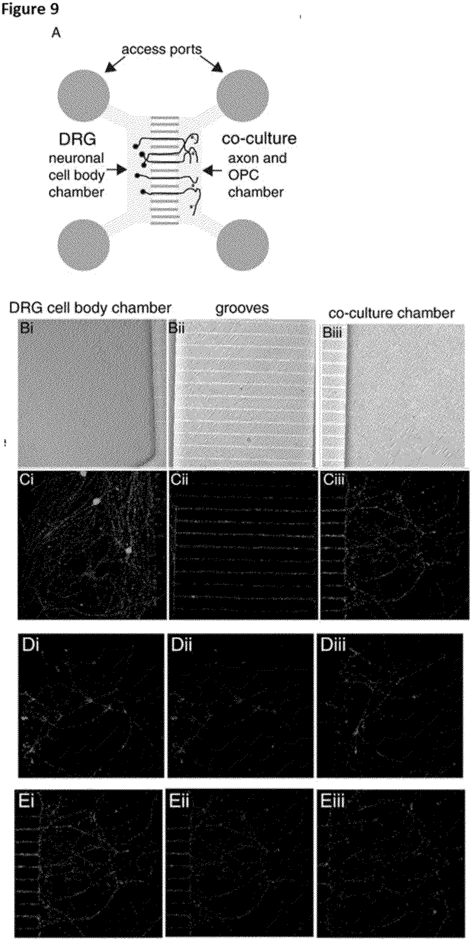

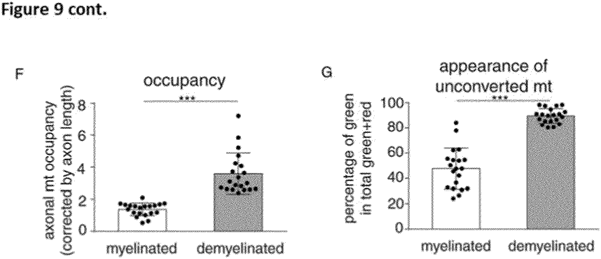

FIG. 9 illustrates unconverted mitochondria more abundant in axons following demyelination in microfluidic chambers; wherein A-l: Application of lentivirus-mitochondria-targeted mEOS2 to dorsal root ganglia (DRG) neurons in the neuronal cell body compartment of microfluidic chambers (A, B and G) labels mitochondria in DRG neurons, including mitochondria within the axons that traverse the grooves between the chambers (A, C and H) and enter the co-culture chamber (A, D and I). Oligodendrocyte progenitor cells (OPCs) in the co-culture chamber (A, D and I) myelinate axons, which are visualized using SCoRE (E) and confirmed using immunofluorescent labeling of myelin basic protein [MBP, F (blue)] and neurofilament [NF, F (red)]; J-K: Photoconversion of mEOS2 labeled mitochondria in the co-culture chamber (Ji and Jii) allows mitochondria that subsequently enter the photoconverted region to be assessed in myelinated axon segments (green in Jiii) as well as demyelinated axons [(green in Kiii following photoconversion of green (Ki) to red (Kii)]; L-N: Lysolecithin-induced demyelination led to a significant increase in mitochondrial content within axons in the co-culture chamber, 16 hours post-lysolecithin exposure (L, when green and red channels are merged), as previously reported, in vivo. Furthermore, the area of green labeled mitochondria in axons as a percentage of total area of mitochondria (when green and red channels are merged) as well as photoconverted mitochondria (red) is significantly greater following exposure to lysolecithin (M and N, respectively), indicating the greater movement of mitochondria from outside the photoconverted regions to the photoconverted region, following demyelination

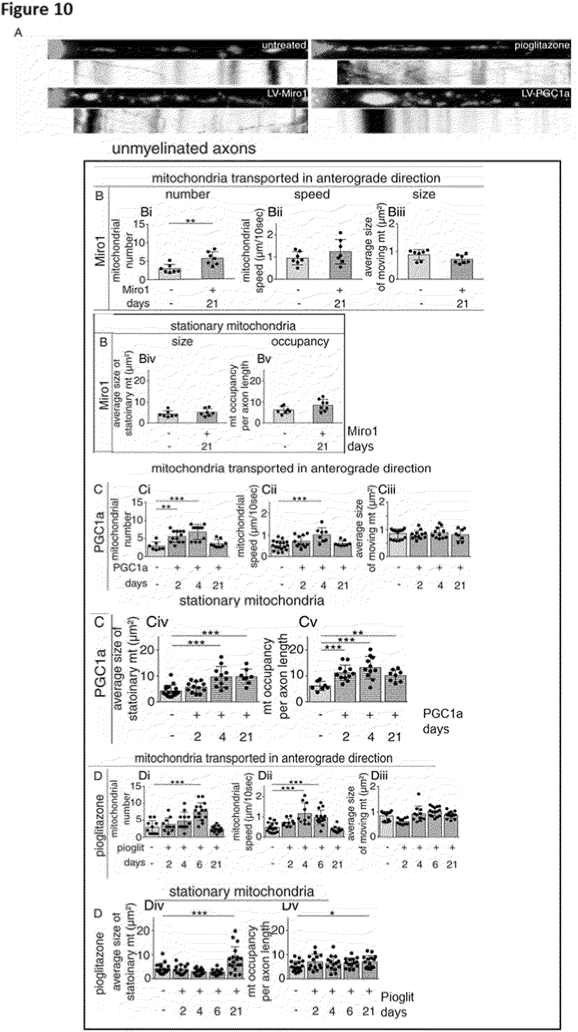

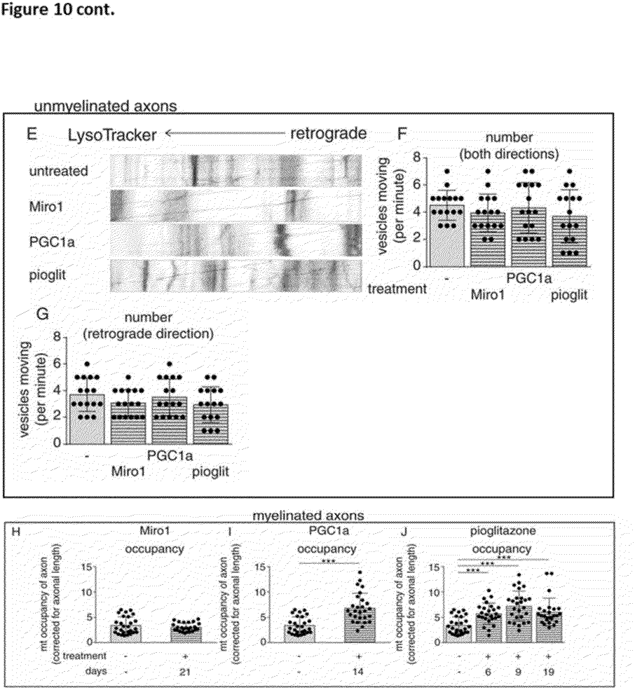

FIG. 10 illustrates enhancement of mitochondrial movement from the neuronal cell body to the axon and increasing the mitochondrial content in axons by targeting PGC1a/PPAR-y pathway wherein A: Over-expression of Mitochondrial Rho GTPase 1 (Miro1) and peroxisome proliferator-activated receptor gamma coactivator 1-alpha (PGC1-alpha) in DRG neurons in culture using lentiviruses as well as exposure of DRG neurons to 2 µM pioglitazone, a thiazolidinedione, enable the impact of enhancing anterograde transport of mitochondria (Miro1) as well as mitochondrial biogenesis (PGC1-alpha and pioglitazone) to be assessed. Following photoconversion of the mEOS2 labeled mitochondria in the proximal axon segment (green to red), kymographs of the green channel generated from time lapse images indicate the anterograde movement of newly transported mitochondria from the unmyelinated DRG neuronal cell body to the proximal axon segment (left to right, see videos); B: Quantitation of the newly transported mitochondria in the proximal axon segment of unmyelinated axons (green), in time lapse images following photoconversion, indicates a significant increase in the number of mitochondria undergoing anterograde transport from the DRG neuronal cell body to the proximal axon segment following over-expression of Miro1 and PGC1-alpha as well as exposure to pioglitazone compared with untreated DRG neurons (ctl: control). The speed of anterograde moving mitochondria was significantly greater with the over-expression of PGC1-alpha and exposure to pioglitazone and not significantly different following over expression of Miro1. The above manipulations did not significantly change the size of the anterograde moving mitochondria - Both PGC1-alpha over-expression in DRG neurons and exposure to 2 µM pioglitazone significantly increased the size of the stationary mitochondria, which were indicated by the photoconverted mitochondria (red) that did not move during time-lapse imaging, as well as the total mitochondrial content within the unmyelinated axons. In contrast, Miro1 did not alter the size of stationary mitochondria and the total mitochondrial content; C: PGC1-alpha and Miro1 over-expression as well as the application of pioglitazone to DRG neurons did not significantly alter the transport of lysosomes within unmyelinated axons [in both directions and the dominant direction, which is indicative of retrograde movement (right to left) of lysosomes]; D: Co-cultures of DRG neurons and oligodendrocyte progenitor cells and over-expression of Miro1 and PGC1-alpha in neurons as well as exposure of the co-cultures to pioglitazone enable the impact of the three manipulations on mitochondria in myelinated axons (SCoRE positive) to be determined. Miro1 over-expression did not significantly alter the mitochondrial content within myelinated axons. Similar to the findings in unmyelinated axons both PGC1-alpha over-expression and application of pioglitazone significantly increase the axonal mitochondrial content within myelinated axons, in vitro.

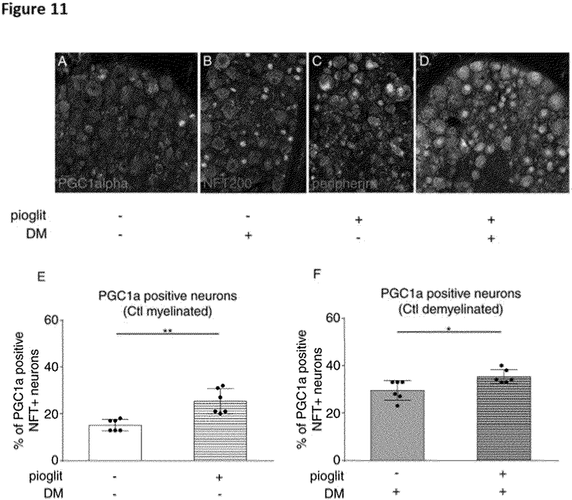

FIG. 11 illustrates PGC1a positive nuclei within DRG neurons in wild type mice increased significantly following focal demyelination of the dorsal columns and administration of pioglitazone in diet wherein A-F: PGC1-alpha positive neuronal nuclei (green) are relatively infrequent within DRG neurons from wild type mice on chow diet (A, untreated and not demyelinated). Pioglitazone in diet for 6 weeks significantly increases the percentage of DRG neurons with PGC1-alpha positive nuclei (C and E) in wild type mice (neg) compared with mice on chow diet (A and E). Demyelination of the dorsal columns of untreated wild type mice (neg), using lysolecithin, increases in PGC1-alpha positive DRG neuronal nuclei (B) compared with untreated and non-demyelinated wild type animals (A, E and F). Demyelination of the dorsal columns of pioglitazone treated wild type mice (neg), using lysolecithin, further increases in PGC1-alpha positive DRG neuronal nuclei (D and F) compared with untreated and demyelinated wild type animals (B and F).

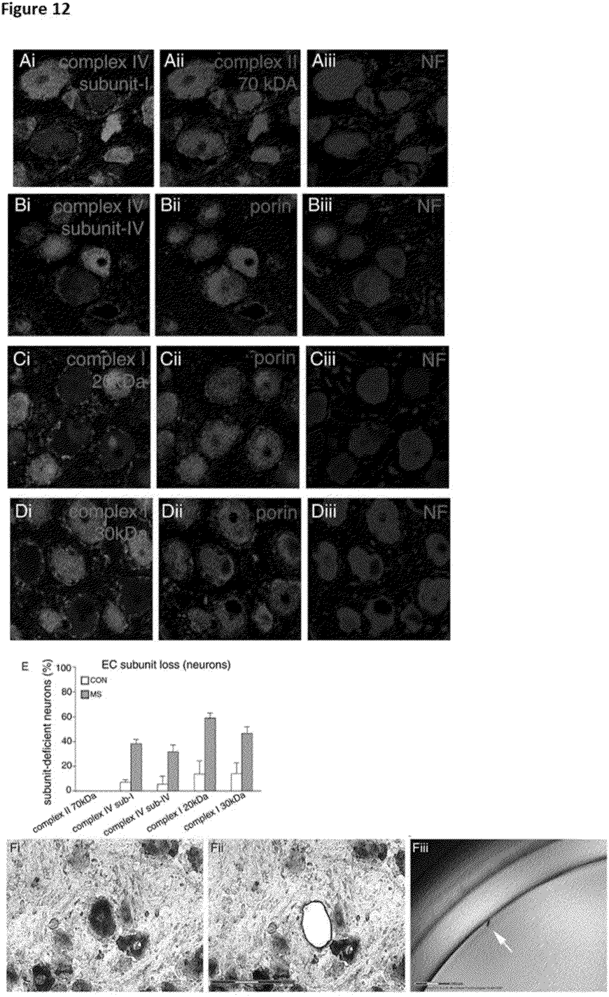

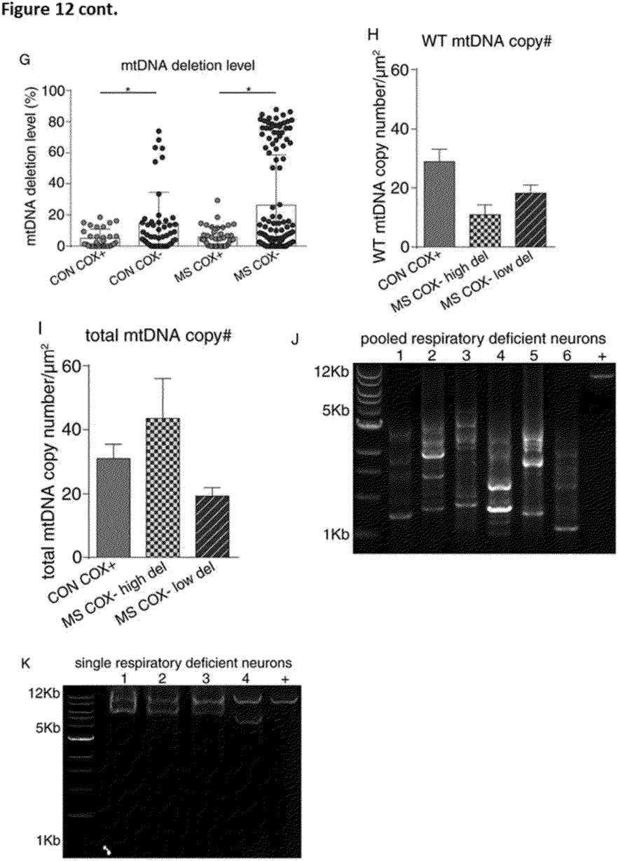

FIG. 12 illustrates respiratory deficient DRG neurons in progressive MS lack mitochondrial respiratory chain complex subunits and harbor clonally expanded mitochondrial DNA deletions at a high heteroplasmy level wherein A-D: Immunofluorescent labeling of mitochondrial respiratory chain complex subunits in DRG in progressive MS - Complex IV subunit-l (Ai, green), complex ll 70 kDa (Aii, red) and total neurofilament (Aiii, blue) triple labeling identified a subset of neurons with the complex ll subunit and lacking the mitochondrial DNA encoded complex IV subunit-l in MS DRG (A, arrowheads) - Complex IV subunit-IV (Bi, green), porin (Bii, red, a voltage gated anion channel expressed on all mitochondria) and total neurofilament (Biii, blue) triple labeling identified a subset of neurons lacking the above complex IV subunit in MS DRG (B, arrowheads). The complex IV subunit-IV labeling was detected in the peri-neuronal region surround the subunit deficient neurons - Complex l 20 kDa (Ci, green), porin (Cii, red) and total neurofilament (Ciii, blue) triple labeling identified a subset of neurons lacking the above complex I subunit in MS DRG (C, arrowheads). As with the complex IV subunits, complex I 20 kDa labeling was detected in the peri-neuronal region surround the subunit deficient neurons - Complex l 30 kDa (Di, green), porin (Dii, red) and total neurofilament (Diii, blue) triple labeling identified a subset of neurons lacking the complex l subunit in MS DRG (D, arrowheads). As with the complex IV subunits and complex l 20 kDa, complex l 30 kDa labeling was detected in the peri-neuronal region surround the subunit deficient neurons; E: Quantitation of DRG neurons lacking the subunits identified significantly greater percentage of neurons with loss of subunits in progressive MS than controls; F: Respiratory deficient neurons (blue) and neurons with intact complex IV (brown) in cryostat sections placed on membrane slides (Fi). Following laser microdissection (Fii), the captured single neuron is apparent in the cap of sterile eppendorf tube (Fiii, arrow); G: When the level of mitochondrial DNA (mtDNA) deletion was determined at a single cell level by MTND1/MTND4 real time PCR, the mean percentage of mtDNA deletion was significantly greater in respiratory deficient neurons (blue, COX negative) than those with intact complex IV activity (brown, COX positive) in both controls and multiple sclerosis (MS). The high level of mtDNA deletion (>50% threshold consistently reported) adequately explained the biochemical defect in 6 out of the 48 respiratory deficient neurons in control and 40 out of the 128 in MS; H-l: As expected respiratory deficient neurons containing high heteroplasmy level of mtDNA deletion contained significantly less wild type mtDNA (H). The total mtDNA copy number did not significantly differ in respiratory deficient neurons from MS cases (MS COX negative) compared with neurons from control (CON, mostly consists of cells with intact complex IV activity). However, the respiratory deficient neurons harboring high level mtDNA deletion in MS contained significantly more total mtDNA copies (MTND1) than those with low level mtDNA deletion in MS as well as the neurons from controls (l) ; J-K: To further characterize the mtDNA deletions in MS, long range PCR was performed on pooled respiratory deficient neurons (n=20 cells per lane), where multiple deletions of mtDNA were detected (J). In contrast to the multiple mtDNA deletions within pooled respiratory deficient DRG neurons, pooled neurons with intact complex IV activity (n=20 per lane) showed relatively few mtDNA deletions (not shown). When long range PCR was performed on single respiratory deficient neurons, mostly one mtDNA deletion was detected within single cells, which is consistent with clonal expansion of mtDNA deletion (K).

FIG. 13 illustrates disease models lack respiratory deficient cells in the brain, spinal cord and dorsal root ganglia wherein A-C illustrates the lack of cells deficient in complex IV and with intact complex ll (respiratory-deficient, identified by sequential COX/SDH assay) in the spinal cord from Biozzi ABH EAE mice at acute (A), relapsing (B) and chronic stages (C); D-L: Respiratory deficient choroid plexus epithelial cells that lack complex IV and contain complex ll (D, stained blue, arrow) were infrequently found at the chronic stage of Biozzi ABH EAE mice (6 in 10 animals). The sequential COX histochemistry (Ei-Gi) and immunofluorescent labeling of complex ll 70 kDa (E-G, red) and complex IV subunit-l (Eiii-Giii, green) assay identified complex IV-deficient mitochondria, as previously reported, in spinal cord regions with inflammation in Biozzi ABH EAE. Complex VI deficient mitochondria contain both the subunits in the spinal cord (asterisks). The respiratory-deficient choroid plexus epithelial cells, however, contain complex ll 70 kDa (Hii, arrowhead) and lack complex IV subunit-l (Hiii, arrowhead), as reported in progressive MS. The merged image of complex IV activity (Hi) and immunofluorescent labeling of subunits (Hii and Hiii) is shown in L (arrowhead showing the respiratory-deficient cells). The regions of the white matter that lacked complex IV activity despite the presence of complex IV subunit-l (indicated by asterisk in E-G) correspond to regions containing inflammatory infiltrates (asterisks) in acute (I), relapsing (J) and chronic phases (K), as shown by H&E staining of serial sections; M-P: These regions with inflammation showed immunoreactivity for inducible nitric oxide synthase (iNOS) in acute (M), relapsing (N) and chronic phases (O) compared with control tissue (P). Scale bar indicates 60 µm in all except D. Scale bar in D indicates 50 µm.

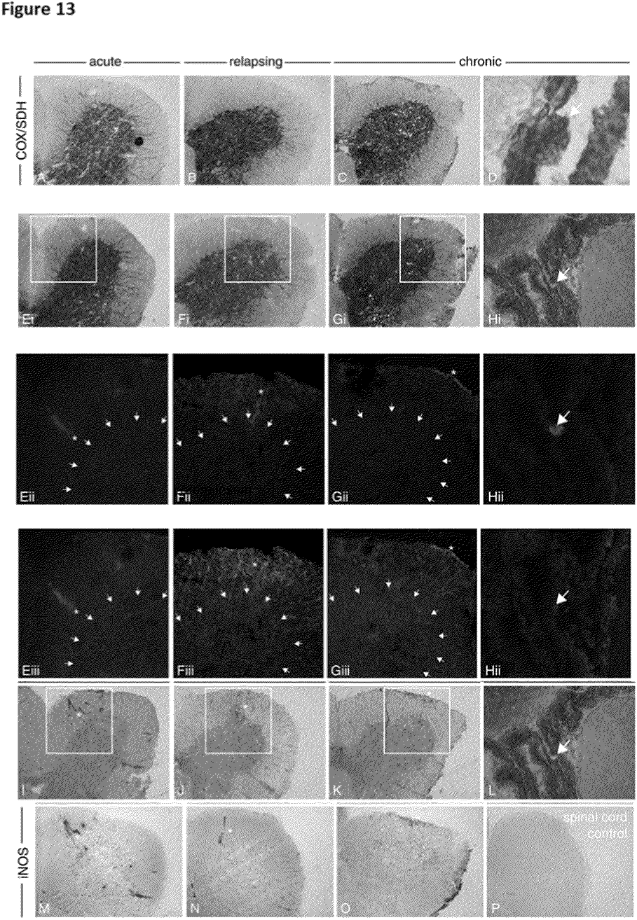

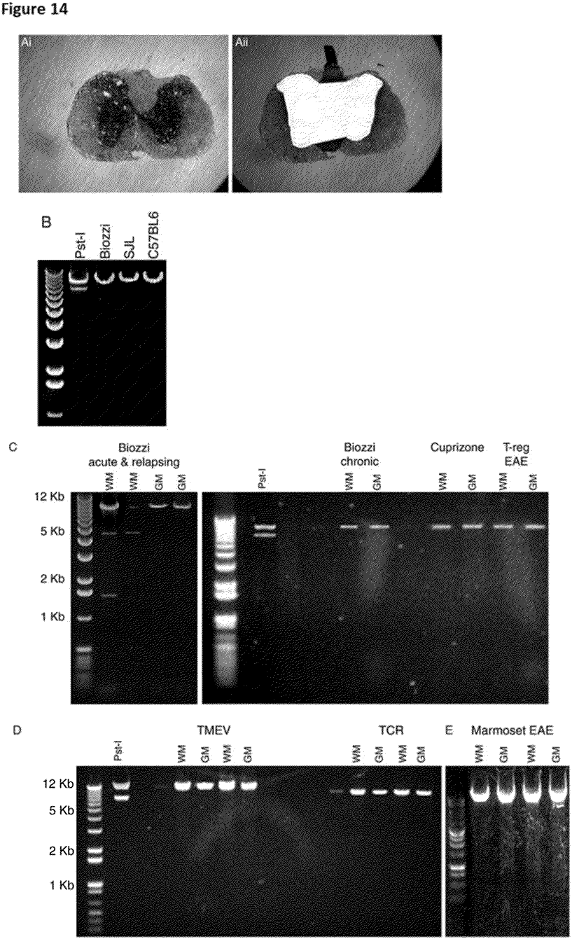

FIG. 14 illustrates mitochondrial DNA deletions are rarely detected within the CNS in the disease models wherein A-B: The spinal cord grey matter (GM) in cryosections with inflammation was microdissected, as evident in pre- and post- laser capture images (A). Pstl tissue shows a mitochondrial DNA deletion (lower band), as a positive control (B). Only the wild type bands are seen in control Biozzi ABH, SJL and C57 mouse spinal cord grey matter (B); C-E: Acute and relapsing phases of Biozzi EAE showed mtDNA deletions in (C, arrowheads) the white matter (WM), which were absent at the chronic stage and in the grey matter. Mitochondrial DNA deletions were not detected in the cuprizone model and T-reg depleted EAE (C) as well as Theiler’s murine encephalomyelitis (TMEV) and human T cell receptor (TCR) transgenic mice with spontaneous EAE (D) as well as marmoset EAE (E).

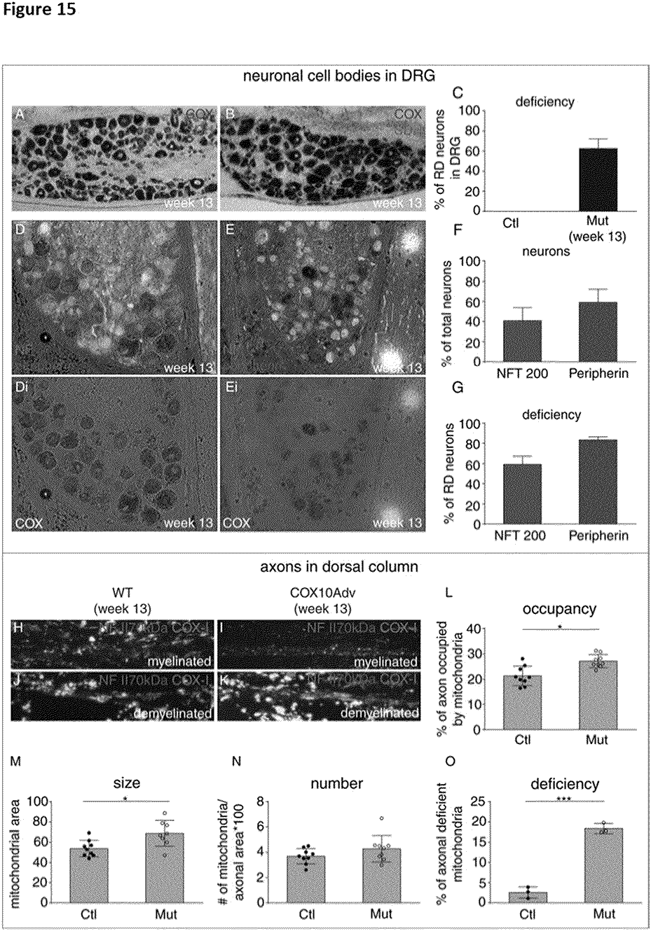

FIG. 15 illustrates modeling the complex IV deficient DRG neurons and mitochondrial changes within demyelinated axons in progressive MS, in vivo wherein A-C: DRG neuron-specific inducible knockout mice that lack protoheme IX farnesyltransferase [subunit 10 of complex IV (COX10), termed COX10Adv mutants) contain complex IV deficient DRG neurons (B), which are stained blue by the sequential COX/SDH histochemistry assay. DRG neurons with intact complex IV are stained brown in both wild type mice and COX10Adv mutants (A and B). The quantitation identified approximately 59% of the DRG neurons as respiratory deficient in COX10Adv mice (C, n=3) compared with none in controls (C, n=3); D-F: Sequential COX histochemistry and immunofluorescent labeling method, as previously described, identifies both proprioceptive (NF200+peripherin-, in green) and nociceptive neurons (NF200-peripherin+, in red) in DRG that are respiratory deficient (lack of or decrease in intensity of brown staining following COX histochemistry, shown in grey scale images in Di and Ei) in COX10Adv mutants (Ei), unlike wild type mice (Di). The quantitation of complex IV within immunofluorescently labeled proprioceptive and nociceptive neurons, using densitometry, identifies approximately 59.33 +/- 8.14 % and 83.33 +/- 3.05% of proprioceptive and nociceptive neurons, respectively, in COX10Adv mutants to be lacking complex IV (F). Complex IV deficient neurons, judged by densitometric analysis of COX histochemistry, were not present within DRG from wild type mice (F); G: Quantitation of proprioceptive and nociceptive DRG neurons from 13 week old wild type (n=3) and COX10Adv mutants (n=3) shows comparable number of neurons in both groups with similar proportion of proprioceptive (green) and nociceptive (red) neurons; H-K: In focal lysolecithin-induced lesions of the dorsal columns, demyelinated axons (NF in blue) contain more mitochondria (J and K) than myelinated axons (H and l), when mitochondria are identified with complex ll 70 KDa subunit (red) labeling, in both wild type (H and J) and COX10Adv mutants (l and K). As expected, complex IV subunit-l (green) was lacking within axonal mitochondria in COX10Adv mutants (l and K); L-O: Quantitation of mitochondria within demyelinated dorsal column axons from wild type mice and COX10Adv mutants shows a significantly greater mitochondrial occupany (L) and mitochondrial size (M) as well as extent of mitochondria lacking complex IV subunit-l (O) in experimentally demyelinated COX10Adv mutants compared wild type mice. These findings are concordant with the positive correlations that the inventors observed between the extent of respiratory deficient proprioceptive neurons and the mitochondrial parameters within demyelinated dorsal column axons at the root entry zone in progressive MS (shown in FIGS. 5E-F).

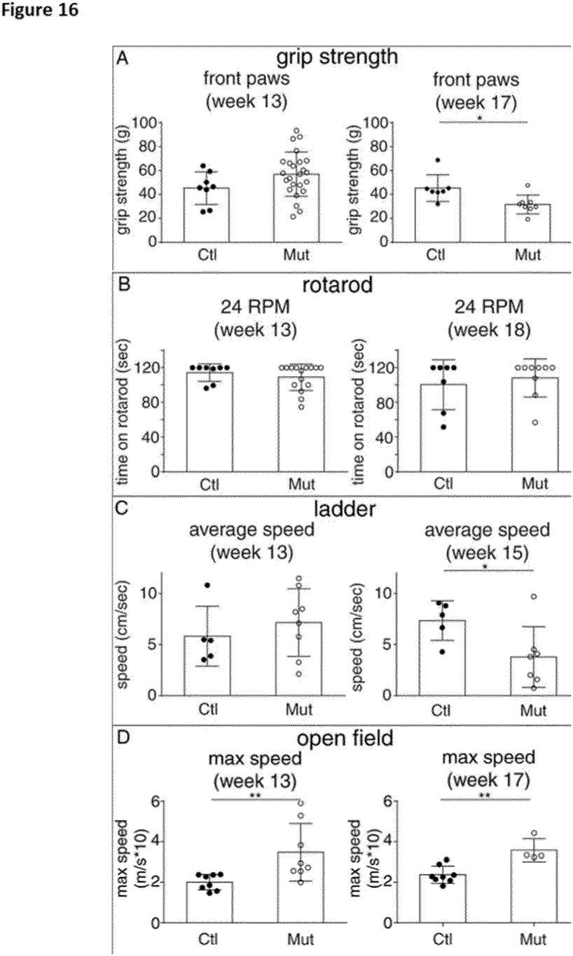

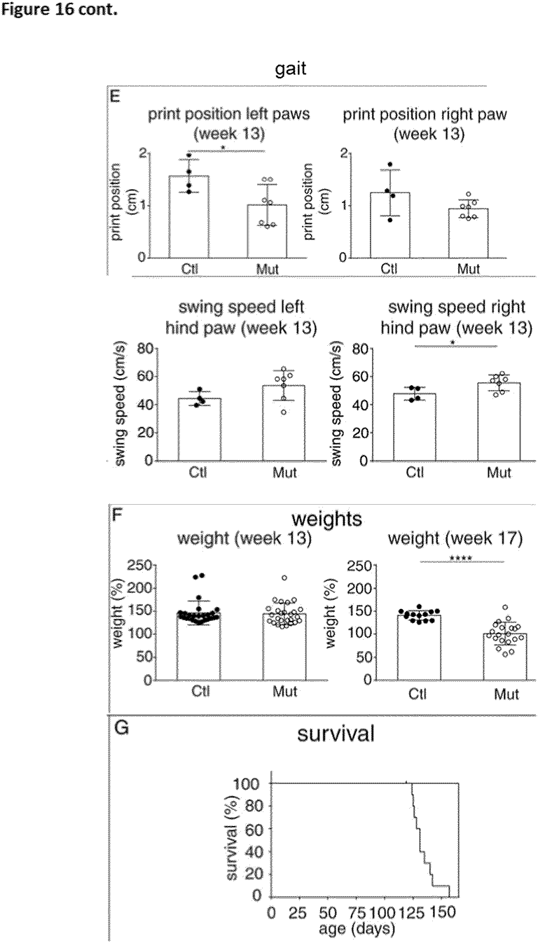

FIG. 16 illustrates behavioural testing reveals a subtle clinical phenotype of COX10Adv mutant mice when experimental demyelination was carried out wherein A-D: 13 weeks following the completion of tamoxifen gavaging, the time point at which focal experimental demyelination of the spinal cord was carried out, COX10Adv mutant mice do not show a significant difference in front paw grip strength (A), rotarod performance at 24 rpm (B), performance on horizontal ladder testing (C) and all but one parameter on open filed testing (D). Maximum speed on open field testing was significantly greater in the COX10Adv mutant mice compared with control mice (D); E: Detailed gait analysis using CatWalk system showed a significantly lower print position of left paws and a significantly greater swing speed of right hind paw at 13 weeks following completion of tamoxifen in COX10Adv mutant mice compared with control mice, which are the only significant behavioural findings in COX10Adv mutant mice at 13 weeks post-tamoxifen, out of 20 parameters. At later time points, the inventors detected significant changes in a number of behavioural parameters. Grip strength of front paws was significantly lower at 17 weeks (A) and average speed on horizontal ladder (C) is significantly lower at 15 weeks in COX10Adv mutant mice compared with controls; F-G: The average weight of COX10Adv mutant mice is not significantly different compared with control mice at 13 weeks following completion of tamoxifen gavages, time point at which focal spinal cord demyelination was carried out. The survival of COX10Adv mutant mice, based on the development of a moderate clinical phenotype according to UK Home Office guidelines, is not compromised until 17 weeks following completion of tamoxifen gavaging.

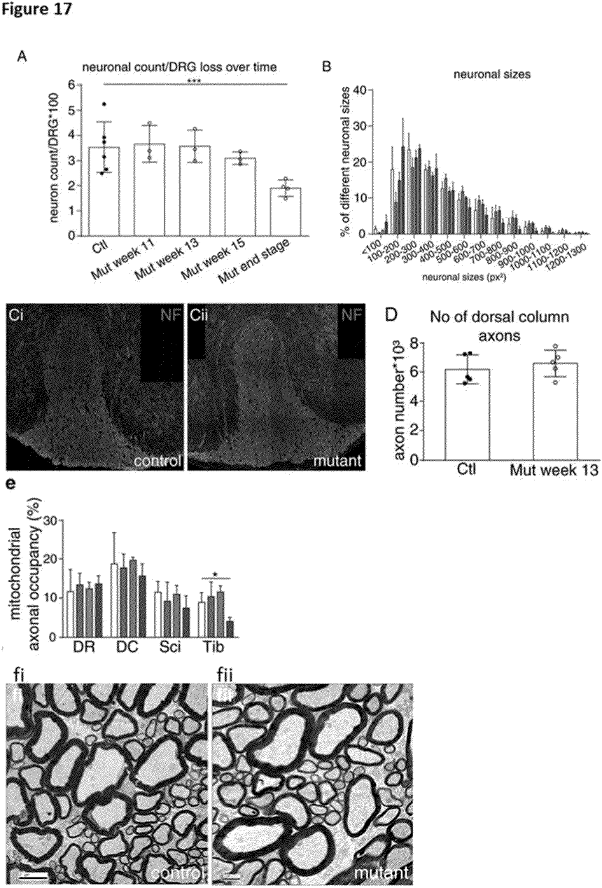

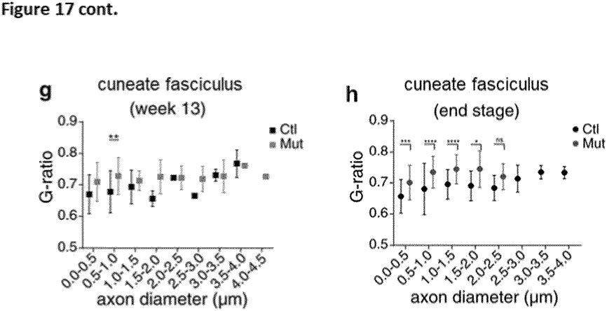

FIG. 17 illustrates no evidence of neurodegeneration at the time point when experimental demyelination is carried out wherein A-B: The total number of neurons in the lumbar DRG, when counted in every 5 serial sections of the DRG, was not significantly different in COX10Adv mutant mice compared with control mice, at 13 weeks following the completion of tamoxifen gavages when experimental demyelination is carried out (A). Furthermore, the average area of DRG neurons in cresyl violet staining indicates the preservation of relatively large neurons at the time point when demyelination is carried out (B); C-D: In cross-sections of the thoracic spinal cord, the total number of dorsal column axons were not significantly different in COX10Adv mutant mice (C and Dii) compared with control mice (C and Di); E-F: The average g-ratio of dorsal column axons in COX10Adv mutant mice was not significantly different from control mice at 13 weeks following the completion of tamoxifen gavages when experimental demyelination is carried out (E, left). In contrast, the average g-ratios are significantly greater in COX10Adv mutant mice at the end stage compared with control mice, indicating potentially thinner myelin (E, right). Degenerating axons were absent in both COX10Adv mutant mice (Fii) and control mice (Fi) at 13 weeks following the completion of tamoxifen gavages when experimental demyelination is carried out; G: Mitochondrial content of myelinated axons, as a percentage of the area of the axon occupied by mitochondria, was not significantly different at the time point when experimental demyelination was carried out. In contrast, a significant depletion of mitochondria was apparent within myelinated axons in sciatic nerve and tibial nerve at the end stage of COX10Adv mutant mice compared with control mice, as expected.

FIG. 18 illustrates modeling the complex IV deficiency in DRG neurons, in vitro wherein A-C: Application of sodium azide [SA (100 µM for 16 hours)] to DRG neurons, in vitro, significantly decreased mitochondrial respiration in SeaHorse analysis (A-B), as expected, without compromising cell viability (not shown). Mitochondrial respiration decreases when DRG neurons that are over expressing Miro1, over expressing PGC1a or treated with pioglitazone are exposed to SA (100 µM for 16 hours) (C).

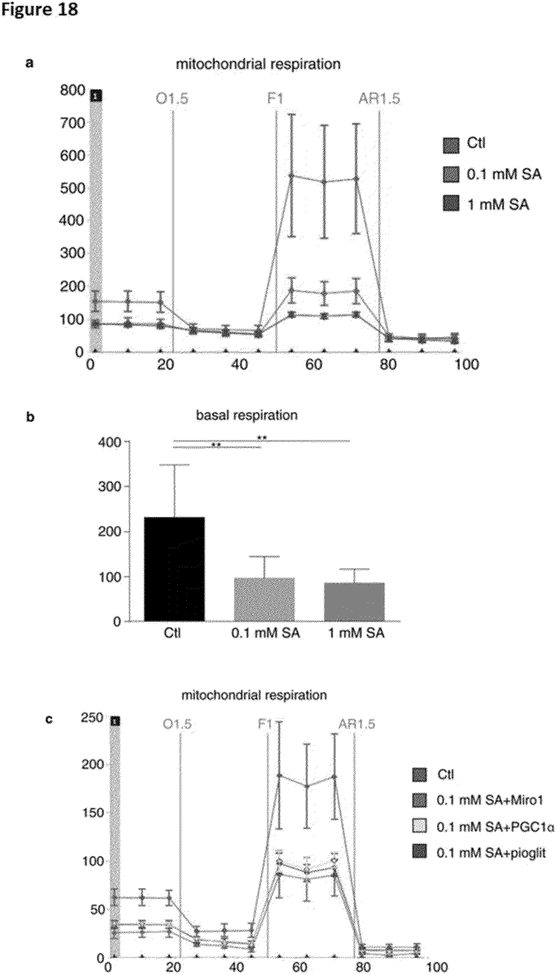

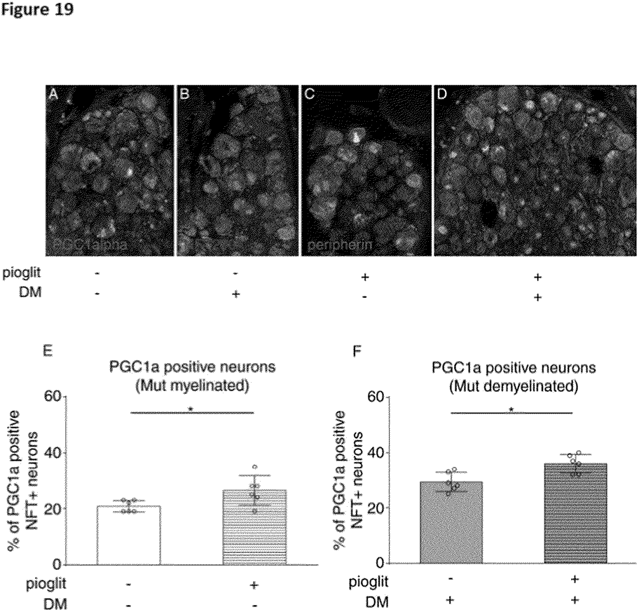

FIG. 19 illustrates PGC1a positive nuclei within DRG neurons in COX10Adv mutant mice increases significantly following focal demyelination of the dorsal columns and administration of pioglitazone in diet wherein A-F: PGC1-alpha positive neuronal nuclei (green) are relatively infrequent within DRG neurons from COX10Adv mutant mice on chow diet (A, untreated and not demyelinated). Pioglitazone in diet for 6 weeks significantly increased the percentage of DRG neurons with PGC1-alpha positive nuclei (C and E) in COX10Adv mutant mice (neg) compared with COX10Adv mutant mice on chow diet (A and E). Demyelination of the dorsal columns of untreated COX10Adv mutant mice (neg), using lysolecithin, increased in PGC1-alpha positive DRG neuronal nuclei (B) compared with untreated and non-demyelinated COX10Adv mutant mice (A, E and F). Demyelination of the dorsal columns of pioglitazone treated COX10Adv mutant mice (neg), using lysolecithin, further increased in PGC1-alpha positive DRG neuronal nuclei (D and F) compared with untreated and demyelinated COX10Adv mutant mice (B and F).

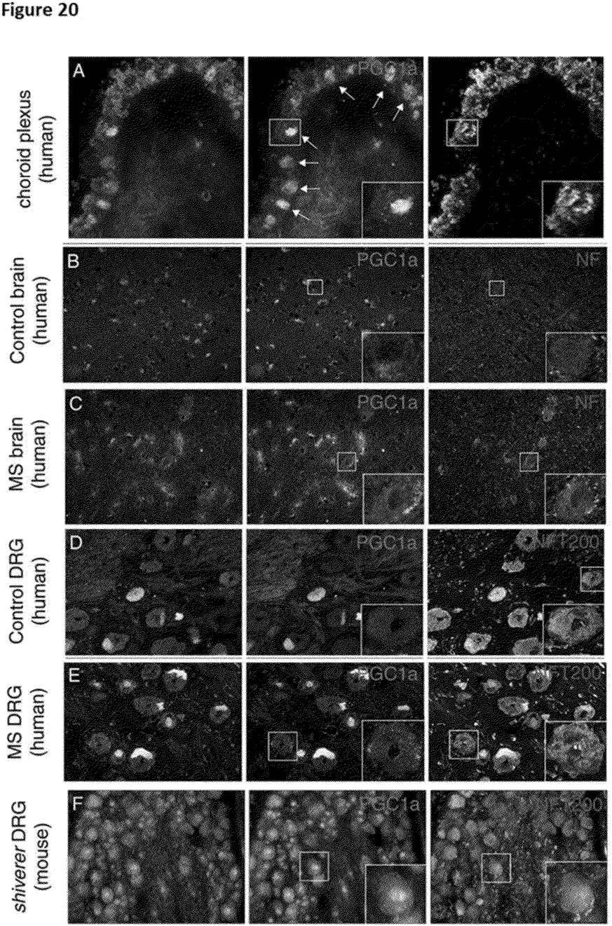

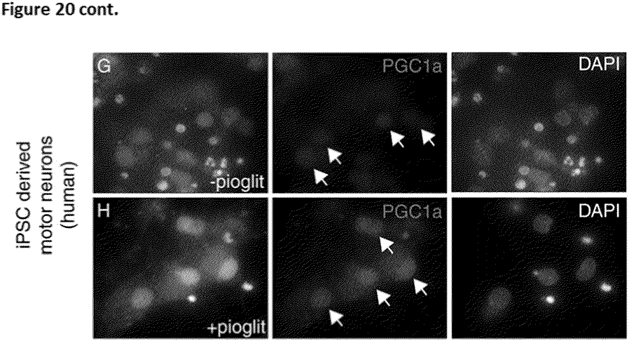

FIG. 20 illustrates validation of PGC1a as a target to protect demyelinated axons in MS wherein A: PGC1a-positive nuclei (green, arrows) are evident within choroid plexus epithelial cells in multiple sclerosis, which contains complex IV deficient cells [stained blue (complex ll 70 kDa subunit) and lacking red (complex IV subunit-l)]; B-E: PGC1a-positive nuclei are not detected within neurons using immunofluorescent labeling within the cerebral cortex (B-C) and DRG neurons (D-E) in MS (C and E) and controls (B and D); F: The lack of PGC1a nuclei in neurons in MS tissue is unlikely to be because demyelination is chronic, as opposed to the experimental demyelination being acute, as PGC1a positive nuclei are evident within DRG neurons in Shiverer mice with dysmyelinated axons, which models chronic demyelination; G-H: Human iPSC derived motor neurons in culture showed faintly PGC1a-positive nuclei (G, arrows). Following exposure to pioglitazone (2microM) human iPSC derived motor neuronal nuclei showed intense labeling of PGC1a (H, arrows), indicating that pioglitazone can target PGC1a in human neurons.

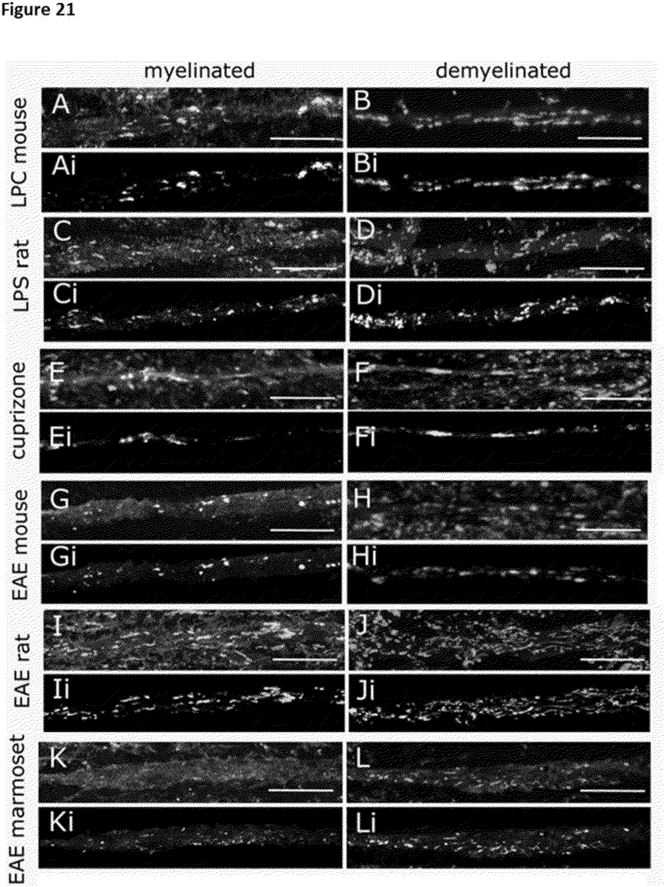

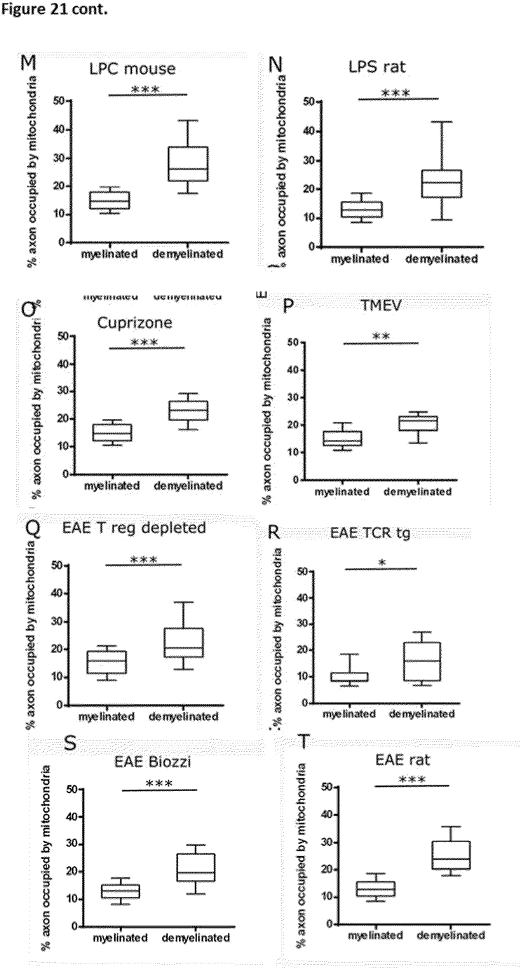

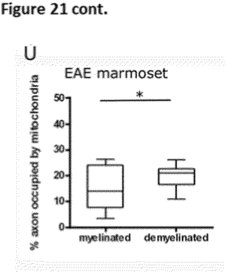

FIG. 21 illustrates increased axonal mitochondria in a range of experimental demyelination. Compared with myelinated axons from controls, in triple labelled immunofluorescent confocal images (column to the left with MBP in red, neurofilament-H in blue and porin in green), mitochondria are more prevalent in demyelinated axons (column to the right) from all the models. The grey scale images (21Ai-Li) show porin-positive elements within axons from the corresponding triple labelled colour images (21A-L). The quantitation of axonal mitochondrial content shows a significant increase in the lysolecithin-induced focal lesions (LPC, 21A-B and 21 M), lipopolysaccharide-induced focal lesions (LPS, 21C-D and 21N), cuprizone model (21 E-F and 210), Theiler’s murine encephalomyelitis virus (TMEV) model (21 P) as well as experimental autoimmune encephalitis (EAE, 21G-L and 21Q-U) in mice (C57BL6, SJL/J and Biozzi ABH), rat (Dark agouti) and marmoset species (the area of porin-positive elements as a percentage of axon area). 20 axons per region were randomly selected from each animal for quantitation. The box plots indicate the median, inter-quartile range (25%-75%) and 90% confidence interval. *p<0.05, **p<0.01 and ***p<0.01 . Scale bar indicates 10 µm.

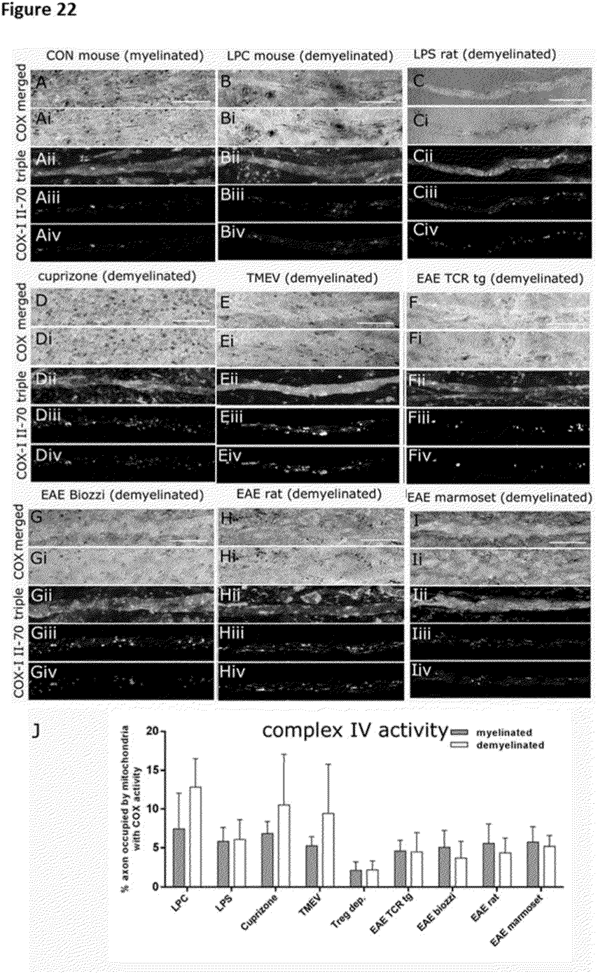

FIG. 22 illustrates complex IV activity within axons and the detection of complex IV subunit-l relative to complex ll 70 kDa in complex IV-deficient axonal mitochondria. Complex IV activity can be localized to the axon by the sequential complex IV histochemistry (bright field images, 22Ai-li) and triple immunofluorescent labelling (22Aii-lii) of neurofilament (green), complex ll 70 kDa subunit (red) and complex IV subunit-l (blue) and then by merging the bright field image with triple labelled immunofluorescent image (22A-I). This sequential technique immunofluorescently labels the complex IV-deficient mitochondria (labelled with complex II 70 kDa, 22Aiii-liii) and their mitochondrial respiratory chain complex subunits (22Aiv-liv), as previously described (82). The grey scale immunofluorescent images of axonal complex II 70 kDa (Aiii-liii) and complex IV subunit-I (22Aiv-liv) are generated by splitting the corresponding triple labelled colour image (22Aii-lii) and clearing the non-axonal mitochondria. As reported previously, the mitochondria with complex IV activity, evident in the bright field images, are not immunofluorescently labelled (82). Following lysolecithin-induced (LPC) demyelination (panel B), there are numerous axonal mitochondria with complex IV activity and elongated morphology (22B and 22Bi) compared with myelinated axons from controls (22A and 22Ai). In contrast, mitochondria with complex IV activity in demyelinated axons are less numerous and rounded or less elongated in all other models: lipopolysaccharide-induced (LPS) lesions (22C-Ci), cuprizone model (22D-Di), TMEV model and experimental autoimmune encephalitis (EAE) in mouse, rat and marmoset. The quantitation of complex IV activity within axons shows a significant increase following LPC-induced focal demyelination (22J). 20 axons per region were randomly selected from each animal for quantitation. The bar charts indicate the mean plus standard deviation. *p<0.001. Scale bar indicates 10 µm.

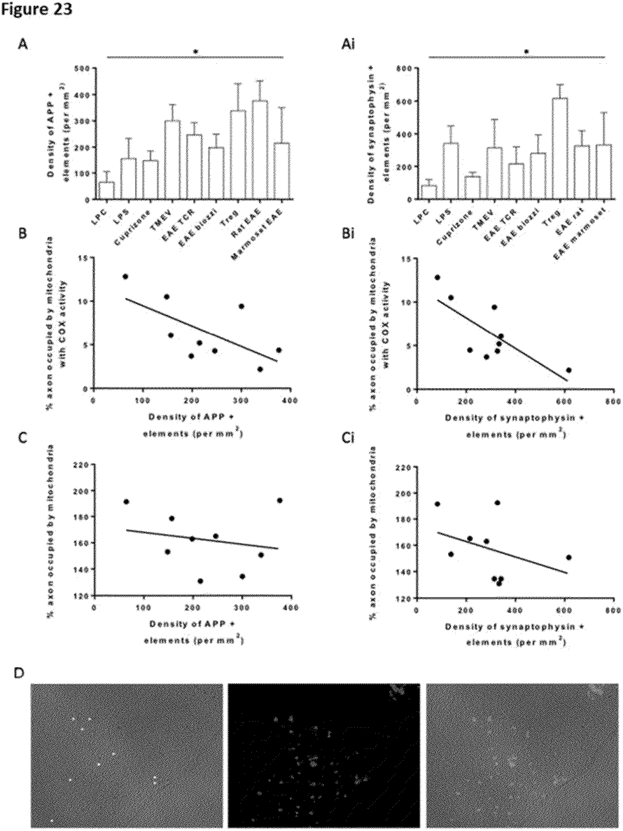

FIG. 23 illustrates the association between complex IV activity within demyelinated axons and extent of axonal injury. The density of axonal injury, judged by amyloid precursor protein (APP, 23A) and synaptophysin (23Ai) labelling, varies considerably between the disease models (ANOVA p<0.001). There is a significant inverse correlation between complex IV activity within demyelinated axons and axon degeneration, judged by the density of APP (23B, r2 = 0.421, p=0.048) as well as synaptophysin (23Bi, r2 =0.561, p=0.020) labelling. In contrast, a significant correlation is not found between mitochondrial content in demyelinated axons and the density of APP (23C, r2 =0.040, p=0.604) and synaptophysin (23Ci, r2 =0.147, p=0.308) labelling. A sequential COX histochemistry and immunofluorescent labelling of APP shows that a subset of APP and synaptophysin labelled structures contains mitochondria with complex IV activity in all nine disease models (23D, synaptophysin positive structures lacking complex IV activity are shown in T-reg depleted EAE lesion, arrowheads). The bar charts indicate the mean plus standard deviation.

FIG. 24 is a schematic representation of the difference in macrophage response between complex IV deficient and complex IV efficient demyelinated neurons.

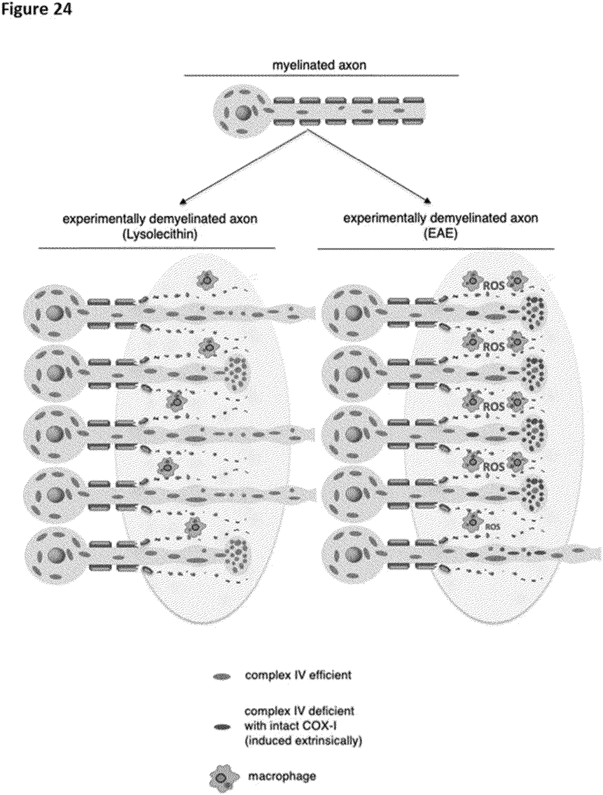

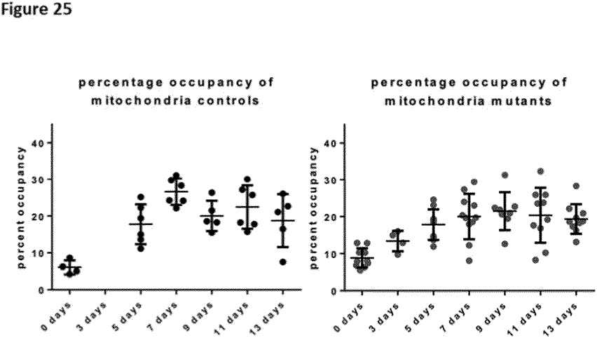

FIG. 25 illustrates the time course of ARMD in sciatic nerves. The graphs demonstrate changes over time in axonal mitochondrial content following experimental focal demyelination of the mouse sciatic nerve (wild type mice on the left and complex IV mutant mice on the right), reflecting axonal response of mitochondrial to demyelination (ARMD). ARMD peaks at day 7 and 9 in wild type mice and complex IV mutant mice, respectively. ANOVA p <0.0001 for both groups.

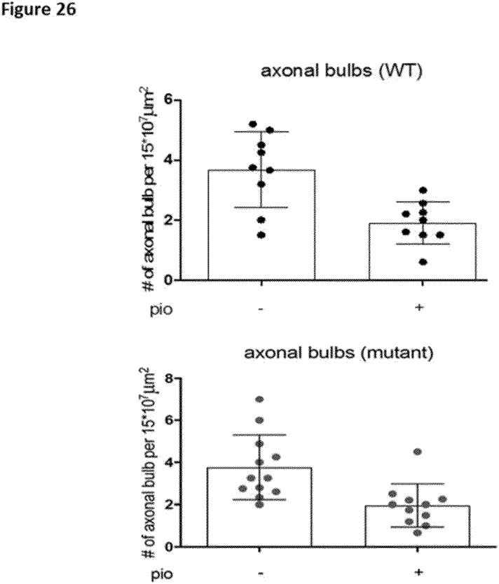

FIG. 26 illustrates that the enhancement of ARMD by pioglitazone protects demyelinated sciatic nerve axons. Pioglitazone in diet led to a significant decrease in the extent of axonal injury (transections indicated by axonal bulbs) in focal demyelinated sciatic nerve lesions in both wild type mice (left, p= 0.0083) and complex IV mutant mice (right, p=0.0007), at 8 days post focal lesioning.

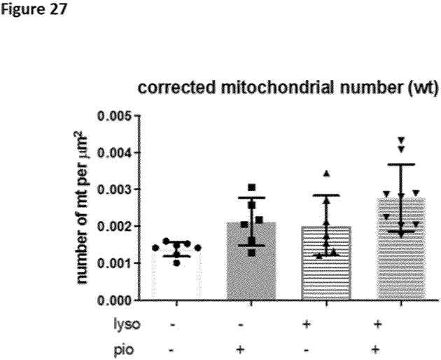

FIG. 27 illustrates focal demyelination (lyso+ pio-) caused significantly increased axonal mitochondrial number, compared with non-demyelinated axons (lyso- pio-), reflecting ARMD response. Pioglitazone treatment significantly increased axonal mitochondrial content in non-demyelinated axons and tended to increase axonal mitochondrial content in demyelinated axons at 8 days post focal lesioning.

### DETAILED DISCUSSION OF THE INVENTION

The inventors propose a mechanism (FIG. 7) based on their determinations that demyelination per se creates a relative shortfall in the energy producing capacity, through the inability of the axon to rapidly increase its mitochondrial content; thus, the axon is not able to meet the increased energy demand that follows the loss of myelin. Myelination is associated with a decrease in axonal mitochondrial content, as evident in myelinated optic nerve axons and unmyelinated axonal segments in lamina cribrosa as well as demyelinated axons in Shiverer mice (FIGS. 7a-b and e) [3, 5]. Upon demyelination, the inventors determined that mitochondria mobilise from neuronal cell body to the acutely demyelinated axons, and slowly building up its mitochondrial content, through ARMD (FIGS. 7b-c and f). Although the neuronal cell body attempts to energetically support the demyelinated axon, by increasing the transport of mitochondria to the axon, the peak of ARMD lags behind the peak of axon transection by a number of days. Thus, the homeostatic response of ARMD is insufficient to correct the resulting energy imbalance created by demyelination (FIG. 7f). By targeting mitochondrial biogenesis and anterograde transport to enhance ARMD, the inventors have identified a novel therapeutic strategy to protect the acutely demyelinated axons (FIGS. 7c-d and g).

Currently, there is no effective neuroprotective therapy for demyelinating disorders, including MS. Whilst an existing strategy has sought to curtail the increased energy demand of demyelinated axon through drugs that inhibit sodium channels, although effective in experimental models, this has failed in clinical trials and is poorly tolerated by MS patients [27, 76]. This is perhaps not surprising because they perturb adaptive neuronal firing properties that are required for healthy functioning. An alternative strategy, shown herein, is to boost the energy producing capacity of the demyelinated axon. Whilst previous studies have shown that axonal mitochondrial transport can be increased by targeting mitochondrial biogenesis, through PGC1α over expression, as a potential therapy for neurodegenerative disorders [50], this potential therapeutic strategy, has not been applied to demyelinating disorders. The inventors have shown that increased transport of mitochondria from the neuronal cell body to the axon makes ARMD more efficient and protects the acutely demyelinated axon. Given the suboptimal nature of the homeostatic ARMD, the strategy of increasing mitochondrial biogenesis and axonal transport is ideally suited for demyelinating disorders. Therefore provided herein is a mechanism that can be therapeutically targeted — within the necessary short time frame — for neuroprotection in demyelinating disorders.

The inventors undertook detailed analysis of mitochondria in DRG neuronal cell bodies from 18 progressive MS autopsy cases and 12 controls. Furthermore, they correlated mitochondrial changes within DRG neuronal cell bodies with mitochondrial changes within demyelinated axons, at the dorsal root entry zone, in spinal cord blocks. Complex IV deficiency and clonally expanded mtDNA deletions in DRG neurons, as observed in this study, were similar to those seen in cortical neurons and choroid plexus epithelial cells in MS, although the respiratory deficiency affected a greater proportion of neurons in the DRG [7, 9]. Factors other than the energy shortfall due to the time lag of ARMD and inflammation induced damage to mitochondria are present which are intrinsic to the neuron that contribute to the energy deficit of the demyelinated axon. MtDNA deletion in single cells is not a reflection of the accumulation of ongoing damage to mtDNA. Clonal expansion of mtDNA is an active phenomenon that amplifies a mtDNA deletion in a single cell, as evident in a number of neurodegenerative disorders, including MS [29]. In MS, mtDNA deletions appear to be induced by the inflammatory process. These mtDNA deletions then undergo amplification through clonal expansion in metabolically highly active cells such as neurons and choroid plexus epithelial cells [8]. Given the clonally expanded mtDNA deletions in DRG neurons, together with the significant positive correlation between the extent of complex IV deficiency in proprioceptive DRG neuronal cell bodies and axonal mitochondrial content, the inventors suggest that complex IV deficient neurons attempt to trigger ARMD even more vigorously than neurons with healthy mitochondria.

The failure to identify respiratory deficient neurons in the brain, spinal cord and DRG following a detailed examination in nine experimental disease models, and tissue from spinal cord hemi-section, highlights a major short fall of the existing disease models in recapitulating neuropathological findings of progressive MS. There are multiple potential reasons for the lack of respiratory deficient neurons in existing models. First, oxidative injury that is implicated in the induction of mtDNA deletions is limited in established disease models compared with MS [60]. Second, these experimental disease models predominantly adopt young animals, which may have better repair and mitochondrial quality control mechanisms. Third, the clinicopathological course of animal models is relatively short compared with the disease duration of progressive MS. Finally, both chronic and ongoing acute demyelination, as evident in slowly expanding MS lesions, are relatively sparse in existing disease models compared with progressive MS [19]. The complex IV knockout mice used in this study help to determine the consequences of mitochondrial respiratory chain deficiency for the demyelinated axons. The inventor’s data in COX10Adv mutants indicate that the stimulation of mitochondrial biogenesis and mitochondrial dynamics can partly overcome the detrimental consequences of the complex IV deficiency in acutely demyelinated axons. The protective effect of increasing mitochondrial biogenesis in complex IV deficient neurons is likely to reflect the fact that such mitochondria are deficient, but not completely devoid of metabolic activity. Therefore, despite their deficiency, they exert a net positive effect, and help boost the overall energy producing capacity of the axon. The inventors therefore suggest that enhancing ARMD is a therapeutically tractable approach, particularly when combined with approved therapy in MS as well as HIV neuropathy and diabetic neuropathy, where complex IV deficiency is an additional contributor to the axonal energy failure [6, 9, 14, 78].

In demyelinating disorders, axon degeneration is most prominent in areas with acute demyelination. In MS, neuropathological studies have shown that demyelination is ongoing throughout the clinical course of the disease. During the early stage of MS, there is an abundant formation of acutely demyelinating lesions [19]. In progressive MS, there is still ongoing demyelination, particularly at the edge of chronic active or slowly expanding lesions even at the end stages of MS. [19, 35] MRI imaging provides robust evidence of new lesions in early stage and slowly expanding lesions [1, 11, 19]. In terms of neuronal mitochondria, factors that amplify mitochondrial injury, such as oxidative stress and iron accumulation, likely require time to compromise the neurons [37]. The inventors consider that respiratory deficiency within neurons becomes more prominent with increasing disease duration in MS. Thus, the inventors consider their findings in both wild type mice and COX10Adv mutant mice show that the neuroprotective strategy discussed herein is applicable to the entire disease course of MS.

In summary, the inventors have determined that where mitochondria within neuronal cell bodies respond to the increased energy demands of demyelinated axons by moving to the axon (ARMD):
Homeostatic ARMD is insufficient to protect all acutely demyelinated axons.
ARMD can be enhanced by (i) increasing the movement of mitochondria from the neuronal cell body to the axon and (ii) mitochondrial biogenesis in the neuron.
Enhancing ARMD (genetically or pharmacologically) protects acutely demyelinated axons - in multiple experimental systems (incl. microfluidic chambers, where the neuronal cell body is specifically targeted, brain slices and in vivo models) - the acutely demyelinated axon of both wild type neurons with healthy mitochondria as well as those neurons in disease states that harbour complex IV deficiency.
Increased mitochondrial content in autopsy derived axons from progressive MS cases, where cytochrome c oxidase (complex IV) deficient neurons are evident, suggests that even the complex IV deficient neurons attempt to support the energetics of demyelinated axons.
Rescue of demyelinated axons by enhancing ARMD in complex IV deficient neurons, shows a protective effect in disease states despite the complex IV deficiency.

The neuroprotective strategy proposed by the inventors is to protect acutely demyelinated axons from damage. For example, in progressive MS rather than relapsing disease, wherein neuropathological studies and MRI measurements show that there is ongoing demyelination in progressive MS, particularly at the edges of chronic active lesions which are slowly expanding even with optimal disease modifying therapy and at the later stage of progressive MS (at autopsy), this strategy provides an advantageous treatment option which was not considered as demyelination was previously not considered not to correlate well with disability in progressive MS.

### EXAMPLES

### Methods

### Preparation of Cerebellar Slice Culture

Cerebellar slices were prepared as previously described [4]. Briefly, wild type C57BL/6 and Shiverer mice pups were sacrificed at P10 and cerebellum was placed in ice-cold dissection medium. The sagittal slices were then sectioned into 300 µm thick slices and placed on a membrane insert. Picospritzer III (Parker, US) and a micromanipulator was used to inject mEOS2-Lentivirus (titre 7-8*10⁹; aliquots stored at -80° C.) containing 0.025% of Fast-Green (FG, Sigma F7258, UK) in to the Purkinje cell body layer. To enhance activation of the CMV-promoter driven mEOS2 construct, 10 µM of Forskolin (Forskolin Coleus forskohlii, 344282 Sigma) was added to the slice culture medium the day after injection and removed 2 days later. To demyelinate cerebellar slices, the inventors used lysolecithin (L-α-Lysophosphatidylcholine; L4129 Sigma), which minimally impacts mitochondrial function [4]. 0.75 mg/ml Lysolecithin was added to the medium for 17 hours at DIV13. For the time course experiments of the ARMD, 0.5 mg/ml lysolecithin was added to the medium for 17 hours. The slices were then fixed and stained using immunofluorescence histochemistry at several timepoints after removal of lysolecithin to determine changes in axonal mitochondrial parameters and axonal bulbs. Only demyelinated axons that were not transected were chosen for axonal mitochondrial analysis.

### Preparation of Viral Particles

Cloning and preparation of Lentivirus was performed as previously described [41]. Miro1 and PGC1α plasmids were sourced from Addgene (pRK5-Miro1, plasmid #47888; AAV-CMV-Flag-PGC1α-6His, plasmid #67637). Miro1 was inserted into a pDONR-P2A-mKate2 vector. The pDONR-P2A-mKate2 vector was used as a negative control and as an axonal marker in live imaging experiments. PGC1α was inserted into a pDONR-P2A-eGFP. The m1 m4-eGFP (kind gift from Alan Peterson and Anna Williams) was amplified using primers with attB sites and cloned into a pDONR vector. All plasmids were shuttled into a lentiviral backbone pLenti6-cppt-delta CMV-DEST-opre, as described previously¹¹. The viral titers were as follows mEOS2 8.2*10⁹ cfu/ml, Miro1 5*10⁸ cfu/ml, PGC1α 1.4*10⁸ cfu/ml, mKate2 2.25*10⁹ cfu/ml, m1m4-eGFP 6.74*10⁹ cfu/ml.

### Live Imaging and Analysis of Axonal Mitochondria in Cerebellar Slices and Identification of Demyelinated Axons In-Vitro Using Spectral Confocal Reflectance Microscopy (SCoRe)

Live imaging of mitochondria in Purkinje cell axon was performed at DIV14, directly after removing the lysolecithin. Mitochondria in the most proximal 50 µm segment of the Purkinje cell axon were photoconverted using the 405 nm laser at 3% laser power for 20 seconds [34]. Immediately after photoconversion, the 85 µm long proximal axonal segment was imaged every minute for 20 minutes. An 8-12-micron stack was created from images every 0.5 micron in depth. Time lapse images were used to generate videos, exported from the Zeiss software, while all further analysis was done in Fiji [59]. Newly transported mitochondria, either appearing from the neuronal cell body to the most proximal 20 micron axonal segment or distal axonal mitochondria appearing in the 20 microns long axonal segment of the most distal photoconverted segment, were counted visually and confirmed using the kymograph. Green mitochondria represent newly transported mitochondria and red labelled mitochondria represent pre-existing mitochondria in the proximal axonal segment. For each mitochondrion appearing, the direction of movement was noted, as well as the area of the mitochondrion was measured manually, using the measure function in Fiji, when it first appeared in the axon.

Kymographs of the time lapse images were generated by using the ImageJ plugin KymographClear2.0 [40]. Mitochondrial speed of movement was determined by using the Kymotoolbox ImageJ plugin [80]. The mitochondria moving anterograde from the cell body to the axon were identified by the slope direction of tracks within the 20 micrometers of the kymograph and subsequently confirmed on the video. For retrograde moving mitochondria 20 micrometers of the most distal part was used.

The inventors used spectral confocal reflectance microscopy (SCoRe) to determine the myelination status of the axons [58]. Specificity of SCoRe, to differentiate demyelinated axons from myelinated axons, was confirmed by immunofluorescence staining for Myelin Basic Protein (MBP) and neurofilament and then overlapping it with SCoRe image.

### Triple Immunofluorescent Staining and Confocal Imaging of Cerebellar Slices

The membrane inserts containing cerebellar slices were cut out and fixed in PFA before heat mediated antigen retrieval. After blocking with Normal goat serum (NGS, Vector S-1000, US), the free floating slices were incubated with three primary antibodies (Table 3), and then exposed to secondary antibodies, before mounting using Vectashield with DAPI (Vector H-1200, US) [52]. Confocal images of triple staining were acquired on a Zeiss LSM 710 inverted confocal microscope (Zeiss, Germany). To quantitate axonal mitochondria, images were processed in Adobe Photoshop CS6 (Adobe, US) and all non-axonal mitochondria (outside the NF+ structures) were removed. Images were then opened in Fiji and individual axons were cut out to analyse the axonal mitochondrial parameters using macros. The first macro split the images into the separate colour channels, the second macro measured the axonal area and the third macro measured the axonal mitochondrial number and area. For each slice, 40 axons were chosen and the mean axonal mitochondrial occupancy was calculated for each data point, which represents a slice from a different animal. For the analysis of axonal bulbs, images of NF labelling were acquired as mentioned above and the axonal bulbs per field of view in x63 images were counted. For each cerebellar slice 5 fields of view were randomly selected and the axonal bulb were counted and averaged for each datapoint.

### Triple Immunofluorescent Staining and Confocal Imaging of Mouse Spinal Cord and MS Cryosections

Longitudinal cryosections, 15 micrometer thickness, of dorsal spinal cord were placed on glass slides and stored at -80° C. The triple immunofluorescent staining and confocal images were taken from wild type and mutant mice as well as human tissue and followed the same protocol as for the cerebellar slices.

For the analysis of axonal mitochondria, only the non-transected dorsal column axons were included. Acutely demyelinated experimental lesions were identified by DAPI staining and loss of MBP staining. Chronic MS lesions were identified by loss of MBP and serial sections were used for the mitochondrial analysis. Images were processed and analysed as described for cerebellar slices. For calculating mitochondrial complex IV deficiency in mouse spinal cord axons (wild type and mutant) and human dorsal column axons, the mitochondrial channels of complex II 70 kDa and COX-l were merged in Fiji. A macro was used to calculate the percentage of complex ll 70 KDa-positive regions that were co-labelled by COX-l for each axon. For each case, 40 axons were chosen and the mean axonal mitochondrial occupancy was calculated for each data point, which represents a different animal or human case. Axonal bulbs were quantitated as described for cerebellar slices.

For the analysis of axon number in dorsal columns of WT and COX10Adv mutant mice, frozen transverse sections of cervical spinal cord were fixed and stained, as described previously. The sections were then imaged on a Zeiss ApoTome.2 (Zeiss, Germany) with tile scan function and stitching to generate a single image file for the whole dorsal column. The total axon number per animal was determined using “analyse particle” function in FIJI. For the analysis of PGC1α-positive nuclei in DRG neurons, snap frozen spinal cord was cryosectioned longitudinally, fixed and stained for PGC1α, NF200 and peripherin. Images of the whole DRG were acquired and the percentage of PGC1α+ NF200+ cells was determined. For the analysis of mitochondrial respiratory chain subunits of the mitochondrial respiratory chain within human DRG neurons, the inventors triple stained using NF200, complex ll 70 kDa and a number of subunits. The percentage of DRG neurons that were deficient in mitochondrial respiratory chain subunits was determined in serial sections of human DRG.

### Design and Fabrication of Microfluidic Chambers

Microfluidic chambers were fabricated in polydimethylsiloxane (PDMS) (Sylgard 184, Dow Corning, US) using standard soft lithography techniques, comprising of an array of microchannels between two culture chambers that are fluidically addressable via inlet/outlet wells, as previously described [36]. Prior to use, these devices were coated with 0.45 mg/ml Matrigel (Corning 356231, US) for 1 hour at RT and 30 mg/ml Poly-D-lysine (PDL, Sigma Aldrich P6407, UK) for 30 minutes at RT. To achieve fluidic isolation in a given chamber the inventors added at least 10µl more of the medium to the opposite chamber, which prevents diffusion from the treated to the untreated chamber, as previously shown [55, 56].

### Culture of DRG Neurons and OPCs in Microfluidic Chambers

DRG neurons were rapidly extracted from P4-P8 old C57BL/6 mice pups, as previously described [64]. Differential adhesion was used to remove excess glial cells from the culture. After seeding the DRGs in seeding medium a concentration of 20 µM FUDR was added to the DRG neuronal cell body chamber and the axonal chamber to reduce growth of non-neuronal cells. A concentration gradient of 25 ng/ml NGF in the cell body chamber to 50 ng/ml NGF in axonal side was created to enhance axonal growth. The day after placing DRG neurons in the cell body chamber, the seeding medium was completely removed and replaced with maintenance medium. Until the 10 day after seeding, FUDR was added at a concentration of 10 µM to both sides of the chamber, while the NGF gradient was maintained for the same period. At 10 days after seeding Lentivirus expressing mKate2 was added to the cell body chamber at MOl 25 to label DRG neurons in the cell body chamber and their axons in the axonal chamber.

OPCs for myelinating cultures were obtained by dissection of Sprague Dawley rat cortices, as previously described [82]. Once cell count was done using a hemocytometer, M1-M4-eGFP lentivirus was added at MOl 40 to approximately 100,000 OPC. Oligodendrocyte precursor cells (OPCs) were seeded into the axonal chamber at 12 days following DRG seeding in the cell body chamber, in order to allow sufficient number of axons to have crossed into the cell body chamber [72]. Immediately following seeding of OPCs, the maintenance medium in both chambers was replaced with myelination medium [72]. Thereafter co-cultures were maintained for another 2 weeks, while renewing the myelination medium 2 times a week, to ensure adequate myelination prior to the visualization of myelinated axonal segments in live-imaging.

### Demyelination of Microfluidic Chambers and Analysis of Axonal Damage and Myelin-Status in Live Images

The inventors established that 0.005 mg/ml of lysolecithin for 2 hours was sufficient to demyelinate DRG-OPC co-cultures without damaging the DRG axons in unmyelinated cultures. Before adding lysolecithin to microfluidic chambers, the inventors used an Axio Observer Z1 inverted motorized microscope (Zeiss, Germany), for live imaging to identify the myelinated axonal segments (video 11, online resource). Images of the myelinated axonal segments were saved with x-y coordinates of the stage position. The effect of live imaging on the axons and myelin was determined by imaging microfluidic chamber containing both mKate2 expressing DRGs and eGFP-expressing OPC where imaged at 20x magnification (Plan-Apochromat 0.8 NA Ph 2 M27 objective, Zeiss, Germany) for 30 minutes before returning the microfluidic chambers to the incubator. Re-imaging 24 hours later showed that there was no significant effect of live imaging on axonal health.

The inventors then added lysolecithin for 2 hours before live imaging the previously imaged axonal segments again, using the x-y co-ordinate of the stage position. These live images of axons, pre and post lysolecithin, allowed the assessment of the demyelination of the axonal segments (based on M1-M4-eGFP fluorescence) as well as axonal damage following acute demyelination (based on appearance of mKate2 fluorescence). Axonal structure was categorized as intact, beaded or fragmented, both prior to and following exposure to lysolecithin. Intact axons showed continuous mKate2 fluorescence and beaded axons showed obvious irregularities in axon diameter without transection. Meanwhile, axons were classed as fragmented when the mKate2 fluorescence was disrupted and not continuous in at least one part of the axon. Nearly all myelinated axonal segments were intact prior to exposure to lysolecithin and only intact myelinated axons were considered for the assessment of axon damage following acute demyelination. Following exposure to lysolecithin, demyelination was confirmed based on disruption or loss of M1-M4-eGFP fluorescence. On average 12 myelinated axonal segments were included per microfluidic chamber. An average of data from 2-3 microfluidic chambers per batch of experiments were pooled to generate a single data point presented in FIG. 3.

### Manipulating Mitochondrial Dynamics in Unmyelinated and Myelinated Cultures

The inventors targeted anterograde movement of mitochondria in unmyelinated DRG neurons, by over expression Mitochondrial Rho GTPase1 (Miro1), using a lentivirus [22]. Furthermore, mitochondrial biogenesis in neurons was targeted by over expressing peroxisome proliferator-activated receptor gamma (PPAR-y) coactivator 1-alpha (PGC1α), using a lentivirus [25, 54]. The inventors then pharmacologically targeted mitochondrial biogenesis in neurons by using pioglitazone [44]. DRG were extracted as described previously and seeded on glass-bottomed 35 mm dish (µ-dish35mm, low Grid-500 ibiTreat, ibidi 80156, Germany). Lentivirus Miro1 at MOl 10 was added to the culture medium at seeding (3 weeks before live imaging) and Lentivirus PGC1α at MOl 10 was added at 2 and 4 days and 3 weeks before live imaging. Finally, 2 µM pioglitazone was added at 2, 4 and 6 days and 3 weeks before live imaging and renewed with each media change. The same manipulations were carried out in DRG neurons co-cultured with OPCs, which were added DIV12. Myelinated axonal segments were identified for confocal imaging using SCoRe, as previously described for cerebellar slices (FIG. 8).

### Manipulating Axonal Mitochondria by Targeting DRG Neuronal Cell Bodies in Microfluidic Chambers

In order to avoid pioglitazone impacting oligodendrocyte lineage cells and myelinated axons, the drug was applied to the DRG neuronal cell body chamber at a concentration of 2 µM for 6 days prior to demyelination. Pioglitazone was renewed in the neuronal cell body chamber with each media change. Furthermore, PGC1α inhibitor [15 µM SR-18292 (SML2146, Sigma UK)] was added together with pioglitazone to neuronal cell body chamber [62]. Following 6 days of pioglitazone treatment of the neuronal cell bodies, with or without PGC1α inhibitor.

Following 6 days of pioglitazone treatment of the neuronal cell bodies the inventors added lysolecithin to the axonal chamber to demyelinate, as previously described.

Lentivirus Miro1 and Lentivirus PGC1α were applied to the neuronal cell body chamber at seeding and DIV16, respectively. At DIV26, lysolecithin was added to the myelinating chamber, as previously described. Axonal integrity of myelinated segments (before demyelination) and axonal damage following demyelination (of the same myelinated axonal segment) were quantified using live imaging, as previously described.

### Manipulating Axonal Mitochondria in Cerebellar Slices

The cerebellar slices from wild type C57BL/6 mice pups (P10) were prepared as described and the slices were maintained on membrane inserts in culture for a week, before adding 40 µM pioglitazone (Sigma PHR1632, UK) to the culture medium. Two days following exposure to pioglitazone, culture medium was renewed and lysolecithin 0.5 mg/ml was added to the culture medium for 17 hours. Following the removal of lysolecithin, pioglitazone was replaced in the culture medium for 3 days until fixing and staining of the slices, as previously described. Triple immunofluorescent labelling and confocal microscopy were used to assess demyelination and axonal bulb formation as well as mitochondrial occupancy of non-transected and demyelinated axons, as previously described.

### Photoconversion of mEOS2 and Live Imaging of Axonal Mitochondria in DRG Neurons

Live imaging of mitochondria located in the proximal segment of DRG axon, was performed with or without mitochondrial manipulations, as described above for cerebellar slices. Each data point on the graphs in FIG. 2 represents the value of a single axon.

To assess the ARMD response, DRG neurons were seeded in microfluidic chambers and mEOS2 lentivirus was added at seeding. At DIV12, OPC were added to the axonal chamber to achieve myelination, as described previously. At DIV24, the chambers were imaged on a Leica SP8 microscope with temperature control at 37° C. and 5% CO₂ flow with a 25x water immersion objective (Leica). Per chamber all mEOS2 positive mitochondria in axons within 2 fields of view in the axonal chamber and the adjacent microchannels were then converted using the 405 nm laser at 3% laserpower for 2 minutes. To assess ARMD chambers were demyelinated using 0.005 mg/ml lysolecithin for 2 hours. The chambers were then returned to the incubator overnight. The following day photoconverted regions were imaged to assess newly transported mitochondria (green) from the cell body chamber to the axonal chamber. SCoRe was used to determine the myelinated status of the axons. To analyse the amount of newly transported green mitochondria in the axonal chamber, 20 axons were randomly selected, cut out and saved as single axon images. Fiji was then used to calculate the proportion of green in red area. To calculate the axonal mitochondrial occupancy, the “analyse particles” function of Fiji was used to determine the mitochondrial area, which was then corrected for the length of the axonal segment. Each dot in FIG. 11, represents an axon.

### Assessing the Effect of Mitochondrial Manipulations on Lysosomal Trafficking in Unmyelinated DRG Neurons

To determine the effect of Miro1, PGC1α and pioglitazone on lysosomal trafficking in DRG neurons, unmyelinated DRG neurons were seeded on glass-bottomed 35 mm dishes, as previously described. Miro1 was added at seeding, while PGC1α and pioglitazone were added to the DRG neurons at 10 days and 6 days before imaging, respectively. At DIV14 LysoTracker (LysoTracker Red DND-99, L7528 Invitrogen) was added to the culture medium at a concentration of 50 nM and incubated for 30 minutes at 37° C., before washing the LysoTracker off and adding live imaging solution to the DRG neurons. Live fluorescence imaging was performed as described for microfluidic chambers, using a 63x oil immersion objective (Plan-Apochromat 1.40 NA Oil DIC M27 objective, Zeiss, Germany). Videos and Kymographs were generated as described previously. The total number of lysosomes moving in both directions were counted on the kymograph and visually confirmed on the videos and the direction of movement for every single lysosome was noted. Each datapoint on FIG. 2 (F&G) represents the average number of moving lysosomes per axon.

### Pharmacological Inhibition of Complex IV in DRG Neurons Using Sodium Azide

Sodium azide (Sigma S8032, UK) was used to inhibit complex IV in unmyelinated DRG neurons, cultured on glass bottom dishes, as described previously [39]. Firstly, a concentration gradient experiment was performed to determine the sublethal sodium azide (Sigma S8032, UK) dose for DRG neurons, which inhibits complex IV, by trypan blue exclusion test [67]. The highest dose, which did not impact cell viability and resulted in complex IV inhibition, was determined as 100 µM sodium azide for 17 hours, which was used for all the subsequent complex IV inhibition experiments. Complex IV histochemistry was performed as described below and images were obtained using bright field microscopy. The intensity of the complex IV reactive product was assessed using FIJI and densitometry to analyse complex IV activity at a single cell level.

For live imaging of mitochondrial dynamics in complex IV deficient DRG neurons, unmyelinated DRG neurons were cultured on glass bottom dishes, as described previously. Lentivirus mEOS2 was added at seeding and live imaging was performed at DIV21, as described previously. Kymograph were prepared and mitochondria were analysis as described previously for Purkinje cell axons in cerebellar slices.

### Complex IV Inhibition and Seahorse Analysis of Mitochondrial Respiration

DRG neuronal cells were cultured on V7 Seahorse 24-well cell culture microplates (Agilent Technologies), in maintenance medium in a 5% CO₂ 37° C. incubator. Sodium azide was present in media at either 0.1 or 1 mM for 17 hours before the Seahorse experiment, and the sodium azide was maintained in the media during the Seahorse run. Plates were incubated for 30 mins at 37° C. (without CO₂), before entry into the Seahorse XFe24 Extracellular Flux Analyser (Agilent). Three measurements were taken basally, and three measurements taken after injection of each drug to either inhibit ATP-linked respiration, uncouple respiration or inhibit the respiratory chain. Mitochondrial respiration was calculated by subtracting the first OCR measurement following injection of antimycin/rotenone from the third basal OCR measurement. Normalisation of OCR to relative protein content was achieved following Sulforhodamine B (SRB) staining of all cell wells. Data for each treatment groups was averaged from between 4-5 replicate wells.

### Human Tissue

Frozen human autopsy tissue was obtained, including dorsal root ganglia and spinal cord blocks, from the rapid autopsy program at Cleveland Clinic, Ohio, USA and Netherland Brain Bank (Table 1). The frozen tissue blocks were stored at -80° C. until cryosectioning. The entire DRG were cryosectioned at 15 micrometers intervals. Cryosections were then subjected to COX/SDH histochemistry, COX/immunofluorescent labeling as well as laser micro dissection of single neurons, as described below.

### Complex IV/Complex II Histochemistry and Analysis of Complex IV Deficient DRG Neurons

Mitochondrial respiratory chain complex IV(COX)/complex ll (succinate dehydrogenase [SDH]) activity was assessed using the well-established sequential COX/SDH histochemistry, as previously described[9]. In five randomly chosen DRG cryosections, complex IV deficient neurons (stained blue) were calculated as a percentage of total neurons (sum of neurons stained either brown or blue) in both frozen human (Table 1) and frozen mouse tissue (Table 2 and COX10Adv mutant mice). For the detection of complex IV deficient cells with intact complex ll within the brain spinal cord of the animal models (Table 2), the inventors stained every 5^{th} section of the entire brain and spinal cord and scanned the sections at x40 objective manually to look for cells stained blue (complex IV deficient with intact complex II).

### Sequential COX/Immunofluorescent Labeling of DRG Neurons and CNS Cells

To identify complex IV in proprioceptive and nociceptive DRG neurons, NF200 and peripherin were immunofluorescently labelled following completion of complex IV histochemistry step, as previously described [38]. Both brightfield and fluorescent images of five randomly chosen DRG cryosections were obtained using Zeiss ApoTome.2 microscope (Zeiss, Germany) and superimposed using ImageJ to identify complex IV activity within proprioceptive and nociceptive DRG neurons. Proprioceptive neurons were identified as NF200+peripherin-. To identify complex IV deficient CNS cells in animal models, independent of complex ll activity, the inventors performed sequential COX/immunofluorescent histochemistry and used antibodies against COX-l and complex ll 70 kDa (Table 3), as previously described [38]. To look for immunofluorescently labelled cells, the inventors stained every 5^{th} section of the entire brain and spinal cord, except marmoset EAE, where tissue blocks were used, and manually scanned the sections at x40 objective.

### Laser Capture of Single DRG Neurons From MS Tissue and Grey Matter From Animal Models for Real Time PCR and Long Range PCR

Cryosections (15 µm thick) were mounted onto membrane slides (Leica) for laser micro dissection. Following COX/SDH histochemistry, single DRG neurons were micro dissected using a Leica laser micro dissection microscope (Leica LMD), as previously described[9]. Spinal cord grey matter regions from snap frozen tissue was micro dissected from animal models (Table 2), except in cuprizone model where approximately 250 × 250 µm² region of cortex was included. DNA extraction was carried out using the QIAamp DNA Micro Kit (Qiagen).

Real-time PCR, as previously described [9], was used to analyse the level of mitochondrial DNA deletion in single DRG neurons in MS tissue. Known deletion-level standards, a blood-positive control and a blood-negative control, run in triplicate, were added to the assays. Long-range PCR, as previously described [9], was used to detect mtDNA deletions in human autopsy tissue and in snap frozen tissue from animal models.

### Cresyl Violet Staining of DRG Neurons

One in every 5 serial sections of each DRG was processed using cresyl violet staining and the total number of DRG neurons with nuclei was counted per DRG section for each animal and human case. An average of 4-5 DRG per animal and human case was included in each data point shown in FIGS. 13a-c, and FIG. 17a).

### Animal Genotyping and Focal Lesioning of the Spinal Cord Dorsal Columns

To generate inducible knockout of complex IV subunit 10 (COX10) in DRG neurons, the inventors crossed COX10^{flox/flox} mice with Advillin^{CreERT2/+} mice to derive COX10^{flox/flox} Advillin^{CreERT2/+} mutant mice (COX10Adv mutants) [13, 48]. Wild type and COX10Adv mutant mice were maintained on C57Bl6 background. Mice were genotyped, as previously described [13, 48]. Tamoxifen was dissolved in sunflower oil/ at 20 mg/ml and gavaged at a dose of 60 ul/10 g body weight daily over 5 consecutive days. On an Applied Biosystems 7500 Fast Real Time PCR system in triplicates, qPCR was performed using 10 ng of genomic DNA in a 12.5-µl assay using PowerSYBR Green PCR Master Mix (Applied Biosystems). A 167-base-pair Cox10^{flox}-specific fragment was obtained with primers 5′-CGGGGATCAATTCGAGCTCGCC-3′ and 5′-CACTGACGCAGCGCCAGCATCTT-3′. All animal experiments were performed in compliance with Animals (scientific procedures) Act 1986 and UK Home Office guidelines under the animal license (PPL 70/7872). Tissue from established animal models was obtained through collaborations as listed in Table 2.

Both wild type mice and COX10Adv mutant mice of C57Bl6 background, aged approximately 13 weeks, were anaesthetized using inhalation of 3-4% isoflurane/oxygen with supplementation of 0.05 ml of buprenorphine administered subcutaneously. Following exposure of spinal vertebrae at T12/T13, a dorsal laminectomy was performed to expose the dura and the central vein. Dura just lateral to the central vein was pierced using a sterile dental needle. The tip of a pulled glass capillary, attached to a Hamilton syringe, was introduced into the dorsal column through the pieced dura at an angle of 45 degree, approximately, and 0.05 µl of 1% lysolecithin was injected to cause focal demyelination of the dorsal column in mice. Wild type mice were euthanized at 3, 5, 7 and 9 days post lesioning for the time course experiments (FIG. 1) and both wild type mice and COX10Adv mice were euthanized at 3 days post lesioning for the axon protection experiments (FIG. 3 and FIG. 6).

### Animal Behaviour

Both wild type mice and COX10Adv mutant mice of C57Bl6 background were subjected to no more than two behavioral tests, following a period of training. Animal in the survival experiment that reached a moderate severity as defined in the project license were culled using a Schedule 1 method.

### Electron Microscopy of Mouse Spinal Cord

Mice were euthanized using an overdose of pentobarbitone (200 mg/ml) and perfused intravascularly with 2.5% glutaraldehyde and 4% paraformaldehyde in 0.1 M sodium cacodylate buffer. Following post fixing of the spinal cord, tissue was prepared, embedded in Durcupan resin and stained, as previously described [63]. A JEOL JEM-1400 Plus transmission electron microscope was used with Gatan one view digital camera and Digital Micrograph 3 software at x5700 magnification to image dorsal column axons in cross section. To calculate the g ratio, the cross-sectional area of the axon, both including and excluding myelin ring, was determined using the freehand tool on ImageJ software, which enable the radius of the axon and radius of the axon plus myelin ring to be calculated to determine the g-ratio. At least 100 dorsal column axons in the gracile fasciculus in the thoracic spinal cord were included for each mouse.

### Synaptoneurosome Preparation and Measurement of Ca²⁺ Fluorescence Responses

To investigate whether COX10 deficiency or focal dorsal column demyelination impact on neurotransmission at the first central synapses of DRG neurons, synaptoneurosomes from dorsal column nuclei (DCN) [24, 73] were prepared. Protocols that optimise metabolic and ionic integrity in synaptoneurosomes have been recently developed so dynamic Ca²⁺ fluorescence responses to receptor stimuli can be measured [43, 68, 74]. The selective AMPA receptor agonist, R,S-AMPA (Abcam) with the selective inhibitor of AMPA receptor desensitization, cyclothiazide (Tocris) or the Ca²⁺ ionophore, ionomycin (Abeam), were added immediately before recording. Intracellular Ca²⁺ fluorescence was read at excitation 488 nm, emission 518 nm. Ionomycin (10 µM) and basal measurements were included in every plate to calibrate the dynamic range of the assay. Mean responses were calculated over the first 4 min following drug addition.

### Sequential COX Histochemistry and Triple Label Immunofluorescence Histochemistry

Axonal complex IV activity and complex IV subunit-I: COX histochemistry was combined with triple immunofluorescent histochemistry, using with antibodies against neurofilament, complex IV subunit-I and complex II 70 kDa. This staining was done to assess: 1) complex IV activity in single axons, and, 2) identify complex IV-deficient axonal mitochondria and, subsequently, assess the complex subunit status of mitochondria that lack complex IV activity. This sequential technique, which is performed in the same tissue section and has been described and validated in previous publications, is based on the observation that only the mitochondria lacking complex IV activity are immunofluorescently labeled following COX histochemistry, because the brown deposit of COX histochemical reaction in complex IV active mitochondria prevents the immunolabeling of the mitochondrial respiratory chain complex subunits. COX media consisted of 100 µM cytochrome c, 4 mM diaminobenzidine tetrahydrochloride and 20 µg/ml catalase in 0.1 M phosphate buffer pH7.0. Cryosections were incubated at 37° C. for 30 min and washed in PBS. Cryosections were then proceeded through the triple label immunofluorescent histochemistry steps, as stated above. Secondary antibodies were as follows: anti-mouse IgG2a 488, Life Technologies; anti-mouse IgG1 633, Life Technologies; anti-chicken 488 respectively). The sequentially stained sections were mounted in glycerol with Hoechst nuclear stain and stored at -20° C. until required for imaging by the Zeiss Imager Z1 Apotome 2 microscope.

### Microscopy

Sequential COX histochemistry and immunofluorescent histochemistry: brightfield images of complex IV activity and immunofluorescent labeling of axons as well as mitochondria that lack complex IV activity, due to the blocking of immunolabeling by the deposits of complex IV histochemical reaction, were obtained using the Apotome microscope. SCoRE was used to ensure confirmation of myelinated and demyelinated axons in these sections.

In adjacent sections complex IV activity and immunofluorescent labeling of APP and synaptophysin were similarly captured. Images were taken using a x63 oil lens and brightfield and FITC, TRIC and Cy5 channels were imaged sequentially.

Brightfield microscopy: 1 in 5 serial cryosections of the entire brain and/or entire length of the spinal cord, stained for COX/SDH, were viewed on an Olympus BX51 microscope at x20 magnification to screen for respiratory-deficient cells with intact complex II activity (stained blue) by two investigators independently.

### Statistical Analysis

GraphPad Prism® software, version 6.0 (GraphPad Software Inc, US) was used for statistical analysis. For normally distributed data, a two-way student’s t-test was used to determine statistical significance, while One-way ANOVA was used for multiple column comparisons. For data that was not normally distributed the two-tailed ‘Mann-Whitney-U’ test was selected as a non-parametric test, while multiple column comparisons were done by using the Kruskal-Wallis test. Unless otherwise specified, data is represented as the average ± standard deviation (SD). A p-value < 0.05 was regarded as being significant and designated *⁻**** in the graphs.

### Results

### Axonal Mitochondrial Content Increases Following Demyelination Through Mobilization of Mitochondria From Neuronal Cell Body to Axon, Forming an Axonal Response of Mitochondria to Demyelination (ARMD)

The inventors considered that mitochondria within healthy neuronal cell bodies respond to demyelination by moving to the axon. Therefore, the inventors labelled mitochondria with a photoconvertible dye (mito-mEOS2) and performed live imaging over 20 minutes to visualise mitochondria that enter the myelinated and demyelinated axons from the cell body in cerebellar slices (FIG. 1). Following photoconversion of axonal mitochondria, the inventors identified a large increase in the number of unconverted mitochondria moving from the Purkinje cell body to the proximal axon segment upon demyelination (FIGS. 1c, e-gand videos 6-10, online resource), compared to control (FIGS. 1b, d, f-g). Furthermore, the motile mitochondria displayed a greater anterograde speed in demyelinated axons compared with myelinated axons (FIG. 1h). These effects of demyelination on axonal mitochondria are not an artefact of the demyelinating agent, lysolecithin, since it did not significantly impact mitochondrial movement in Shiverer mice where axons lack myelin (FIG. 9). To assess this mitochondrial response from cell body to the demyelinated axon in another neuronal subtype, the inventors cultured DRG neurons in the cell body compartment of microfluidic chambers, myelinated their axons in a separate chamber, induced demyelination by exposing axonal compartment to lysolecithin and again found evidence of increased mitochondrial mobilisation from the neuronal cell body to the axon and increased axonal mitochondrial content (FIG. 10). This homeostatic response, whereby axonal mitochondrial content increases following demyelination through the mobilization of mitochondria from the neuronal cell body to axon, the axonal response of mitochondria to demyelination has been termed ARMD by the inventors.

In order to study the effects of demyelination on mitochondria in DRG neuronal cell body versus axon, which projects to the central nervous system and peripheral nervous system, the inventors cultured myelinated DRG neurons in microfluidic chambers (FIG. 9). Similarly, the inventors found evidence of ARMD in this experimental system.

### Homeostatic ARMD Is Not Sufficient to Protect the Acutely Demyelinated Axons From Degeneration

Because the increased transport of mitochondria from the neuronal cell body to the axon upon demyelination requires time to build up axon mitochondrial content in the demyelinated axon, the inventors quantitated changes in axonal mitochondrial content over time. ARMD peaked at 5 days post demyelination in cerebellar slices and at 7 days post demyelination of centrally protecting dorsal column axons of DRG neurons, in vivo. To examine the impact of ARMD on axonal health, the inventors compared the temporal changes of axonal degeneration, indicated by the formation of axonal bulbs [70], with that of axonal mitochondrial content (FIGS. 1i-l). In both demyelinated cerebellar slices and the centrally protecting dorsal column axons of DRG neurons in vivo, transection of Purkinje cell and DRG axons occurred 2-4 days prior to the peak of ARMD (FIGS. 1i-l). It was concluded that the acutely demyelinated axon is particularly vulnerable for at least 2-4 days, until ARMD reaches its peak, signifying a potential therapeutic window for neuroprotection.

### Targeting Mitochondrial Dynamics and Biogenesis Increases the Mobilisation of Mitochondria From Neuronal Cell Body to the Axon

Given that the movement of mitochondria from the neuronal cell body to increase the mitochondrial content of the acutely demyelinated axon is a relatively protracted process compared with the rapid degeneration of the axon, the inventors aimed to determine whether the influx of mitochondria from the cell body to the axon can be increased by over-expression of Mitochondrial Rho GTPase1 (Miro1), which is known to facilitate mitochondrial transport by tethering mitochondria to a motor/adaptor protein complex [22]. This was found to significantly increase the movement of mitochondria from the cell body to the axon in unmyelinated DRG neurons (FIGS. 2a-b), as expected [61]. However, targeting mitochondrial transport alone did not significantly affect the total mitochondrial content of the axons, due to the large number of stationary mitochondria present there (FIG. 2biv-v). Therefore, the inventors stimulated mitochondrial biogenesis in neurons by over-expressing the peroxisome proliferator-activated receptor gamma (PPAR-γ) coactivator 1-alpha (PGC1α), which is the master regulator of mitochondrial biogenesis (FIG. 2c) [44, 75]. Over-expression of PGC1α in DRG neurons led to increased anterograde mitochondrial transport and increased mitochondrial axonal content, thus mimicking ARMD. The result of the genetic manipulation was recapitulated upon pharmacological application of pioglitazone, an established PGC1α pathway agonist (FIG. 2d) [44]. These findings were specific to mitochondria, since lysosomal transport was unaffected (FIG. 2e). The inventors also examined mitochondrial content in myelinated axons following over-expression of Miro1, PGC1α and application of pioglitazone, and found similar changes to unmyelinated axons (FIGS. 2h-j). Taken together, these findings indicate the potential of targeting mitochondrial dynamics and biogenesis in neurons to enhance ARMD.

### Promoting ARMD in Healthy Neurons Protects the Acutely Demyelinated Axons From Degeneration

In order to assess whether enhancing the mobilisation of mitochondria from the neuronal cell body to the axon alone or in combination with increased biogenesis could protect the acutely demyelinated axons from degeneration, the inventors considered three experimental systems. First, they found that the over-expression of Miro1, PGC1α, or the application of pioglitazone specifically to the DRG neuronal cell bodies in microfluidic chambers, significantly decreased the fragmentation of acutely demyelinated axons and significantly increased the number of intact demyelinated axons (FIGS. 3a-c). Addition of a PGC1α inhibitor to the neuronal cell body chamber together with pioglitazone reversed the protective effect of pioglitazone treatment on demyelinated axons, implicating PGC1α pathway in pioglitazone induced neuroprotection (FIG. 3c). They also applied pioglitazone to cerebellar slice cultures and noted a significant increase in the axonal mitochondrial content and a significant decrease in axonal degeneration following demyelination (FIGS. 3d-f). Further, they administered pioglitazone to wild type mice for 6 weeks prior to focal demyelination of the dorsal columns and found a significantly decreased axonal bulb formation with treatment (FIGS. 3j-l). Given the pleiotropic effects of pioglitazone, the inventors investigated whether expression of its target, PGC1α, was affected in DRG neurons and found a significant increase of PGC1α+ nuclei in DRG neurons with treatment (FIG. 11). In keeping with in vitro findings in DRG neurons following over expression of PGC1α and pioglitazone treatment, the inventors found a significant increase in mitochondria content within axons, in vivo, with pioglitazone treatment (FIG. 3m). Taken together, these in vitro and in vivo findings indicate that the increased mobilisation of mitochondria from the neuronal cell body to the axon by targeting of PGC1α pathway and over-expression of Miro1 enhances ARMD in wild type neurons and protects acutely demyelinated axons from degeneration.

As pioglitazone protection of acutely demyelinated axons could in principle be due to improved myelin debris clearance and peroxisomal function in neurons, impaired microglial activation or targeting of PGC1a in neurons, PGC1a and mitochondria were assessed within DRG neurons in wild type mice that received dietary pioglitazone.

### Dorsal Root Ganglia Neurons in Progressive Multiple Sclerosis Display Complex IV Deficiency and an Increase in Mitochondrial Content in Demyelinated Axons

In disease states, such as progressive MS, there is perturbation to the function of mitochondria in neurons, exemplified by complex IV deficiency, the terminal complex of the electron transport chain. Complex IV deficiency is known to impair anterograde mitochondrial transport in myelinated axons and deplete the mitochondrial content of myelinated axons [6, 9, 14, 32, 33, 53, 78]. This raises the question whether mitochondria in these complex IV deficient neurons in disease states can respond to demyelination. Therefore, the inventors considered the relevance of ARMD to demyelinating diseases with complex IV deficient neurons, by examining respiratory deficient DRG neurons in MS autopsy tissue and the mitochondrial parameters of their demyelinated axons at the dorsal root entry zone [6, 9, 14, 78].

The inventors studied DRG neuronal cell bodies and their demyelinated centrally projecting axons in the spinal cord dorsal columns in progressive MS, as this enabled the accurate identifications of mitochondria in cell bodies and associated demyelinated axons. The inventors found approximately one third of neuronal cell bodies in DRG of MS to be complex IV deficient (FIGS. 4a-c) due to clonally expanded mitochondrial DNA deletions (FIG. 12). Histological analysis of DRG in MS revealed a significant increase in the number of HLA+ and GFAP+ cells while the neuronal cell body count did not differ significantly between MS and controls, indicating a reactive milieu (FIG. 13,). In 6 out of 18 progressive MS cases, the inventors identified chronically demyelinated axons in dorsal columns at the dorsal root entry zone (FIG. 4d), and found positive correlations between the percentage of complex IV deficient proprioceptive neuronal cell bodies in the DRG and the mitochondrial content, mitochondrial area, mitochondrial number and impaired complex IV in associated demyelinated dorsal column axons (FIG. 4e). This statistical association indicates that complex IV deficient neuronal cell bodies, harboring clonally expanded mtDNA deletion, respond to demyelination by mobilising mitochondria to the axon, despite their respiratory deficiency.

TABLE 1

Details of human autopsy cases *Cleveland Clinic. Netherland Brain Bank and Edinburgh Brain Bank

| Case No | Age/Gender | Subtype | Disease duration | Cause of death | PMD | C and L | |
|---|---|---|---|---|---|---|---|
| MS1 | 34/M | MS | | | <24 | C3 | L3 |
| MS2 | 41/F | SPMS* | 11 | End stage MS (natural death) | 8 | C6 | L2 |
| MS3 | 56/M | SPMS* | 14 | GI bleed | 10 | C6 | |
| MS4 | 64/M | PPMS | 34 | End stage MS (natural death) | 8 | | L1 |
| MS5 | 69/F | SPMS | 26 | Viral infection | 13 | C5 | L3 |
| MS6 | 77/F | Progressive MS | >6 yrs | Pneumonia | 7 | | L2 |
| MS7 | 40/F | Progressive relapsing MS | 8 | Haematemesis | 9 | | L5 |
| MS8 | 50/F | SPMS | 17 | Euthanasia | 7 | C8 | L1 |
| MS9 | 53/M | SPMS | 9 | Cardiopulmonary failure | 5 | | L5 |
| MS 10 | 61/M | SPMS* | 31 | Euthanasia | 9 | | L5 |
| MS 11 | 71/F | Progressive MS | 23 | Respiratory failure | 10 | | L2 |
| MS 12 | 78/F | SPMS | 25 | Cerebrovascular accident | 11 | C7 | L3 |
| MS 13 | 81/F | SPMS | 59 | Pneumonia | 7 | | L1 |
| MS 14 | 82/M | SPMS | 32 | Pneumonia | 6 | C8 | |
| MS 15 | 88/F | PPMS | 25 | Chronic colitis | 8 | | L5 |
| MS 16* | 62/F | SPMS | 43 | Pneumonia | 6 | | L |
| MS 17 | 70/M | SPMS | 46 | Cardiac arrest | 7 | | L2 |
| MS 18* | 74/M | SPMS | 36 | Respiratory failure | 9 | | L |
| CON 1 | 74/M | - | - | Lung carcinoma | 7 | | L2 |
| CON 2 | 58/M | - | - | Oligodendroglioma (left parieto-occipital) | 5 | C | L2 |
| CON 3 | 71/F | - | - | Renal failure | 7 | C | L |
| CON 4 | 68/F | - | - | Sepsis | 42 | | L |
| CON 5 | 70/M | - | - | Cerebrovascular accident | 24 | | L |
| CON 6 | 55/F | - | - | Sepsis | 24 | | L |
| CON 7 | 62/F | - | - | Euthanasia (renal cell carcinoma) | 8 | | L2 |
| CON 8 | 80/M | - | - | Cardiac failure | 8 | C | L1 |
| CON 9 | 92/F | - | - | Urosepsis | 7 | | L1 |
| CON 10 | 84/M | - | - | Cardiopulmonary failure | 5 | | L2 |
| CON 11 | 71/M | - | - | Pneumonia | 7 | C | |
| CON 12 | 47/F | - | - | Breast carcinoma | 4 | C | |
| PD 1 | 84/M | - | - | Pneumonia | 7 | | L |
| PD 2 | 84/F | - | - | Old age | 6 | | L |
| PD 3 | 79/F | - | - | Renal failure | 5 | | L |
| MND 1 | 58/M | - | - | Respiratory failure | 6 | | L |
| MND 2 | 74/M | - | - | Pneumonia | 7 | | L |

### Existing Animal Models of MS Lack Complex IV Deficient Neurons and Complex IV Knockout Mice Exhibit the ARMD

To investigate ARMD in complex IV deficient neurons, the inventors studied 9 distinct experimental models of demyelination that are pertinent to MS and a model of traumatic axonal transection (Table 2). However, they did not find complex IV deficient neurons within the brain, spinal cord or DRG in any of these models (FIG. 13) and mitochondrial DNA deletions were rarely detected in any of these animal models (FIG. 15). Therefore, to model the complex IV deficient DRG neurons that are observed in MS, the inventors developed a neuron-specific inducible mitochondrial mutant by knocking out complex IV subunit 10 (COX10 or protohaem IX farnesyltransferase) in DRG neurons (COX10Adv mutant mice, FIG. 5) [13, 31]. In 13 week old COX10Adv mutants, 59% of proprioceptive DRG neurons were complex IV deficient (FIG. 5c), but showed no signs of behavioural disruption or neurodegeneration (FIGS. 16 + 17). Similar to the observations in MS autopsy tissue, the inventors found that mitochondrial content, area and complex IV deficiency was significantly increased in demyelinated axons in COX10Adv mutant mice (FIG. 5l-o). Mitochondrial content of myelinated axons did not significantly differ in COX10Adv mutant mice compared with controls (FIG. 18). The fact that the ARMD occurs in complex IV deficient neurons in an experimental model provided the inventors with the opportunity to test whether enhancing ARMD can protect these demyelinated axons, which are acutely vulnerable to degeneration.

### Promoting ARMD in Complex IV Deficient Neurons Is Neuroprotective

Given that complex IV deficient neurons demonstrate ARMD, the inventors tested whether transport of mitochondria from complex IV deficient neuronal cell bodies to the axon can be enhanced by targeting mitochondrial over-expression of Miro1, PGC1α and pioglitazone treatment. The inventors inhibited complex IV and therefore mitochondrial respiration in mature DRG neurons, in vitro, by using sodium azide (SA), which resulted in a significantly decreased anterograde transport of mitochondria, as expected (FIGS. 6a-c) [39]. Strikingly, over-expression of Miro1 or PGC1α and treatment with pioglitazone overcame the anterograde mitochondrial transport deficit present in complex IV deficient neurons (FIGS. 6e-f). Furthermore, targeting mitochondrial biogenesis with PGC1α over-expression and pioglitazone treatment limited the SA induced inhibition of mitochondrial respiration, presumably due to the increased mitochondrial content in neurons (FIG. 6d and FIG. 19) [44]. Finally, the inventors tested whether enhancing the ARMD in complex IV deficient neurons may also be neuroprotective, in vivo, in the context of demyelination. Consistent with the inventor’s in vitro findings, the treatment of COX10Adv mutant mice with dietary pioglitazone for 6 weeks significantly increased axonal mitochondrial content and the percentage of proprioceptive neurons with PGC1α-positive nuclei (FIG. 20). Dietary pioglitazone neither change the number of nuclei, identified by DAPI staining, nor the number of Iba1 positive microglia in focal lesions, although there was a trend towards decreasing Iba1 positive microglia with treatment (FIG. 21) [46, 66]. Strikingly, the increased mobilisation of mitochondria in complex IV deficient neurons and axonal mitochondrial content, by pioglitazone treatment, protected the acutely demyelinated axons as evident by the significant decrease in acutely transected axons in demyelinated lesions (FIGS. 6g-i).

To determine whether the protection of acutely demyelinated axons in COX10Adv mutant mice affects functional connectivity, the inventors evaluated the excitability of the first central synapses of the dorsal column axons in the dorsal column nuclei (DCN). The inventors isolated functionally intact synapses from DCN, using synaptoneurosomal preparations, and exposed to AMPA receptor agonists to assess Ca²⁺ responses. AMPA-induced Ca²⁺ fluorescence responses of freshly prepared DCN synaptoneurosomes were significantly reduced in COX10Adv mutant mice compared to wild type controls and the deficit was exacerbated 3 days following dorsal column demyelination (FIG. 6j). Importantly, pioglitazone treatment significantly improved the excitability of DCN synaptoneurosome derived from the complex IV deficient neurons that were experimentally demyelinated (FIG. 6j). Thus, pioglitazone treatment protects not only the structural integrity of acutely demyelinated axons in COX10Adv mutant mice, but also downstream synaptic function.

TABLE 2

Details of established experimental disease models

| Model (co-author) | Species (strain) | Time points for analysis in days | n= (brain, spinal cord, DRG) |
|---|---|---|---|
| Focal demyelinating dorsal funiculus lesion: | Mouse | 5* | 0, 6, 6 |
| LPC (1%) [79] | (C57BL/6) | | |
| LPS (200 ng) [16] | | | |
| | Rat (Sprague-Dawley) | 7* | 0, 6, 3 |
| Cuprizone-mediated demyelination of the brain[21] | Mouse | 42* | 6, 0, 0 |
| | (C57BL/6) | 91 | 6, 0, 0 |
| TMEV-induced inflammatory demyelination [57] | Mouse | 7 (acute encephalitis) | 3, 3, 3 |
| | (SJL/J) | 41 (demyelinating) | 3, 3, 3 |
| | | 112 (axonal loss)* | 3, 3, 3 |
| | | 209 (chronic) | 3, 3, 3 |
| T-reg depleted active EAE with MOG35-55 [42] | Mouse | 13 (acute)* | 0, 10, 0 |
| | (C57BL/6) | 30 (resolution) | 0, 7, 0 |
| | | 60 (chronic) | 0, 9, 0 |
| Humanized TCR transgenic with spontaneous EAE [15] | Mouse | 120-150*, (clinical | 3, 3, 0 |
| | (C57BL/6) | score 1-2 and >3) | 3, 3, 0 |
| Chronic EAE with subcutaneous spinal cord homogenate[2] | Mouse | 18 (acute)* | 3, 3, 3 |
| | (Biozzi ABH) | 35-40 (relapsing) | 3, 3, 3 |
| | | 120 (chronic) | 10, 10, 6 |
| Acute EAE with rMOG [12] | Rat | 1 | 3, 3, 0 |
| | (Dark Agouti, Harlan) | 3 | 3, 3, 0 |
| | | 14* | 3, 3, 3 |
| EAE with rMOG34-56 [26] | Marmoset | 11 days*, on average, post EAE score of 2.5 | 9, 5, 2 |
| | (Callithrix jacchus) | | |

*indicates peak clinical disease or peak demyelination time point for the analysis of axonal mitochondrial parameters. All the time points stated above were included in the detection for respiratory-deficient cells. DRG: dorsal root ganglia. EAE: experimental autoimmune encephalomyelitis. LPC: lysolecithin. LPS: lipopolysaccharide. MOG: myelin oligodendrocyte glycoprotein. TCR: T-cell receptor. TMEV: Theiler’s murine encephalomyelitis virus. n= number of animals used for brain and spinal cord analysis. Equal numbers of age-matched controls were used, except for marmoset EAE where 4 age-matched controls were used.

The inventors have shown demyelination creates a state of energy deficiency in the axon, through insufficient energy producing capacity, despite a homeostatic mechanism, termed ARMD, whereby mitochondria move from the cell body to the axon and gradually increase the mitochondrial content of the demyelinated axon. The protection of acutely demyelinated axons by enhancing ARMD further support the existence of an energy deficient state in acutely demyelinated axons. The inventors consider myelination during development and the resulting energy efficiency of nerve impulse conduction decreases the requirement for mitochondria in the axon (brake on) and demyelination undo the energy efficient state and increase the need for mitochondria in disease states (brake off), necessitating ARMD. During the timeframe until the homeostatic ARMD reaches its optimal level, the acutely demyelinated axon is particularly vulnerable to degeneration. A brake on mitochondria by myelin is elegantly illustrated by the healthy optic nerve, where mitochondria are sparsely distributed in the myelinated axonal segments compared with the abundance of mitochondria within the unmyelinated segments in laminar cribrosa. Furthermore, myelinated axons in wild type mice contain less mitochondria than those with mutation of myelin genes, where myelination is incomplete.

The inventors consider the neuroprotective strategies discussed herein can be utilised to preserve the acutely demyelinated axons, and then neuroregenerative therapies to remyelinate these axons can be provided. Remyelination addresses the long term saving of axons (chronically demyelinated) that have survived the acute myelin attack. The inventors strategy to enhance ARMD is about making more axons survive the attack on myelin, so that they can subsequently be remyelinated. For remyelination to save the acutely demyelinated axons it needs to occur rapidly, before the homeostatic ARMD is completed (within the first few days of demyelination). Even if extremely efficient remyelination is achieved such axons are likely to undergo repeated demyelination in the hostile microenvironment. Furthermore, remyelination is an imperfect regenerative process, in terms of axonal mitochondria. The inventors have found that the mitochondrial content of remyelinated axons remained elevated relative to myelinated axons. Nevertheless, remyelination therapy can be used to regulate the axonal mitochondrial content and avoid potential long-term adverse effects of increasing axonal mitochondrial content following enhanced ARMD.

In summary, the inventors have determined a compensatory role for mitochondria as part of the neuronal response to demyelination. Although the mobilisation of mitochondria from the neuronal cell body to the axon occurs spontaneously following the destruction of myelin, the resultant increase in the mitochondrial content of the demyelinated axon, which the inventors have termed homeostatic ARMD, is too protracted. Enhancing ARMD, by increasing the transport of mitochondria from the neuronal cell body to the axon as well as mitochondrial biogenesis in the neuron, protects the acutely demyelinated axon. This novel neuroprotective strategy is considered to be applicable to all demyelinating disorders, even when neurons are respiratory chain deficient. Hence, drugs that enhance ARMD are important to protect the vulnerable acutely demyelinated axons, so that regenerative strategies, like remyelination, can be effectively implemented in demyelinating CNS and PNS disorders.

TABLE 3

Details of antibodies used for immunohistochemistry and immunofluorescent histochemistry

| | | | |
|---|---|---|---|
| Antigen | Target | Antibody type | Source |
| NF-L | Neurofilament light | Chicken polyclonal | EnCor |
| NF200 | Neurofilament | Rabbit polyclonal | Sigma |
| | heavy | | |
| SMI31 | Phosphorylated neurofilaments | Mouse IgG₁ | Covance |
| SMI32 | Non-phosphorylated neurofilaments | Mouse IgG₁ | Covance |
| MBP | Myelin basic protein | Rat polyclonal | Covance |
| MBP | Myelin basic protein | Rabbit polyclonal | Gift from Peter Brophy |
| COX-I | COX subunit-I | Mouse IgG₂ₐ | Abcam |
| COX-IV | COX subunit-IV | Mouse IgG₂ₐ | Abcam |
| SDHA II | SDH 70 kDa subunit | Mouse IgG₁ | Abcam |
| Complex I 20 kDa subunit | NDUFS7 protein | Mouse IgG₁ | Abcam |
| Complex I 30 kDa subunit | NDUFS3 protein | Mouse IgG₁ | Abcam |
| Porin | Mitochondrial transmembrane protein | Mouse IgG_{2b} | Abcam |
| PGC1a | peroxisome proliferator-activated receptor gamma coactivator 1-alpha | Rabbit polyclonal | Abcam |
| Peripherin | Peripherin | Mouse IgG₁ | Sigma |
| Iba1 | Ionised calcium binding adaptor molecule 1 | Rabbit polyclonal | MenaPath |

Although the invention has been particularly shown and described with reference to particular examples, it will be understood by those skilled in the art that various changes in the form and details may be made therein without departing from the scope of the present invention.

### Increased Mitochondrial Content Within Demyelinated Axons Indicates ARMD in Experimental Disease Models Irrespective of the Mode of Demyelination

The inventors assessed the mitochondrial content of demyelinated axons in nine different disease models at peak clinical disease or peak demyelination time point, when a clinical phenotype is not apparent (Table 4) This was then compared with myelinated axons in controls (Table 5). Mitochondria within demyelinated axons are evident in confocal images based on immunofluorescent labeling of porin, which is a voltage gated anion channel (VDAC) expressed in all mitochondria, neurofilament and myelin basic protein (MBP) (FIG. 21). The mitochondrial content within demyelinated axons was significantly increased in the spinal cord of all EAE, LPC, LPS and TMEV induced models as well as in the corpus callosum in cuprizone mediated demyelination, compared with myelinated axons in controls, indicating ARMD (FIG. 21 and Table 4). The significant increase in mitochondrial content within demyelinated axons arose from increased mitochondrial size and/or increased mitochondrial number (Table 5). In LPC, cuprizone mediated demyelination and TMEV induced demyelination the average axonal mitochondrial size was significantly greater than in myelinated axons. Meanwhile, the greater mitochondrial number accounted for the increased mitochondrial content in EAE models. These observations show that ARMD is a consistent phenomenon irrespective of the mode of demyelination.

TABLE 4

Details of disease models

| Model (Reference) | species (strain) | time points for analysis in days | n= (brain, spinal cord) |
|---|---|---|---|
| focal demyelinating dorsal funiculus lesion: LPC (1%) (79) | Mouse (C57BL/6) | 5* | 0, 6 |
| LPS (200 ng) (16) | Rat (Sprague-Dawley) | 7* | 0, 6 |
| Cuprizone-mediated demyelination of the brain (21) | Mouse (C57BL/6) | 42* | 6, 0 |
| TMEV-induced inflammatory demyelination (57) | Mouse (SJL/J) | 41 (demyelinating) | 3, 3 |
| T-reg depleted active EAE with MOG₃₅₋₅₅ (42) | Mouse (C57BL/6) | 13 (acute)* | 0,10 |
| humanized TCR transgenic with spontaneous EAE (15) | Mouse (C57BL/6) | 120-150*, (clinical score 1-2 and >3) | 3, 3 |
| chronic EAE with subcutaneous spinal cord homogenate (2) | Mouse (Biozzi ABH) | 18 (acute)* | 3, 3 |
| acute EAE with rMOG (12) | Rat (Dark Agouti, Harlan) | 14* | 3, 3 |
| EAE with rMOG₃₄₋₅₆ (26) | Marmoset (Callithrix jacchus) | 11 days*, on average, post EAE score of 2.5 | 9, 5 |

*indicates peak clinical disease or peak demyelination time point for the analysis of axonal mitochondrial parameters. All the time points stated above were included in the detection for respiratory-deficient cells. EAE: experimental autoimmune encephalomyelitis. LPC: lysolecithin. LPS: lipopolysaccharide. MOG: myelin oligodendrocyte glycoprotein. TCR: T-cell receptor. TMEV: Theiler’s murine encephalomyelitis virus. n= number of animals used for brain and spinal cord analysis. Equal numbers of age-matched controls were used, except for marmoset EAE where 4 age-matched naive controls were used.

TABLE 5

Changes in axonal mitochondrial content, size and number following demyelination in models and MS

| model | mitochondrial content (% of axon area) | mitochondrial size (µM²) | mitochondrial number/10³ µm² of axon area |
|---|---|---|---|
| focal LPC | 28.78 ± 9.98*** | 5.28 ± 1.77** | 42.07 ± 19.39 |
| | 15.02 ± 3.57 | 2.93 ± 1.56 | 3.28 ± 19.36 |
| focal LPS | 23.88 ± 12.98*** | 4.55 ± 2.24 | 56.40 ± 25.57* |
| | 13.17 ± 3.73 | 4.58 ± 1.30 | 40.11 ± 14.02 |
| cuprizone-mediated | 23.01 ± 4.99*** | 4.75 ± 1.71** | 68.81 ± 12.21*** |
| | 15.02 ± 3.57 | 2.92 ± 1.03 | 47.66 ± 7.71 |
| TMEV-induced | 20.39 ± 2.88** | 6.61 ± 2.29** | 34.71 ± 8.62 |
| | 15.17 ± 2.04 | 4.56 ± 1.29 | 33.45 ± 10.92 |
| TCR tg EAE | 16.76 ± 7.30* | 5.01 ± 1.09 | 40.76 ± 20.51* |
| | 10.15 + 4.00 | 4.26 ± 1.21 | 23.30 ± 13.13 |
| EAE Biozzi ABH | 20.81 ± 7.42*** | 4.93 ± 1.29 | 43.04 ± 14.74** |
| | 12.79 ± 3.22 | 4.69 ± 1.24 | 27.75 ± 5.16 |
| T-reg depleted EAE | 23.18 ± 9.79*** | 2.57 ± 0.97 | 53.46 ± 14.77 |
| | 15.38 ± 4.88 | 1.98 ± 1.42 | 32.04 ± 16.96 |
| EAE rat | 25.74 ± 7.72*** | 5.10 ± 2.62 | 54.90 ± 13.78*** |
| | 13.37 ± 3.91 | 3.78 + 1.24 | 37.15 ± 10.39 |
| EAE marmoset | 19.51 ± 5.92* | 3.08 ± 0.64 | 64.93 ± 21.44 |
| | 14.92 ± 8.33 | 3.31 ± 1.93 | 53.18 ± 12.49 |
| Progressive MS (81) | 19.61 ± 5.67** | 15.67 ±3.68** | 49.21 ± 11.93 |
| | 6.26 ± 1.73 | 5.22 ± 1.92 | 33.60 ± 9.67 |

Mitochondrial content (column two) is expressed as a percentage of axonal area occupied by porin labelled elements. Mitochondrial size (column three) and mitochondrial number (column four) are based on the area and number, respectively, of porin labelled elements within axons in confocal images. Shaded rows indicate mean values for myelinated axons from controls and unshaded rows mean indicate values for demyelinated axons. EAE: experimental autoimmune encephalomyelitis. LPC: lysolecithin. LPS: lipopolysaccharide. TCR tg: T-cell receptor transgenic. TMEV: Theiler’s murine encephalomyelitis virus. Values indicate mean ± standard deviation. *p<0.05, **p<0.01 and ***p<0.001.

### Increase in Axonal Mitochondrial Content Is Not Always Accompanied by a Corresponding Increase in Mitochondrial Respiratory Chain Complex IV Activity

To determine whether the increased mitochondrial content within demyelinated axons is reflected at the functional level, the inventors assessed complex IV activity of mitochondria at a single axon level using sequential COX histochemistry and immunofluorescent labelling of axons in snap frozen serial cryosections (FIG. 22). This sequential technique labels mitochondria with complex IV activity (in brighfield image). Furthermore, this technique identifies mitochondria that lack complex IV activity (in immunofluorescent images) and enables us to determine the subunit status of mitochondria that lack complex IV activity. Quantitation of complex IV activity within axons revealed a significantly greater area of the demyelinated axons occupied by complex IV active mitochondria in LPC lesions (FIG. 22 and Table 6). In these LPC-induced focal lesions, complex IV active mitochondria with elongated morphology are prevalent within demyelinated axons (FIG. 22). The remaining models did not show a significant increase in complex IV activity within demyelinated axons. Although complex IV activity had a tendency to increase within demyelinated axons in TMEV and cuprizone models the difference was not statistically significant.

TABLE 6

Complex IV activity and complex IV subunit-l relative to axonal area and complex IV subunit-I relative complex II subunit labelled area in myelinated axons and demyelinated axons

| model | complex IV activity in all axonal mitochondria: % of axon area (Reference) | complex IV subunit-I in complex IV-deficient mitochondria: % of complex II 70 KDa area | complex IV subunit-I in all axonal mitochondria, % of axonal area |
|---|---|---|---|
| Focal LPC | 12.81 ± 3.69** | 85.49 ± 16.96 | 24.15 ± 8.37** |
| | 7.44 ± 4.57 | 62.22 ± 11.59 | 10.25 ± 2.43 |
| Focal LPS | 6.08 ± 2.55 | 63.16 ± 21.29 | 15.51 ± 8.43** |
| | 5.84 ± 1.78 | 69.13 ± 14.51 | 8.49 ± 2.48 |
| Cuprizone-mediated | 10.50 ± 6.55 | 68.73 ± 14.24 | 17.30 ± 3.75** |
| | 6.83 ± 1.55 | 61.34 ± 11.98 | 11.08 ±2.25 |
| TMEV-induced | 9.42 ± 6.37 | 58.41 ± 14.14 | 15.89 ± 2.91** |
| | 4.98 ± 1.19 | 62.88 ± 10.87 | 10.89 ± 2.44 |
| EAE, TCR tg | 4.28 ± 2.48 | 73.46 ± 15.32 | 11.61 ± 5.31* |
| | 4.59 ± 1.38 | 67.25 ± 11.14 | 7.72 ± 2.87 |
| EAE, Biozzi ABH | 3.69 ± 2.15 | 62.36 ± 21.20 | 15.16 ± 5.28** |
| | 5.07 ± 2.16 | 67.54 ± 10.26 | 8.76 ± 2.09 |
| EAE, T-reg depleted | 2.18 ± 1.15 | 64.73 ± 18.56 | 14.24 ± 4.96* |
| | 6.10 ± 1.11 | 71.96 ± 11.90 | 9.32 ± 2.23 |
| EAE, rat | 4.36 ± 1.90 | 64.78 ± 20.50 | 17.59 ± 5.28** |
| | 5.57 ± 2.50 | 67.83 ± 12.17 | 8.12 ± 2.38 |
| EAE, marmoset | 5.01 ± 1.39 | 66.49 ± 22.95 | 14.71 ± 4.46* |
| | 5.74 ± 1.98 | 70.76 ±18.49 | 10.13 ± 5.66 |
| Progressive MS | 7.37 ± 4.98** (39) | 22.82 ± 21.34** | 10.10 ± 3.89** |
| | 2.23 ± 1.62 | 51.89 ± 22.86 | 4.91 ± 3.05 |

Mitochondrial respiratory chain complex IV active mitochondria in axons (column two) are assessed as a percentage of axonal area occupied by these complex IV active mitochondria. Complex IV subunit-l is assessed in all axonal mitochondria as the percentage area of the subunit present within the axons (column three) in triple labeled images. When axonal mitochondria that lack complex IV activity are detected (using complex II 70 kDa labeled elements within axons by the sequential COX histochemistry and triple labeling technique), the percentage area of complex IV subunit-I labeling in the mitochondria are not significantly different between myelinated axons in controls and demyelinated axons in all the disease models (last column). Shaded rows indicate values for myelinated axons and unshaded rows indicate values for demyelinated axons. EAE: experimental autoimmune encephalomyelitis. LPC: lysolecithin. LPS: lipopolysaccharide. TCR tg: T-cell receptor transgenic. TMEV: Theiler’s murine encephalomyelitis virus. Values indicate mean ± standard deviation. *p<0.01 and **p<0.001.

### Mitochondrial Respiratory Chain Complex IV subunit-I Is Preserved in Complex IV Deficient Axonal Mitochondria in All Experimental Disease Models

In axonal mitochondria that lack complex IV activity, the extent of complex IV subunit-I (COX-I) labelling was similar in demyelinated axons compared with myelinated axons, suggesting that that the lack of complex IV activity is not caused by the loss of complex IV subunit-I. To confirm that the complex IV subunit is intact in demyelinated axons, the inventors immunofluorescently co-labelled mitochondria and the subunit in serial sections and found a significant increase in complex IV subunit-I within demyelinated axons compared with myelinated axons (Table 6). The regions with inflammatory infiltrates in EAE showed a diffuse loss of complex IV activity and upregulation of iNOS. These findings suggest that complex IV activity is acutely inhibited and/or complex IV subunits are post-translationally modified by the inflammatory response in a proportion of axonal mitochondria that had responded to demyelination.

### Mitochondrial Complex IV Activity in Demyelinated Axons Inversely Correlates With Axonal Injury in Experimental Disease Models

The inventors assessed the relationship between axonal mitochondrial parameters (content and complex IV activity) and axonal damage as indicated by the density of APP and synaptophysin positive elements (FIG. 23). The inventors did not detect a significant correlation between axonal mitochondrial content and axonal damage. However, at the level of complex IV activity there is a significant inverse correlation between the mean complex IV activity within demyelinated axons and the density of synaptophysin positive elements (FIG. 23). APP positive elements showed a similar inverse relationship with axonal complex IV activity, although not statistically significant. The inverse correlation between axonal complex IV activity and axonal damage indicates the importance of preserving complex IV activity in acutely demyelinated axons.

In summary, the inventors have found:
Increased mitochondrial content within demyelinated axons indicates ARMD in experimental disease models irrespective of the mode of demyelination.
Increased mitochondrial content within demyelinated sciatic nerve axons indicates ARMD is a phenomenon which can occur in the peripheral nervous system (FIG. 25).
The number of sciatic axonal bulbs was enhanced in both WT and mutant mouse models, when mice were treated with pioglitazone (FIG. 26).
Pioglitazone treatment significantly increased axonal mitochondrial content in non-demyelinated axons and tended to increase axonal mitochondrial content in demyelinated axons (demyelinated using lysolecithin) (FIG. 27).
increase in axonal mitochondrial content is not always accompanied with a corresponding increase in mitochondrial respiratory chain complex IV activity.
The reduction in mitochondrial respiratory chain complex IV activity is not due to a reduction in mitochondrial respiratory chain complex IV subunit-I chain expression.
Mitochondrial complex IV activity in demyelinated axons inversely correlates with axonal injury in experimental disease models.
Enhancing ARMD as a neuroprotective strategies may be further optimised by limiting damage to complex IV through the combination with immunomodulatory therapy.

### REFERENCES

1. Absinta M, Sati P, Masuzzo F, Nair G, Sethi V, Kolb H et al (2019) Association of Chronic Active Multiple Sclerosis Lesions With Disability In Vivo. JAMA Neurol 76:1474-1483. doi: 10.1001/jamaneurol.2019.2399
2. AI-Izki S, Pryce G, O′Neill JK, Butter C, Giovannoni G, Amor S et al (2012) Practical guide to the induction of relapsing progressive experimental autoimmune encephalomyelitis in the Biozzi ABH mouse. Mult Scler Relat Disord 1:29-38. doi: 10.1016/j.msard.2011.09.001
3. Andrews H, White K, Thomson C, Edgar J, Bates D, Griffiths I et al (2006) Increased axonal mitochondrial activity as an adaptation to myelin deficiency in the Shiverer mouse. J Neurosci Res 83:1533-1539. doi: 10.1002/jnr.20842
4. Birgbauer E, Rao TS, Webb M (2004) Lysolecithin induces demyelination in vitro in a cerebellar slice culture system. J Neurosci Res 78:157-166. doi: 10.1002/jnr.20248
5. Bristow EA, Griffiths PG, Andrews RM, Johnson MA, Turnbull DM (2002) The distribution of mitochondrial activity in relation to optic nerve structure. Arch Ophthalmol Chic III 1960 120:791-796. doi: 10.1001/archopht.120.6.791
6. Broadwater L, Pandit A, Clements R, Azzam S, Vadnal J, Sulak M et al (2011) Analysis of the mitochondrial proteome in multiple sclerosis cortex. Biochim Biophys Acta 1812:630-641. doi: 10.1016/j.bbadis.2011.01.012
7. Campbell GR, Kraytsberg Y, Krishnan KJ, Ohno N, Ziabreva I, Reeve A et al (2012) Clonally expanded mitochondrial DNA deletions within the choroid plexus in multiple sclerosis. Acta Neuropathol (Berl) 124:209-220. doi: 10.1007/s00401-012-1001-9
8. Campbell GR, Reeve AK, Ziabreva I, Reynolds R, Turnbull DM, Mahad DJ (2013) No excess of mitochondrial DNA deletions within muscle in progressive multiple sclerosis. Mult Scler 19:1858-1866. doi: 10.1177/1352458513490547
9. Campbell GR, Ziabreva I, Reeve AK, Krishnan KJ, Reynolds R, Howell O et al (2011) Mitochondrial DNA deletions and neurodegeneration in multiple sclerosis. Ann Neurol 69:481-492. doi: 10.1002/ana.22109
10. Chernoff GF (1981) Shiverer: an autosomal recessive mutant mouse with myelin deficiency. J Hered 72:128. doi: 10.1093/oxfordjournals.jhered.a109442
11. Dal-Bianco A, Grabner G, Kronnerwetter C, Weber M, Höftberger R, Berger T et al (2017) Slow expansion of multiple sclerosis iron rim lesions: pathology and 7 T magnetic resonance imaging. Acta Neuropathol (Berl) 133:25-42. doi: 10.1007/s00401-016-1636-z
12. Davies AL, Desai RA, Bloomfield PS, Mclntosh PR, Chapple KJ, Linington C et al (2013) Neurological deficits caused by tissue hypoxia in neuroinflammatory disease. Ann Neurol 74:815-825. doi: 10.1002/ana.24006
13. Diaz F, Thomas CK, Garcia S, Hernandez D, Moraes CT (2005) Mice lacking COX10 in skeletal muscle recapitulate the phenotype of progressive mitochondrial myopathies associated with cytochrome c oxidase deficiency. Hum Mol Genet 14:2737-2748. doi: 10.1093/hmg/ddi307
14. Dutta R, McDonough J, Yin X, Peterson J, Chang A, Torres T et al (2006) Mitochondrial dysfunction as a cause of axonal degeneration in multiple sclerosis patients. Ann Neurol 59:478-489. doi: 10.1002/ana.20736
15. Ellmerich S, Takacs K, Mycko M, Waldner H, Wahid F, Boyton RJ et al (2004) Disease-related epitope spread in a humanized T cell receptor transgenic model of multiple sclerosis. Eur J Immunol 34:1839-1848. doi: 10.1002/eji.200324044
16. Felts PA, Woolston A-M, Fernando HB, Asquith S, Gregson NA, Mizzi OJ et al (2005) Inflammation and primary demyelination induced by the intraspinal injection of lipopolysaccharide. Brain J Neurol 128:1649-1666. doi: 10.1093/brain/awh516 17. Ferguson B, Matyszak MK, Esiri MM, Perry VH (1997) Axonal damage in acute multiple sclerosis lesions. Brain J Neurol 120 ( Pt 3):393-399. doi: 10.1093/brain/120.3.393
18. Franklin RJM, ffrench-Constant C, Edgar JM, Smith KJ (2012) Neuroprotection and repair in multiple sclerosis. Nat Rev Neurol 8:624-634. doi: 10.1038/nrneurol.2012.200
19. Frischer JM, Weigand SD, Guo Y, Kale N, Parisi JE, Pirko I et al (2015) Clinical and pathological insights into the dynamic nature of the white matter multiple sclerosis plaque. Ann Neurol 78:710-721. doi: 10.1002/ana.24497
20. Fünfschilling U, Supplie LM, Mahad D, Boretius S, Saab AS, Edgar J et al (2012) Glycolytic oligodendrocytes maintain myelin and long-term axonal integrity. Nature 485:517-521. doi: 10.1038/nature11007
21. Goldberg J, Clarner T, Beyer C, Kipp M (2015) Anatomical Distribution of Cuprizone-Induced Lesions in C57BL6 Mice. J Mol Neurosci MN 57:166-175. doi: 10.1007/s12031-015-0595-5
22. Guo X, Macleod GT, Wellington A, Hu F, Panchumarthi S, Schoenfield M et al (2005) The GTPase dMiro is required for axonal transport of mitochondria to Drosophila synapses. Neuron 47:379-393. doi: 10.1016/j.neuron.2005.06.027
23. Haider L, Fischer MT, Frischer JM, Bauer J, Höftberger R, Botond G et al (2011) Oxidative damage in multiple sclerosis lesions. Brain J Neurol 134:1914-1924. doi: 10.1093/brain/awr128
24. Hollingsworth EB, McNeal ET, Burton JL, Williams RJ, Daly JW, Creveling CR (1985) Biochemical characterization of a filtered synaptoneurosome preparation from guinea pig cerebral cortex: cyclic adenosine 3′:5′-monophosphate-generating systems, receptors, and enzymes. J Neurosci Off J Soc Neurosci. doi: 5:2240-2253
25. Irrcher I, Adhihetty PJ, Sheehan T, Joseph A-M, Hood DA (2003) PPARgamma coactivator-1alpha expression during thyroid hormone- and contractile activity-induced mitochondrial adaptations. Am J Physiol Cell Physiol 284:C1669-1677. doi: 10.1152/ajpcell.00409.2002
26. Jagessar SA, Kap YS, Heijmans N, van Driel N, van Straalen L, Bajramovic JJ et al (2010) Induction of progressive demyelinating autoimmune encephalomyelitis in common marmoset monkeys using MOG34-56 peptide in incomplete freund adjuvant. J Neuropathol Exp Neurol 69:372-385. doi: 10.1097/NEN.0b013e3181d5d053
27. Kapoor R, Furby J, Hayton T, Smith KJ, Altmann DR, Brenner R et al (2010) Lamotrigine for neuroprotection in secondary progressive multiple sclerosis: a randomised, double-blind, placebo-controlled, parallel-group trial. Lancet Neurol 9:681-688. doi: 10.1016/S1474-4422(10)70131-9
28. Kiryu-Seo S, Ohno N, Kidd GJ, Komuro H, Trapp BD (2010) Demyelination increases axonal stationary mitochondrial size and the speed of axonal mitochondrial transport. J Neurosci Off J Soc Neurosci 30:6658-6666. doi: 10.1523/JNEUROSCI.5265-09.2010
29. Krishnan KJ, Reeve AK, Samuels DC, Chinnery PF, Blackwood JK, Taylor RW et al (2008) What causes mitochondrial DNA deletions in human cells? Nat Genet 40:275-279. doi: 10.1038/ng.f.94
30. Lassmann H (2018) Multiple Sclerosis Pathology. Cold Spring Harb Perspect Med 8:. doi: 10.1101/cshperspect.a028936
31. Lau J, Minett MS, Zhao J, Dennehy U, Wang F, Wood JN et al (2011) Temporal control of gene deletion in sensory ganglia using a tamoxifen-inducible Advillin-Cre-ERT2 recombinase mouse. Mol Pain 7:100. doi: 10.1186/1744-8069-7-100
32. Lax NZ, Whittaker RG, Hepplewhite PD, Reeve AK, Blakely EL, Jaros E et al (2012) Sensory neuronopathy in patients harbouring recessive polymerase γ mutations. Brain J Neurol 135:62-71. doi: 10.1093/brain/awr326
33. Lehmann HC, Chen W, Borzan J, Mankowski JL, Höke A (2011) Mitochondrial dysfunction in distal axons contributes to human immunodeficiency virus sensory neuropathy. Ann Neurol 69:100-110. doi: 10.1002/ana.22150
34. Lewis TL, Kwon S-K, Lee A, Shaw R, Polleux F (2018) MFF-dependent mitochondrial fission regulates presynaptic release and axon branching by limiting axonal mitochondria size. Nat Commun 9:5008. doi: 10.1038/s41467-018-07416-2
35. Luchetti S, Fransen NL, van Eden CG, Ramaglia V, Mason M, Huitinga I (2018) Progressive multiple sclerosis patients show substantial lesion activity that correlates with clinical disease severity and sex: a retrospective autopsy cohort analysis. Acta Neuropathol (Berl) 135:511-528. doi: 10.1007/s00401-018-1818-y 36. MacKerron C, Robertson G, Zagnoni M, Bushell TJ (2017) A Microfluidic Platform for the Characterisation of CNS Active Compounds. Sci Rep 7:1-11. doi: 10.1038/s41598-017-15950-0
37. Mahad DJ, Trapp BD, Lassmann H (2015) Pathological mechanisms in progressive multiple sclerosis. Lancet Neurol 14:183-193. doi: 10.1016/S1474-4422(14)70256-X
38. Mahad DJ, Ziabreva I, Campbell G, Laulund F, Murphy JL, Reeve AK et al (2009) Detection of cytochrome c oxidase activity and mitochondrial proteins in single cells. J Neurosci Methods 184:310-319. doi: 10.1016/j.jneumeth.2009.08.020 39. Mahad DJ, Ziabreva I, Campbell G, Lax N, White K, Hanson PS et al (2009) Mitochondrial changes within axons in multiple sclerosis. Brain J Neurol 132:1161-1174. doi: 10.1093/brain/awp046
40. Mangeol P, Prevo B, Peterman EJG (2016) KymographClear and KymographDirect: two tools for the automated quantitative analysis of molecular and cellular dynamics using kymographs. Mol Biol Cell 27:1948-1957. doi: 10.1091/mbc.E15-06-0404
41. McCloskey C, Rada C, Bailey E, McCavera S, van den Berg HA, Atia J et al (2014) The inwardly rectifying K+ channel KIR7.1 controls uterine excitability throughout pregnancy. EMBO Mol Med 6:1161-1174. doi: 10.15252/emmm.201403944
42. McGeachy MJ, Stephens LA, Anderton SM (2005) Natural recovery and protection from autoimmune encephalomyelitis: contribution of CD4+CD25+ regulatory cells within the central nervous system. J Immunol Baltim Md 1950 175:3025-3032. doi: 10.4049/jimmunolX.175.5.3025
43. Mitchell R, Campbell G, Mikolajczak M, McGill K, Mahad D, Fleetwood-Walker SM (2019) A Targeted Mutation Disrupting Mitochondrial Complex IV Function in Primary Afferent Neurons Leads to Pain Hypersensitivity Through P2Y1 Receptor Activation. Mol Neurobiol 56:5917-5933. doi: 10.1007/s12035-018-1455-4
44. Morató L, Galino J, Ruiz M, Calingasan NY, Starkov AA, Dumont M et al (2013) Pioglitazone halts axonal degeneration in a mouse model of X-linked adrenoleukodystrophy. Brain 136:2432-2443. doi: 10.1093/brain/awt143
45. Mutsaers SE, Carroll WM (1998) Focal accumulation of intra-axonal mitochondria in demyelination of the cat optic nerve. Acta Neuropathol (Berl) 96:139-143. doi: 10.1007/s004010050873
46. Natrajan MS, Komori M, Kosa P, Johnson KR, Wu T, Franklin RJM et al (2015) Pioglitazone regulates myelin phagocytosis and multiple sclerosis monocytes. Ann Clin Transl Neurol 2:1071-1084. doi: 10.1002/acn3.260
47. Nave K-A (2010) Myelination and the trophic support of long axons. Nat Rev Neurosci 11:275-283. doi: 10.1038/nrn2797
48. Neumann E, Brandenburger T, Santana-Varela S, Deenen R, Köhrer K, Bauer I et al (2016) MicroRNA-1-associated effects of neuron-specific brain-derived neurotrophic factor gene deletion in dorsal root ganglia. Mol Cell Neurosci 75:36-43. doi: 10.1016/j.mcn.2016.06.003
49. Nikić I, Merkler D, Sorbara C, Brinkoetter M, Kreutzfeldt M, Bareyre FM et al (2011) A reversible form of axon damage in experimental autoimmune encephalomyelitis and multiple sclerosis. Nat Med 17:495-499. doi: 10.1038/nm.2324
50. O′Donnell KC, Vargas ME, Sagasti A (2013) WldS and PGC-1α Regulate Mitochondrial Transport and Oxidation State after Axonal Injury. J Neurosci 33:14778-14790. doi: 10.1523/JNEUROSCI.1331-13.2013
51. Ohno N, Chiang H, Mahad DJ, Kidd GJ, Liu L, Ransohoff RM et al (2014) Mitochondrial immobilization mediated by syntaphilin facilitates survival of demyelinated axons. Proc Natl Acad Sci U S A 111:9953-9958. doi: 10.1073/pnas.1401155111
52. Ohno N, Kidd GJ, Mahad D, Kiryu-Seo S, Avishai A, Komuro H et al (2011) Myelination and axonal electrical activity modulate the distribution and motility of mitochondria at CNS nodes of Ranvier. J Neurosci Off J Soc Neurosci 31:7249-7258. doi: 10.1523/JNEUROSCI.0095-11.2011
53. Powers JM, DeCiero DP, Cox C, Richfield EK, Ito M, Moser AB et al (2001) The dorsal root ganglia in adrenomyeloneuropathy: neuronal atrophy and abnormal mitochondria. J Neuropathol Exp Neurol 60:493-501. doi: 10.1093/jnen/60.5.493
54. Puigserver P, Wu Z, Park CW, Graves R, Wright M, Spiegelman BM (1998) A cold-inducible coactivator of nuclear receptors linked to adaptive thermogenesis. Cell 92:829-839. doi: 10.1016/s0092-8674(00)81410-5
55. Robertson G, Bushell TJ, Zagnoni M (2014) Chemically induced synaptic activity between mixed primary hippocampal co-cultures in a microfluidic system. Integr Biol Quant Biosci Nano Macro 6:636-644. doi: 10.1039/c3ib40221e
56. Samson AJ, Robertson G, Zagnoni M, Connolly CN (2016) Neuronal networks provide rapid neuroprotection against spreading toxicity. Sci Rep 6:1-11. doi: 10.1038/srep33746
57. Sathornsumetee S, McGavern DB, Ure DR, Rodriguez M (2000) Quantitative ultrastructural analysis of a single spinal cord demyelinated lesion predicts total lesion load, axonal loss, and neurological dysfunction in a murine model of multiple sclerosis. Am J Pathol 157:1365-1376. doi: 10.1016/S0002-9440(10)64650-0
58. Schain AJ, Hill RA, Grutzendler J (2014) Label-free in vivo imaging of myelinated axons in health and disease with spectral confocal reflectance microscopy. Nat Med 20:443-449. doi: 10.1038/nm.3495
59. Schindelin J, Arganda-Carreras I, Frise E, Kaynig V, Longair M, Pietzsch T et al (2012) Fiji: an open-source platform for biological-image analysis. Nat Methods 9:676-682. doi: 10.1038/nmeth.2019
60. Schuh C, Wimmer I, Hametner S, Haider L, Van Dam A-M, Liblau RS et al (2014) Oxidative tissue injury in multiple sclerosis is only partly reflected in experimental disease models. Acta Neuropathol (Berl) 128:247-266. doi: 10.1007/s00401-014-1263-5
61. Schwarz TL (2013) Mitochondrial trafficking in neurons. Cold Spring Harb Perspect Biol 5:. doi: 10.1101/cshperspect.a011304
62. Sharabi K, Lin H, Tavares CDJ, Dominy JE, Camporez JP, Perry RJ et al (2017) Selective Chemical Inhibition of PGC-1α Gluconeogenic Activity Ameliorates Type 2 Diabetes. Cell 169:148-160.e15. doi: 10.1016/j.cell.2017.03.001
63. Sherman DL, Krols M, Wu L-MN, Grove M, Nave K-A, GangloffY-G et al (2012) Arrest of myelination and reduced axon growth when Schwann cells lack mTOR. J Neurosci Off J Soc Neurosci 32:1817-1825. doi: 10.1523/JNEUROSCI.4814-11.2012
64. Sleigh JN, Weir GA, Schiavo G (2016) A simple, step-by-step dissection protocol for the rapid isolation of mouse dorsal root ganglia. BMC Res Notes 9:82. doi: 10.1186/s13104-016-1915-8
65. Sorbara CD, Wagner NE, Ladwig A, Nikić I, Merkler D, Kleele T et al (2014) Pervasive axonal transport deficits in multiple sclerosis models. Neuron 84:1183-1190. doi: 10.1016/j.neuron.2014.11.006
66. Storer PD, Xu J, Chavis J, Drew PD (2005) Peroxisome proliferator-activated receptor-gamma agonists inhibit the activation of microglia and astrocytes: implications for multiple sclerosis. J Neuroimmunol 161:113-122. doi: 10.1016/j.jneuroim.2004.12.015
67. Strober W (2001) Trypan blue exclusion test of cell viability. Curr Protoc Immunol Appendix 3:Appendix 3B. doi: 10.1002/0471142735.ima03bs21
68. Sun L, Gooding HL, Brunton PJ, Russell JA, Mitchell R, Fleetwood-Walker S (2013) Phospholipase D-mediated hypersensitivity at central synapses is associated with abnormal behaviours and pain sensitivity in rats exposed to prenatal stress. Int J Biochem Cell Biol 45:2706-2712. doi: 10.1016/j.biocel.2013.07.017
69. Toosy A, Ciccarelli O, Thompson A (2014) Symptomatic treatment and management of multiple sclerosis. Handb Clin Neurol 122:513-562. doi: 10.1016/B978-0-444-52001-2.00023-6
70. Trapp BD, Peterson J, Ransohoff RM, Rudick R, Mörk S, Bö L (1998) Axonal transection in the lesions of multiple sclerosis. N Engl J Med 338:278-285. doi: 10.1056/NEJM199801293380502
71. Trapp BD, Stys PK (2009) Virtual hypoxia and chronic necrosis of demyelinated axons in multiple sclerosis. Lancet Neurol 8:280-291. doi: 10.1016/S1474-4422(09)70043-2
72. Vaquié A, Sauvain A, Jacob C (2018) Modeling PNS and CNS Myelination Using Microfluidic Chambers. Methods Mol Biol Clifton NJ 1791:157-168. doi: 10.1007/978-1-4939-7862-5_12
73. Villasana LE, Klann E, Tejada-Simon MV (2006) Rapid isolation of synaptoneurosomes and postsynaptic densities from adult mouse hippocampus. J Neurosci Methods 158:30-36. doi: 10.1016/j.jneumeth.2006.05.008
74. Vinuela-Fernandez I, Sun L, Jerina H, Curtis J, Allchorne A, Gooding H et al (2014) The TRPM8 channel forms a complex with the 5-HT(1B) receptor and phospholipase D that amplifies its reversal of pain hypersensitivity. Neuropharmacology 79:136-151. doi: 10.1016/j.neuropharm.2013.11.006
75. Wareski P, Vaarmann A, Choubey V, Safiulina D, Liiv J, Kuum M et al (2009) PGC-1{alpha} and PGC-1{beta} regulate mitochondrial density in neurons. J Biol Chem 284:21379-21385. doi: 10.1074/jbc.M109.018911
76. Waxman SG (2006) Axonal conduction and injury in multiple sclerosis: the role of sodium channels. Nat Rev Neurosci 7:932-941. doi: 10.1038/nrn2023
77. Witte ME, Bø L, Rodenburg RJ, Belien JA, Musters R, Hazes T et al (2009) Enhanced number and activity of mitochondria in multiple sclerosis lesions. J Pathol 219:193-204. doi: 10.1002/path.2582
78. Witte ME, Nijland PG, Drexhage JAR, Gerritsen W, Geerts D, van Het Hof B et al (2013) Reduced expression of PGC-1α partly underlies mitochondrial changes and correlates with neuronal loss in multiple sclerosis cortex. Acta Neuropathol (Berl) 125:231-243. doi: 10.1007/s00401-012-1052-y
79. Woodruff RH, Franklin RJ (1999) Demyelination and remyelination of the caudal cerebellar peduncle of adult rats following stereotaxic injections of lysolecithin, ethidium bromide, and complement/anti-galactocerebroside: a comparative study. Glia 25:216-228. doi: 10.1002/(sici)1098-1136(19990201)25:3<216::aid-glia2>3.0.co;2-I
80. Zala D, Hinckelmann M-V, Yu H, Lyra da Cunha MM, Liot G, Cordelieres FP et al (2013) Vesicular glycolysis provides on-board energy for fast axonal transport. Cell 152:479-491. doi: 10.1016/j.cell.2012.12.029
81. Zambonin JL, Zhao C, Ohno N, Campbell GR, Engeham S, Ziabreva I et al (2011) Increased mitochondrial content in remyelinated axons: implications for multiple sclerosis. Brain J Neurol 134:1901-1913. doi: 10.1093/brain/awr110
82. Ziabreva I, Campbell G, Rist J, Zambonin J, Rorbach J, Wydro MM et al (2010) Injury and differentiation following inhibition of mitochondrial respiratory chain complex IV in rat oligodendrocytes. Glia 58:1827-1837. doi: 10.1002/glia.21052

1. A method of treating demyelinating disorders in a subject in need thereof, comprising the step of increasing mobilization of mitochondria from a neuronal cell body to a demyelinated axon, in vulnerable acutely demyelinated axons.

2. The method of claim 1 wherein the mobilisation is provided by increasing the synthesis or production of new mitochondria in neurons.

3. The method of claim 1 wherein the mobilisation is provided by the over expression of proteins that promote forward movement or anterograde transport of mitochondria, optionally the over expression of members of the kinesin family of proteins.

4. The method of claim 1 wherein the mobilisation is provided by over expression of peroxisome proliferator-activated receptor gamma (PPAR-γ) coactivator 1-alpha (PGC1a) or a down stream factor thereof which increase the mobilisation of mitochondria in neurons.

5. The method of claim 4 wherein a down stream factor is selected from a list comprising transcription factor A mitochondria (TFAM) and PPARS.

6. The method of claim 1 wherein mobilisation is induced by providing a thiazolidinedione which increases the mobilisation of mitochondria in neurons, optionally pioglitazone or rosiglitazone to a subject.

7. The method of claim 1, further comprising increasing axonal mitochondrial respiratory chain complex IV activity.

8. The method of claim 7, wherein the neuron(s) treated have deficient axonal mitochondrial respiratory chain complex IV activity.

9. The method of claim 7, wherein axonal mitochondrial respiratory chain complex IV activity is increased by limiting damage to the the axonal mitochondrial respiratory chain complex IV protein by using immunomodulatory therapy.

10. The method of claim 7, wherein axonal wherein axonal mitochondrial respiratory chain complex IV activity is increased by promoting mitochondrial biogenesis.

11. The method of claim 1 in combination with at least one step of
providing an immunomodulatory therapy,
providing a remyelination therapy, and
increasing the supply of metabolic substrate to the axon.

12. The method of claim 1 for treating a neuron in the central nervous system or peripheral nervous system.

13. A composition to increase mobilization of mitochondria from a neuronal cell body to a demyelinated axon in vulnerable acutely demyelinated axons, the composition selected from an agent that increases the synthesis of new mitochondria in neurons, an agent that promotes forward movement of mitochondria from a neuronal cell body to a demyelinated axon, or an agent that promotes anterograde transport of mitochondria from a neuronal cell body to a demyelinated axon for use in the treatment of a demyelinating disorder.

14. The composition for increasing mobilization of mitochondria from a neuronal cell body to a demyelinated axon in vulnerable acutely demyelinated axons for use in the treatment of a demyelinating disorder as claimed in claim 13, wherein the composition is a thiazolidinedione which increase the mobilisation of mitochondria in neurons.

15. The composition for increasing mobilization of mitochondria from a neuronal cell body to a demyelinated axon in vulnerable acutely demyelinated axons for use in the treatment of a demyelinating disorder as claimed in claim 13 wherein the composition is selected from pioglitazone and rosiglitazone.

16. A combination treatment comprising a composition for increasing mobilization of mitochondria from a neuronal cell body to a demyelinated axon in vulnerable acutely demyelinated axons and a remyelination agent.

17. A combination treatment comprising a composition comprising a modulator of the PGC1a/PPAR-γ pathway for increasing mobilization of mitochondria from a neuronal cell body to a demyelinated axon in vulnerable acutely demyelinated axons and a remyelination agent.

18. A combination treatment for use in the treatment of demyelinating disorders, comprising
a) a composition for increasing mobilization of mitochondria from a neuronal cell body to a demyelinated axon in vulnerable acutely demyelinated axons optionally wherein the composition is a thiazolidinedione which increase the mobilisation of mitochondria in neurons, optionally selected from pioglitazone and rosiglitazone, and
b) a remyelination agent.

19. A combination treatment for use in the treatment of MS, HIV neuropathy or diabetic neuropathy comprising
a) pioglitazone or rosiglitazone, and
b) a remyelination agent.

20. A combination treatment of claim 16 wherein the remyelination agent is selected from Metformin, Clemestine and Lipoic acid.

21. A combination treatment of claim 16 further comprising a composition which promotes the activity of axonal mitochondrial respiratory chain complex IV.

22. A composition comprising pioglitazone or rosiglitazone for use in the treatment of MS, HIV neuropathy or diabetic neuropathy.

23. An assay method to determine modulators of the PGC1a/PPAR-γ pathway comprising the steps
Providing an neuronal cell
Applying at least a first test agent to the neuronal cell
Determining whether the at least first test agent promotes increased mobilization of mitochondria from the neuronal cell body to the demyelinated axon
Wherein when there is increased mobilization of mitochondria from the neuronal cell body to the demyelinated axon it is indicative that the first test agent is a modulator of the PGC1a/PPAR-γ pathway.
